(19) United States
(12) Patent Application Publication
(10) Pub. No.: US 2025/0269086 A1
(43) Pub. Date: Aug. 28, 2025

(54) Cell Population for Transplantation and Method for Producing Same

(71) Applicants: RIKEN, Saitama (JP); Sumitomo Pharma Co., Ltd., Osaka (JP)

(72) Inventors: Michiko Mandai, Saitama (JP); Masayo Takahashi, Saitama (JP); Suguru Yamasaki, Kobe-shi (JP)

(73) Assignees: RIKEN, Saitama (JP); Sumitomo Pharma Co., Ltd., Osaka (JP)

(21) Appl. No.: 19/090,175

(22) Filed: Mar. 25, 2025

Related U.S. Application Data
(62) Division of application No. 16/463,424, filed on May 23, 2019, now Pat. No. 12,285,543.

(30) Foreign Application Priority Data
Nov. 25, 2016 (JP) 2016-229355
Jul. 14, 2017 (JP) 2017-138142

(51) Int. Cl. A61L 27/38; A61K 9/70; A61K 35/30; A61K 35/545; A61P 27/02; C12N 5/074; C12N 5/079; C12N 5/0793; C12N 5/10; C12N 15/85

(52) U.S. Cl. CPC A61L 27/3804; A61K 9/70; A61K 35/30; A61K 35/545; A61L 27/3839; A61P 27/02; C12N 5/0607; C12N 5/062; C12N 5/0621; C12N 5/0696; C12N 5/10; C12N 15/85

(57) ABSTRACT

An object of the present invention is to provide a cell population suitable for transplant of retinal tissue and a method of production thereof. The present invention provides a cell population for transplant, comprising retinal cells with a modified bipolar cell-regulating gene and a method of production thereof.

### SEQUENCE LISTING SUBMISSION VIA PATENT CENTER

The contents of the electronic sequence listing (sequencelisting.xml; Size: 72,633 bytes; and Date of Creation: Mar. 25, 2025) is herein incorporated by reference in its entirety.

### TECHNICAL FIELD

The present invention relates to a cell population for transplant and a method for producing the same.

### BACKGROUND ART

It has recently been reported that transplantation of photoreceptor progenitor cells at an appropriate differentiation stage into a normal retina in a living murine body results in the functional take of the cells (Non Patent Literature 1), and the possibility of transplantation therapy for degenerative diseases of photoreceptor cells such as retinitis pigmentosa has been shown.

Many methods for inducing differentiation of pluripotent stem cells to three-dimensional retinal tissues by self-organizing culture have been reported and it is becoming possible to produce and transplant three-dimensional retinal tissue having stratified structure. For example, methods have been reported for obtaining multilayered retinal tissue from pluripotent stem cells (Non-Patent Literature 2 and Patent Literature 1); for obtaining multilayered retinal tissue by forming an aggregate of homogeneous pluripotent stem cells in a serum-free culture medium containing a Wnt signaling pathway inhibitor, and culturing the obtained aggregate in suspension in the presence of a basement membrane preparation and then in a serum culture medium (Non Patent Literature 3, Patent Literature 2); and for obtaining retinal tissue by culturing an aggregate of pluripotent stem cells in suspension in a culture medium containing a BMP signaling pathway agent (Non-Patent Literature 4 and Patent Literature 3), and the like. Furthermore, it has been reported that transplanting retinal tissue obtained by induction of differentiation of pluripotent stem cells into a subject results in subsequent take, differentiation, and maturation of the transplant, while it has also been reported that the functional take of the transplant is not sufficient (Non Patent Literature 5).

Meanwhile, some genes involved in development or differentiation of retinal cells contained in retinal tissue in the living body have been reported.

For example, it has been reported that the ISL1 (Insulin gene enhancer protein 1, Islet-1) gene is expressed in the pancreas, the heart, the nerve, and the like and rod bipolar cells, cone bipolar cells, amacrine cells, and ganglion cells die in degeneration in the body of retina-specific ISL1 gene deletion mice (Non Patent Literature 6).

Moreover, it has been reported that the BHLHE23 (basic helix-loop-helix family, member e23) gene is expressed in the pancreas, the brain, the retina, and the like and rod bipolar cells die in degeneration in the body of BHLHE23 gene deletion mice (Non Patent Literature 7).

### CITATION LIST

### Patent Literature

Patent Literature 1:
International Publication No. WO 2011/055855
Patent Literature 2:
International Publication No. WO 2013/077425
Patent Literature 3:
International Publication No. WO 2015/025967

### Non Patent Literature

Non Patent Literature 1:
Nature, 444, 203-207 (2006)
Non Patent Literature 2:
Nature, 472, 51-56 (2011)
Non Patent Literature 3:
Cell Stem Cell, 10 (6), 771-785 (2012)
Non Patent Literature 4:
Nature Communications, 6, 6286 (2015)
Non Patent Literature 5:
Stem Cell Reports, 2, 662-674 (2014)
Non Patent Literature 6:
J Neurosci, 27 (46), 12, 12707-20 (2007)
Non Patent Literature 7:
Neuron, 43 (6), 779-93 (2004)

### SUMMARY OF INVENTION

### Technical Problem

An object of the present invention is to provide a cell population for transplant suitable for transplant of retinal tissue and a method for producing the same.

### Solution to Problem

The present inventors found that the functional take after transplantation of retinal tissue is improved by reducing bipolar cells in the retinal tissue of the transplant. More specifically, the present inventors made multiple examinations to achieve the aforementioned object and found that retinal cells having a genome in which a bipolar cell-regulating gene, described later, is modified do not sufficiently differentiate and mature into bipolar cells, but do differentiate and mature into photoreceptor cells without problems, in comparison with retinal cells having the wildtype genome, and that the ratio of contact between photoreceptor cells derived from the transplant and bipolar cells from the host is increased by use of the cell population for transplant (retinal tissue or the like) comprising the aforementioned retinal cells for transplant and the functional take after the transplant of the retinal tissue may be improved, thereby completing the present invention.

Accordingly, the present invention relates to:
[1] A cell population for transplant, comprising retinal cells having a modified bipolar cell-regulating gene.
[2] The cell population for transplant according to the aforementioned [1], wherein the cell population is in a form of cell suspension or cell aggregate.
[3] The cell population for transplant according to the aforementioned [1] or [2], wherein the bipolar cell-regulating gene is a gene encoding a transcriptional regulator.
[4] The cell population for transplant according to the aforementioned [3], wherein the bipolar cell-regulating gene is one or a plurality of genes selected from the group consisting of an ISL1 gene and a BHLHE23 gene.
[5] The cell population for transplant according to the aforementioned [4], wherein the ISL1 gene has a nucleotide sequence set forth in the following (1) or (2):
(1) a nucleotide sequence set forth in SEQ ID NO: 1, 4, or 7;
(2) a nucleotide sequence having a nucleotide sequence modified from the nucleotide sequence set forth in SEQ ID NO: 1, 4, or 7 by deletion, addition, insertion, or substitution of one or a plurality of nucleotides, and encoding a protein having 80% or more sequence identity with an amino acid sequence set forth in SEQ ID NO: 3, 6, or 9, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 3, 6, or 9.
[6] The cell population for transplant according to the aforementioned [4] or [5], wherein the BHLHE23 gene has a nucleotide sequence set forth in the following (1) or (2):
(1) a nucleotide sequence set forth in SEQ ID NO: 10 or 13;
(2) a nucleotide sequence having a nucleotide sequence modified from the nucleotide sequence set forth in SEQ ID NO: 10 or 13 by deletion, addition, insertion or substitution of one or a plurality of nucleotides, and encoding a protein having 80% or more sequence identity with an amino acid sequence set forth in SEQ ID NO: 12 or 15, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 12 or 15.
[7] The cell population for transplant according to any one of the aforementioned [1] to [6], wherein the modification of a bipolar cell-regulating gene comprises deletion of the gene.
[8] The cell population for transplant according to any one of the aforementioned [1] to [7], wherein the retinal cells are derived from a pluripotent stem cell.
[9] The cell population for transplant according to the aforementioned [8], wherein the pluripotent stem cell is an induced pluripotent stem cell or an embryonic stem cell.
[10] The cell population for transplant according to any one of the aforementioned [1] to [9], wherein the retinal cells comprise one or a plurality of cells selected from the group consisting of a retinal progenitor cell, a photoreceptor progenitor cell, and a photoreceptor cell.
[11] The cell population for transplant according to the aforementioned [10], wherein the retinal cells comprise one or a plurality of cells selected from a Chx10-positive cell, a Crx-positive cell, and a recoverin-positive cell.
[12] The cell population for transplant according to the aforementioned [10] or [11], wherein the sum of the number of retinal progenitor cells, photoreceptor progenitor cells, and photoreceptor cells is 10% or more of the total number of cells in the cell population for transplant.
[13] The cell population for transplant according to any one of the aforementioned [10] to [12], wherein the functional take rate after the transplant of the photoreceptor cells or photoreceptor cells induced from the retinal progenitor cell or photoreceptor progenitor cell after the transplant is improved.
[14] A culture of a cell population for transplant, comprising:
(1) a cell population for transplant according to any one of the aforementioned [1] to [13]; and
(2) a medium necessary to maintain viability of the cell population for transplant.
[15] A method for producing a cell population for transplant comprising retinal cells, comprising the following steps (1) and (2):
(1) modifying a bipolar cell-regulating gene of pluripotent stem cells to obtain in vitro, a cell population comprising the pluripotent stem cells with the modified bipolar cell-regulating gene;
(2) inducing differentiation of the cell population comprising the pluripotent stem cells obtained in step (1) into retinal cells in vitro to obtain a cell population for transplant comprising the retinal cells.
[16] The method of production according to the aforementioned [15], wherein the cell population for transplant comprising the retinal cells is in a form of cell suspension or cell aggregate.
[17] The method of production according to the aforementioned [15] or [16], wherein the bipolar cell-regulating gene is a gene encoding a transcriptional regulator.
[18] The method of production according to the aforementioned [17], wherein the bipolar cell-regulating gene is one or a plurality of genes selected from the group consisting of an ISL1 gene and a BHLHE23 gene.
[19] The method of production according to the aforementioned [18], wherein the ISL1 gene has a nucleotide sequence set forth in the following (1) or (2):
(1) a nucleotide sequence set forth in SEQ ID NO: 1, 4, or 7;
(2) a nucleotide sequence having a nucleotide sequence modified from the nucleotide sequence set forth in SEQ ID NO: 1, 4, or 7 by deletion, addition, insertion, or substitution of one or a plurality of nucleotides, and encoding a protein having 80% or more sequence identity with an amino acid sequence set forth in SEQ ID NO: 3, 6, or 9, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 3, 6, or 9.
[20] The method of production according to the aforementioned [18] or [19], wherein the BHLHE23 gene has a nucleotide sequence set forth in the following (1) or (2):
(1) a nucleotide sequence set forth in SEQ ID NO: 10 or 13;
(2) a nucleotide sequence having a nucleotide sequence modified from the nucleotide sequence set forth in SEQ ID NO: 10 or 13 by deletion, addition, insertion or substitution of one or a plurality of nucleotides, and encoding a protein having 80% or more sequence identity with an amino acid sequence set forth in SEQ ID NO: 12 or 15, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 12 or 15.
[21] The method of production according to any of the aforementioned [15] to [20], wherein the modification of a bipolar cell-regulating gene comprises deletion of the gene.
[22] The method of production according to any one of the aforementioned [15] to [21], wherein the pluripotent stem cells are induced pluripotent stem cells or embryonic stem cells.
[23] The method of production according to any one of the aforementioned [15] to [22], wherein the retinal cells comprise one or a plurality of cells selected from the group consisting of a retinal progenitor cell, a photoreceptor progenitor cell and a photoreceptor cell.
[24] The method of production according to the aforementioned [23], wherein the retinal cells comprise one or a plurality of cells selected from a Chx10-positive cell, a Crx-positive cell, and a recoverin-positive cell.
[25] The method of production according to the aforementioned [23] or [24], wherein the sum of the number of retinal progenitor cells, photoreceptor progenitor cells, and photoreceptor cells is 10% or more of the total number of cells in the cell population for transplant comprising the retinal cells.
[26] The method of production according to any one of the aforementioned [23] to [25], wherein the functional take rate after the transplant of the photoreceptor cells or photoreceptor cells induced from the retinal progenitor cell or photoreceptor progenitor cell after the transplant is improved.
[27] A method for treating a disease based on a disorder of retinal tissue or a condition of retinal tissue damage, comprising transplanting an effective amount of the cell population for transplant according to any one of the aforementioned [1] to [13] into a subject in need of transplant.
[28] A pharmaceutical composition for treating a disease based on a disorder of retinal tissue or a condition of retinal tissue damage, comprising the cell population for transplant according to any one of the aforementioned [1] to [13] as an active ingredient.
[29] The pharmaceutical composition according to the aforementioned [28], wherein the pharmaceutical composition is in a form of cell sheet. [30] A therapeutic agent for a disease based on a disorder of retinal tissue or a condition of retinal tissue damage, comprising the cell population for transplant according to any one of the aforementioned [1] to [13].
[31] The therapeutic agent according to the aforementioned [30], wherein the therapeutic agent is in a form of cell sheet.

### Advantageous Effects of Invention

According to the present invention, it becomes possible to provide a cell population for transplant suitable for transplant of retinal tissue and a method for producing the same.

### BRIEF DESCRIPTION OF DRAWINGS

FIG. 1 is a graph showing the ratio of rod bipolar cells, amacrine cells, and horizontal cells at a transplant site 40 to 50 days after transplant of the retinal tissue derived from mouse iPS cells wherein the function of a Bhlhb4 gene is deleted.

FIG. 2 is graphs showing the ratio of contact lengths of host rod bipolar cells and transplanted photoreceptor cells 40 to 50 days after or 90 to 100 days after transplant of the retinal tissue derived from mouse iPS cells wherein the function of a Bhlhb4 gene is deleted.

FIG. 3 is a graph showing the ratio of rod bipolar cells, amacrine cells, and horizontal cells at a transplant site 40 to 50 days after transplant of the retinal tissue derived from mouse iPS cells wherein the function of an ISL1 gene is deleted.

FIG. 4 is photographs showing synaptic connection between the host bipolar cells and the transplanted photoreceptor cells at a transplant site on day 30 to day 50 from transplant of the retinal tissue derived from mouse iPS cells wherein the function of a Bhlhb4 gene or an ISL1 gene is deleted.

FIG. 5 is confocal microscope photographs of the immunostained retinal tissue (tissue on day 29 after the initiation of inducing differentiation) derived from mouse iPS cells wherein the function of a Bhlhb4 gene or an ISL1 gene is deleted.

FIG. 6A is (upper row) a schematic diagram showing the design of CRISPR/Cas9 system for deleting the function of an ISL1 gene (SEQ ID NO: 3), (middle row) an agarose gel electrophoresis photograph for confirming the deletion of the ISL1 gene, and (lower row) a photograph of an established human ES cell line (ISL1 gene-deleted line).

FIG. 6B is drawings showing the sites of the ISL1 gene cleaved by the CRISPR/Cas9 system. Control sequence is SEQ ID NO: 22, No. 16 sequence is SEQ ID NO: 23, No. 19 sequence is SEQ ID NO: 24, gRNA sequence is SEQ ID NO: 20 and 21.

FIG. 7A is photographs showing that the human ES cell line wherein the function of the ISL1 gene is deleted is differentiated into a retina.

FIG. 7B is photographs showing the presence of photoreceptor progenitor cells in the retina derived from the human ES cell line wherein the function of the ISL1 gene is deleted.

FIG. 7C is photographs showing that the ISL1 protein is not expressed in the retina derived from the human ES cell line wherein the function of the ISL1 gene is deleted.

FIG. 8 is photographs showing that the retina derived from the human ES cell line wherein the function of the ISL1 gene is deleted has been taken after transplanted into a retinal degeneration rat.

FIG. 9A is photographs showing the ratio of bipolar cells and amacrine cells at a transplant site 240 to 260 days after transplant of the retina into which the human ES cell line wherein the function of the ISL1 gene is deleted is differentiated.

FIG. 9B is a graph showing the ratio of bipolar cells and amacrine cells at a transplant site 240 to 260 days after transplant of the retina into which the human ES cell line wherein the function of the ISL1 gene is deleted is differentiated.

### DESCRIPTION OF EMBODIMENTS

### 1. Regarding Cell Population for Transplant Comprising Retinal Cells

One embodiment of the present invention provides a cell population for transplant, comprising retinal cells with a modified bipolar cell-regulating gene. Retinal cells with a modified a bipolar cell-regulating gene do not sufficiently differentiate and mature into bipolar cells, but do differentiate and mature into photoreceptor cells without problems, in comparison with retinal cells having the wildtype genome. Therefore, the ratio of contact between photoreceptor cells derived from the transplant and bipolar cells from the host is increased by use of the cell population for transplant comprising the aforementioned retinal cells for transplant, and the functional take after the transplant of the retinal tissue may be improved. A detailed description will be provided below.

### 1-1. Cell Population for Transplant

The “cell population for transplant” in the present invention means a cell population prepared for transplant and used for transplant.

The “cell population” in the present invention means a population in which 2 or more of the same kind or different kinds of cells are present. Preferably, the cell population is present in a medium such as a culture medium. Cell populations include cell suspensions and cell aggregates and it is preferred that the cell population is in a form of cell suspension or cell aggregate.

The “cell suspension” in the present invention means a medium containing the same kind or different kinds of a plurality of cells in suspension. The suspension preferably refers to a state in which a majority (for example, 50% or more, 60% or more, 70% or more, 80% or more, 90% or more, or 95% or more) of cells present in a medium are dissociated from each other and exists without sustained physical contact. Some (for example, 50% or less, 40% or less, 30% or less, 20% or less, 10% or less or 5% or less) cells among the cells present in the medium may exist as cell aggregates or the like.

The “cell aggregate” in the present invention is not particularly limited, as long as it is a plurality of cells adhering to each other to form a mass and the term refers to, for example, a mass formed from cells dispersed in a medium such as a culture medium. Cell aggregates include tissues and cell sheets.

The “cell sheet” in the present invention refers to a monolayer or stratified structure composed of one cell or a plurality of cells having a biological junction(s) in at least two-dimensional direction(s). The cell sheet may readily be prepared from adherently cultured cells or a cell aggregate(s) by excision with tweezers, a knife, scissors, or the like.

The “tissue” in the present invention means a structure of cell population having structure in which one kind of cells homogenous in morphology or nature or a plurality of kinds of cells heterogenous in morphology or nature are three-dimensionally arranged in a certain pattern. Examples of the tissue include retinal tissue.

The “retinal tissue” (or “retinal organoid”) in the present invention means a tissue in which one kind or a plurality of kinds of retinal cells constituting one or a plurality of retina layers in the biological retina are three-dimensionally arranged in one or a plurality of layers. Which retinal layer each kind of cells constitutes may be determined by a known method, for example, the presence or absence of expression or the expression level of a cell marker.

The “retinal layer(s)” in the present invention means a layer(s) constituting the retina, and specific examples thereof include the retinal pigment epithelial layer, the photoreceptor cell layer, the outer limiting membrane, the outer nuclear layer, the outer plexiform layer, the internal granular layer, the inner plexiform layer, the ganglion cell layer, the nerve fiber layer, and the inner limiting membrane.

The “photoreceptor cell layer” in the present invention is one of the retinal layers and means the retinal layer comprising many (for example, 70%, preferably 80%, more preferably 90% or more in terms of the number of nuclei of photoreceptor cells present in the photoreceptor cell layer) photoreceptor cells (rod photoreceptor cells, cone photoreceptor cells).

### 1-2. Retinal Cells

The term “retinal cells” in the present invention means cells constituting a retinal layer(s) in the biological retina or progenitor cells thereof. Specific examples thereof include, but are not limited to, photoreceptor cells (rod photoreceptor cells, cone photoreceptor cells), horizontal cells, amacrine cells, interneurons, retinal ganglion cells (ganglion cells), bipolar cells (rod bipolar cells, cone bipolar cells), Muller glial cells, retinal pigment epithelial (RPE) cells, ciliary marginal zone cells, progenitor cells thereof (for example, photoreceptor progenitor cells, bipolar cell progenitor cells), and retinal progenitor cells.

The term “mature retinal cells” means differentiated cells such as photoreceptor cells (rod photoreceptor cells, cone photoreceptor cells), horizontal cells, amacrine cells, interneurons, retinal ganglion cells (ganglion cells), bipolar cells (rod bipolar cells, cone bipolar cells), Muller glial cells, retinal pigment epithelial (RPE) cells, and ciliary marginal zone cells. The term “immature retinal cells” means progenitor cells (for example, photoreceptor progenitor cells, bipolar cell progenitor cells, retinal progenitor cells) determined to differentiate into mature retinal cells.

The photoreceptor progenitor cells, horizontal cell progenitor cells, bipolar cell progenitor cells, amacrine cell progenitor cells, retinal ganglion cell progenitor cells, Muller glial progenitor cells, and retinal pigment epithelial progenitor cells refer to progenitor cells determined to differentiate into photoreceptor cells, horizontal cells, bipolar cells, amacrine cells, retinal ganglion cells, Muller glial cells, and retinal pigment epithelial cells, respectively.

The term “retinal progenitor cells” in the present invention refers to progenitor cells that can differentiate into any of the immature retinal cells such as photoreceptor progenitor cells, horizontal cell progenitor cells, bipolar cell progenitor cells, amacrine cell progenitor cells, retinal ganglion cell progenitor cells, Muller glial progenitor cells, and retinal pigment epithelial progenitor cells and that can finally differentiate into any of mature retinal cells such as photoreceptor cells (rod photoreceptor cells, cone photoreceptor cells), horizontal cells, bipolar cells, amacrine cells, retinal ganglion cells, Muller glial cells, and retinal pigment epithelial cells.

The presence of retinal cells may be determined by the presence or absence of expression of a marker of retinal cells (hereinafter, referred to as a “retinal cell marker”). The presence or absence of expression of a retinal cell marker or the proportion of retinal cell marker-positive cells in a cell population or a tissue may be easily determined by a person skilled in the art, for example, by counting the number of particular retinal cell marker-positive cells by a technique such as flow cytometry using a commercially available antibody and immunostaining and dividing the number by the total number of cells.

Examples of the retinal cell marker include proteins such as Rx (also referred to as “Rax”), PAX6, and Chx10 expressed in retinal progenitor cells, Crx and Blimp1 expressed in photoreceptor progenitor cells, Chx10, PKCα, and L7 expressed in bipolar cells, TuJ1 and Brn3 expressed in retinal ganglion cells, calretinin expressed in amacrine cells, calbindin expressed in horizontal cells, recoverin expressed in mature photoreceptor cells (rod photoreceptor cells and cone photoreceptor cells), Nrl and rhodopsin expressed in rod photoreceptor cells, Rxr-gamma, S-Opsin, and M/L-Opsin expressed in cone photoreceptor cells, GS and GFAP expressed in Muller glial cells, RPE65 and Mitf expressed in retinal pigment epithelial cells, and Rdh10 and SSEA1 expressed in ciliary marginal zone cells.

The term “positive cells” means cells expressing a particular marker on the cell surface or in the cell. For example, the term “Chx10-positive cells” means cells expressing the Chx10 protein in the nucleus.

### 1-3. Bipolar Cell-Regulating Gene

The “bipolar cell-regulating gene” in the present invention refers to a gene expressed in retinal progenitor cells, bipolar cell progenitor cells, and/or bipolar cells and involved in the differentiation, maturation, survival, proliferation, metabolism, or the function of forming the synapse with photoreceptor cells of bipolar cells, but not in the differentiation, maturation, survival, proliferation, metabolism, and function of photoreceptor cells. Therefore, retinal progenitor cells and the like that cause defect of differentiation, degenerative death, dysfunction, and/or the like of bipolar cells, but allow photoreceptor cells to maintain the normal function may be prepared by modifying the bipolar cell-regulating gene. Preferably, the bipolar cell-regulating gene is a gene that is expressed in bipolar cell progenitor cells, and/or bipolar cells, but not expressed in retinal progenitor cells, photoreceptor progenitor cells, or photoreceptor cells.

In one embodiment, the bipolar cell-regulating gene refers to a gene that is expressed in bipolar cell progenitor cells and/or bipolar cells and involved in the maturation of bipolar cells, but not in the differentiation, maturation, survival, proliferation, metabolism, and function of photoreceptor cells. Preferably, the bipolar cell-regulating gene is a gene that is expressed in bipolar cell progenitor cells and/or bipolar cells, but not expressed in retinal progenitor cells, photoreceptor progenitor cells, and photoreceptor cells. The “maturation of bipolar cells” means that bipolar cell progenitor cells differentiate into bipolar cells and become capable of forming the synapse with photoreceptor cells. The maturation of bipolar cells may be confirmed by detecting the expression of the Chx10 protein, the PKCα protein, L7, or the like, which are markers of bipolar cells by a technique such as immunohistological analysis, flow cytometry, or the like.

In one embodiment, the bipolar cell-regulating gene includes a gene encoding a transcription factor involved in the aforementioned function (the differentiation, maturation, survival, proliferation, metabolism, or function of forming the synapse with photoreceptor cells of bipolar cells), specifically a transcription factor that maintains or enhances the function. The bipolar cell-regulating gene is preferably a gene encoding a transcription factor.

The transcription factor contains a region (domain) having a function involved in the regulation of transcription. For example, the transcription factor contains a characteristic DNA binding region (homeodomain, zinc finger domain, basic leucine zipper domain, basic-helix-loop-helix domain, or the like). In one embodiment, the transcription factor further contains a transcription cofactor binding domain. Therefore, those skilled in the art can determine whether a particular gene is a gene encoding a transcription factor or not, for example, based on whether the gene contains a characteristic DNA-binding region or not.

The gene that is expressed in retinal progenitor cells, bipolar cell progenitor cells, and/or bipolar cells can be identified by a person skilled in the art by a well known method. In one embodiment, the gene that is expressed in bipolar cells or retinal progenitor cells may be detected in the fetal stage or the adult stage by gene expression analysis (for example, microarray analysis) and/or histological analysis (for example, immunostaining analysis, in situ hybridization).

Those skilled in the art can identify the gene involved in the differentiation, maturation, survival, proliferation, metabolism, or the function of forming synapse with photoreceptor cells of bipolar cells, but not in the differentiation, maturation, survival, proliferation, metabolism and function of photoreceptor cells by a well known method or known information. In one embodiment, the effect of the aforementioned gene on the differentiation, maturation, survival, proliferation, metabolism or function of forming the synapse with photoreceptor cells of bipolar cells and the differentiation, maturation, survival, proliferation, metabolism, and function of photoreceptor cells may be examined in a genetically modified cell line and/or a genetically modified animals produced by a transfection technique (for example, electroporation, lipofection).

In a specific embodiment, genes expressed in retinal progenitor cells, bipolar cell progenitor cells, or bipolar cells may be screened by gene expression analysis (for example, microarray analysis), and genes involved in the aforementioned function may be selected as candidate genes for the bipolar cell-regulating gene by known information (homology of the genetic sequence to the ISL1 gene or the like, function, conserved functional domain). In a preferred embodiment, genes that are expressed in bipolar cell progenitor cells or bipolar cells, but not in photoreceptor progenitor cells or photoreceptor cells are selected as candidate genes for the bipolar cell-regulating gene. Moreover, in another preferred embodiment, genes encoding transcription factors are selected as candidate genes for the bipolar cell-regulating gene. The genes encoding transcription factors may be selected based on the aforementioned conserved domains or the like.

These methods for selecting candidate genes for the bipolar cell-regulating gene may be used in combination. For example, genes that are expressed in bipolar cells progenitor cells or bipolar cells, but not in photoreceptor progenitor cells or photoreceptor cells and that encode transcription factors may be selected as candidate genes for the bipolar cell-regulating gene.

Furthermore, a gene may be examined whether it is a bipolar cell-regulating gene, for example, by producing retinal tissue from pluripotent stem cells in which a selected gene is modified and performing histologic analysis.

Examples of the bipolar cell-regulating gene include, but are not limited to, genes encoding transcription factors expressed in bipolar cells, such as the ISL1 gene and the BHLHE23 gene, which are involved in the maturation of bipolar cells and genes having nucleotide sequences that are substantially the same as these genes.

The term “genes having nucleotide sequences that are substantially the same” in the present invention means genes that encode proteins having the function that is substantially the same in nature as that of a protein encoded by a gene having a nucleotide sequence set forth in a particular sequence ID number and that have about 80% or more (preferably about 85% or more, about 90% or more, or about 95% or more) sequence identity with the nucleotide sequence set forth in a particular sequence ID number or have nucleotide sequences encoding proteins having about 80% or more (preferably about 85% or more, about 90% or more, or about 95% or more) sequence identity with an amino acid sequence set forth in a particular sequence ID number. The term “genes having nucleotide sequences that are substantially the same” encompasses genes that are an ortholog, a subtype, an isoform, or a mutant and known to have the function that is substantially the same in nature, even if the genes do not satisfy the above definition (for example, sequence identity lower than 80%).

In the present invention, the “function that is substantially the same in nature” means that the nature of the function is qualitatively the same, for example, in a physiological or pharmacological view and quantitative factors such as the degree of the function and the molecular weight of the protein may be different.

The “sequence identity” of a nucleotide sequence or an amino acid sequence in the present invention means proportion (%) of identical nucleotides or amino acids to the total nucleotide sequence or amino acid sequence overlapped in the optimal alignment when 2 nucleotide sequences are aligned using a mathematical algorithm known in the art (preferably, the algorithm is such an algorithm with which the optimal alignment is obtained in consideration of introduction of a gap(s) in one or both sequences.). Those skilled in the art can easily determine the “sequence identity” of a nucleotide sequence or an amino acid sequence. For example, NCBI BLAST (National Center for Biotechnology Information Basic Local Alignment Search Tool) may be used.

The “ortholog” in the present invention means a gene or a protein derived from different animal species having high similarity derived from the same ancestor in the evolutionary tree.

The “subtype” in the present invention means a protein group having high genetic homology and similar function.

The “isoform” in the present invention means a gene or a protein having the function that is substantially the same in nature while the conformation of the protein is different. A plurality of isoforms may be splicing variants derived from the same DNA or they may be derived from different DNAs.

The “mutant” in the present invention refers to a gene that encodes a protein having the function that is substantially the same in nature as that of a protein encoded by a gene having a nucleotide sequence set forth in a particular sequence ID number and that has a nucleotide sequence modified by deletion, addition, insertion or substitution of one or a plurality of nucleotides

Those skilled in the art can determine whether genes are the “genes having nucleotide sequences that are substantially the same” as a particular gene, for example, based on nucleotide sequences of genes listed in ncbi.nlm.nih.gov.

As used herein, the “gene” means a nucleotide sequence of a region encoding a particular protein (a region that contains from an initiation codon to a termination codon and may contain an intron(s)) and its flanking regions (regions such as a promoter, an enhancer, a silencer, and a terminator) present on a chromosome. The ranges of the flanking regions vary depending on the gene, and examples thereof include 50 bp, 100 bp, 200 bp, 300 bp, 400 bp, 500 bp, 1000 bp, and 2000 bp. Accordingly, the modification of a gene includes not only the modification of the region encoding a particular protein, but also the modification of its flanking regions.

The ISL1 gene and the BHLHE23 gene will be described below.

The ISL1 (Insulin gene enhancer protein 1, ISL LIM homeobox 1, Islet-1) gene is a known gene. Examples of the ISL1 gene include genes having the nucleotide sequence set forth in SEQ ID NO: 1 (GenBank Accession Gene ID: 3670 (NC 000005.10)) or nucleotide sequences that are substantially the same as this nucleotide sequence. Moreover, the ISL1 gene also encompasses genes containing, as exon, nucleotide sequences containing an exon part contained in the nucleotide sequence set forth in SEQ ID NO: 1 (for example, the nucleotide sequence set forth in SEQ ID NO: 2 (GenBank Accession Gene ID: NM_002202.2)) or nucleotide sequences that are substantially the same as this exon part. The ISL1 protein encoded by a nucleotide sequence corresponding to the positions 549 to 1598 in the nucleotide sequence set forth in SEQ ID NO: 2 has the amino acid sequence set forth in SEQ ID NO: 3 (GenBank Accession Gene ID: NP_002193.2). The ISL1 gene also encompasses genes containing, as exon, nucleotide sequences encoding the amino acid sequence set forth in SEQ ID NO: 3 or amino acid sequences that are substantially the same as this.

A known ortholog of the human ISL1 gene is the murine ISL1 gene having the nucleotide sequence set forth in SEQ ID NO: 4 (GenBank Accession Gene ID: 16392 (NC_000079.6)). The ISL1 gene also encompasses genes having the nucleotide sequence set forth in SEQ ID NO: 4 or nucleotide sequences that are substantially the same as this. Moreover, the ISL1 gene also encompasses genes containing, as exon, nucleotide sequences containing an exon part contained in the nucleotide sequence set forth in SEQ ID NO: 4 (for example, the nucleotide sequence set forth in SEQ ID NO: 5 (GenBank Accession Gene ID: NM_021459.4)) or nucleotide sequences that are substantially the same as this. The ISL1 protein encoded by a nucleotide sequence corresponding to the positions 267 to 1316 in the nucleotide sequence set forth in SEQ ID NO: 5 has the amino acid sequence set forth in SEQ ID NO: 6 (GenBank Accession Gene ID: NP_067434.3). The ISL1 gene also encompasses genes containing, as exon, nucleotide sequences encoding the amino acid sequence set forth in SEQ ID NO: 6 or amino acid sequences that are substantially the same as this.

A known human subtype of the human ISL1 gene is the human ISL2 gene having the nucleotide sequence set forth in SEQ ID NO: 7 (GenBank Accession Gene ID: 64843 (NC_000015.10)). The ISL1 gene also encompasses genes having the nucleotide sequence set forth in SEQ ID NO: 7 or nucleotide sequences that are substantially the same as this. Moreover, the ISL1 gene also encompasses genes containing, as exon, nucleotide sequences containing an exon part contained in the nucleotide sequence set forth in SEQ ID NO: 7 (for example, the nucleotide sequence set forth in SEQ ID NO: 8 (GenBank Accession Gene ID: NM_145805)) or nucleotide sequences that are substantially the same as this. The ISL1 protein encoded by a nucleotide sequence corresponding to the positions 161 to 1240 in the nucleotide sequence set forth in SEQ ID NO: 8 has the amino acid sequence set forth in SEQ ID NO: 9 (GenBank Accession Gene ID: NP_665804.1). The ISL1 gene also encompasses genes containing, as exon, nucleotide sequences encoding the amino acid sequence set forth in SEQ ID NO: 9 or amino acid sequences that are substantially the same as this.

The term “genes having nucleotide sequences that are substantially the same as SEQ ID NO: 1” means genes that encode proteins having the function that is substantially the same in nature as that of a protein having the amino acid sequence set forth in SEQ ID No: 3 wherein the genes have about 80% or more (preferably about 85% or more, about 90% or more, or about 95% or more) sequence identity with a nucleotide sequence set forth in SEQ ID NO: 1 or wherein the exon of the genes have such sequence identity with the nucleotide sequence set forth in SEQ ID NO: 2, or genes that contain a nucleotide sequence encoding a protein having about 80% or more (preferably about 85% or more, about 90% or more, or about 95% or more) sequence identity with the amino acid sequence set forth in SEQ ID NO: 3.

The term “genes having nucleotide sequences that are substantially the same as SEQ ID NO: 4” means genes that encode proteins having the function that is substantially the same in nature as that of a protein having the amino acid sequence set forth in SEQ ID No: 6 wherein the genes that have about 80% or more (preferably about 85% or more, about 90% or more, or about 95% or more) sequence identity with a nucleotide sequence set forth in SEQ ID NO: 4 or wherein the exon of the genes have such sequence identity with the nucleotide sequence set forth in SEQ ID NO: 5, or genes that contain a nucleotide sequence encoding a protein having about 80% or more (preferably about 85% or more, about 90% or more, or about 95% or more) sequence identity with the amino acid sequence set forth in SEQ ID NO: 6.

The term “genes having nucleotide sequences that are substantially the same as SEQ ID NO: 7” means genes that encode proteins having the function that is substantially the same in nature as that of a protein having the amino acid sequence set forth in SEQ ID No: 9 wherein the genes have about 80% or more (preferably about 85% or more, about 90% or more, or about 95% or more) sequence identity with a nucleotide sequence set forth in SEQ ID NO: 7 or wherein the exon of the genes have such sequence identity with the nucleotide sequence set forth in SEQ ID NO: 8, or genes that contain a nucleotide sequence encoding a protein having about 80% or more (preferably about 85% or more, about 90% or more, or about 95% or more) sequence identity with the amino acid sequence set forth in SEQ ID NO: 9.

It has been reported that the ISL1 protein is a homeodomain transcription factor, has two LIM domains, LIM1 and LIM2, an Lhx3 binding domain, and a DNA binding domain, and is involved in the maturation of bipolar cells.

Therefore, “having the function that is substantially the same in nature as that of a protein having the amino acid sequence set forth in SEQ ID No: 3” means having an ability to bind with DNA and the function of positively or negatively regulating transcription of a gene.

Specific examples of the “gene having a nucleotide sequence that is substantially the same as the nucleotide sequence set forth in SEQ ID NO: 1, 4, or 7”, that is, the ISL1 gene include the following (1) or (2):
(1) a nucleotide sequence set forth in SEQ ID NO: 1, 4, or 7;
(2) a nucleotide sequence modified from the nucleotide sequence set forth in SEQ ID NO: 1, 4, or 7 by deletion, addition, insertion, or substitution of one or a plurality of nucleotides, and encoding a protein having 80% or more sequence identity with the amino acid sequence set forth in SEQ ID NO: 3, 6, or 9, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 3, 6, or 9.

Here, examples of the “nucleotide sequence modified by deletion, addition, insertion or substitution of one or a plurality of nucleotides” include nucleotide sequences that have, after the deletion, addition, insertion or substitution, 80% or more, 85% or more, 90% or more, 95% or more, 97% or more, 98% or more, or 99% or more sequence identity with the nucleotide sequence before the deletion, addition, insertion, or substitution. Examples of the specific number of nucleotides in the “one or a plurality of nucleotides” are 1 to 100, 1 to 50, 1 to 30, 1 to 10, and 1 to several (2, 3, 4, or 5). The deletion, addition, insertion, or substitution may be a combination thereof.

In one embodiment of the present invention, the ISL1 gene is a gene at a chromosomal position “5q11.1” or “15q24.3” when it is a human gene or at a chromosomal position “13D2.3” when it is a murine gene. Those skilled in the art can determine the chromosome position of the ISL1 gene (ortholog) in other animal species based, for example, on nucleotide sequences of genes listed in ncbi.nlm.nih.gov.

The ISL1 gene is preferably the human ISL1 gene or the murine ISL1 gene.

The BHLHE23 (basic helix-loop-helix family, member e23) gene is a known gene. Examples of the BHLHE23 gene include genes having the nucleotide sequence set forth in SEQ ID NO: 10 (GenBank Accession Gene ID: 128408 (NC_000020.11)) or nucleotide sequences that are substantially the same as the nucleotide sequence. Moreover, the BHLHE23 gene also encompasses genes containing, as exon, nucleotide sequences containing an exon part contained in the nucleotide sequence set forth in SEQ ID NO: 10 (for example, the nucleotide sequence set forth in SEQ ID NO: 11 (GenBank Accession Gene ID: NM_080606)) or nucleotide sequences that are substantially the same as this. The BHLHE23 protein encoded by the nucleotide positions 262 to 987 in the nucleotide sequence set forth in SEQ ID NO: 11 is a protein having the amino acid sequence set forth in SEQ ID NO: 12 (GenBank Accession Gene ID: NP_542173.2). The BHLHE23 gene also encompasses genes containing, as exon, nucleotide sequences encoding the amino acid sequence set forth in SEQ ID NO: 12 or amino acid sequences that are substantially the same as this.

Known ortholog of the human BHLHE23 gene is the murine BHLHE23 gene (Bhlhb4 gene) having the nucleotide sequence set forth in SEQ ID NO: 13 (GenBank Accession Gene ID: 140489 (NC_000068.7)). The BHLHE23 gene encompasses genes having the nucleotide sequence set forth in SEQ ID NO: 13 or nucleotide sequences that are substantially the same as this. Moreover, the BHLHE23 gene also encompasses genes containing, as exon, nucleotide sequences containing an exon part contained in the nucleotide sequence set forth in SEQ ID NO: 13 (for example, the nucleotide sequence set forth in SEQ ID NO: 14 (GenBank Accession Gene ID: NM_080641.5)) or nucleotide sequences that are substantially the same as this. The murine Bhlhb4 protein encoded by the nucleotide positions 158 to 829 in the nucleotide sequence set forth in SEQ ID NO: 14 has an amino acid sequence set forth in SEQ ID NO: 15 (GenBank Accession Gene ID: NP_542372.2). The BHLHE23 gene also encompasses genes containing, as exon, nucleotide sequences encoding the amino acid sequence set forth in SEQ ID NO: 15 or amino acid sequences that are substantially the same as this.

The term “genes having nucleotide sequences that are substantially the same as SEQ ID NO: 10” means genes that encode proteins having the function that is substantially the same in nature as that of a protein having the amino acid sequence set forth in SEQ ID No: 12 wherein the genes have about 80% or more (preferably about 85% or more, about 90% or more, or about 95% or more) sequence identity with a nucleotide sequence set forth in SEQ ID NO: 10 or wherein the exon of the genes have such sequence identity with the nucleotide sequence set forth in SEQ ID NO: 11, or genes that contain a nucleotide sequence encoding a protein having about 80% or more (preferably about 85% or more, about 90% or more, or about 95% or more) sequence identity with the amino acid sequence set forth in SEQ ID NO: 12.

The term “genes having nucleotide sequences that are substantially the same as SEQ ID NO: 13” means genes that encode proteins having the function that is substantially the same in nature as that of a protein having the amino acid sequence set forth in SEQ ID No: 15 wherein the genes have about 80% or more (preferably about 85% or more, about 90% or more, or about 95% or more) sequence identity with the nucleotide sequence set forth in SEQ ID NO: 13 or wherein the exon of the genes have such sequence identity with the nucleotide sequence set forth in SEQ ID NO: 14, or genes that contain a nucleotide sequence encoding a protein having about 80% or more (preferably about 85% or more, about 90% or more, or about 95% or more) sequence identity with the amino acid sequence set forth in SEQ ID NO: 15.

It has been reported that the BHLHE23 protein is a Basic helix-loop-helix transcription factor, has a DNA binding domain and the helix-loop-helix domain, and is involved in the maturation of bipolar cells.

Therefore, “having the function that is substantially the same in nature with a protein having the amino acid sequence set forth in SEQ ID NO: 12” means having an ability to bind with DNA and the function of positively or negatively regulating transcription of a gene.

Specific examples of the “gene having a nucleotide sequence that is substantially the same as the nucleotide sequence set forth in SEQ ID NO: 10 or 13”, that is, the BHLHE23 gene include the following (1) or (2):
(1) a nucleotide sequence set forth in SEQ ID NO: 10 or 13;
(2) a nucleotide sequence modified from the nucleotide sequence set forth in SEQ ID NO: 10 or 13 by deletion, addition, insertion or substitution of one or a plurality of nucleotides, and encoding a protein having 80% or more sequence identity with an amino acid sequence set forth in SEQ ID NO: 12 or 15, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 12 or 15.

Here, examples of the “nucleotide sequence modified by deletion, addition, insertion or substitution of one or a plurality of nucleotides” include nucleotide sequences that have, after the deletion, addition, insertion or substitution, 80% or more, 85% or more, 90% or more, 95% or more, 97% or more, 98% or more, or 99% or more sequence identity with the nucleotide sequence before the deletion, addition, insertion, or substitution. Examples of the specific number of nucleotides in the “one or a plurality of nucleotides” are 1 to 100, 1 to 50, 1 to 30, 1 to 10, and 1 to several (2, 3, 4, or 5). The deletion, addition, insertion, or substitution may be a combination thereof.

In one embodiment of the present invention, the BHLHE23 gene is a gene at a human chromosomal position “20q13.33” or at a murine chromosomal position “2103.34 cM”. Those skilled in the art can determine the chromosome position of the BHLHE23 gene (ortholog) in other animal species based, for example, on nucleotide sequences of genes listed in http://www.ncbi.nlm.nih.gov.

The BHLHE23 gene is preferably the human BHLHE23 gene or the murine BHLHE23 gene.

### 1-4. Genetic Modification

The “genetic modification” and the “modification of a gene” in the present invention mean elimination or attenuation of the expression or function of the mRNA or protein encoded by a particular gene by addition, insertion, deletion, substitution, or the like of one or a plurality of nucleotides to the particular gene. Accordingly, the genetic modification includes deletion of a gene(s). The number and the positions of nucleotides (including an enhancer(s), a promoter(s), an intron(s), and the like) to be added, inserted, deleted, substituted, or so and the method of the genetic modification are not limited, as long as the expression or function of the protein encoded by the particular gene is eliminated or attenuated.

In one embodiment of the present invention, the modification of the bipolar cell-regulating gene also includes substitution of a sequence encoding a protein (the exon and the intron) downstream of the enhancer and the promoter in the bipolar cell-regulating gene (for example, the ISL1 gene or the BHLHE23 gene) with a special sequence, while maintaining the enhancer and the promoter. Examples of the special sequence include suicide genes, survival regulatory genes, growth regulatory genes, differentiation regulatory genes, and metabolism regulatory genes, (hereinafter, also referred to as the “suicide genes and the like”), or genes encoding microRNAs that can suppress the expression of the bipolar cell-regulating gene, antisense RNAs containing nucleic acid sequences identical or homologous with the bipolar cell-regulating gene, and noncoding RNAs of the bipolar cell-regulating gene (hereinafter, also referred to as the “microRNA genes and the like”), or the like. Examples of the suicide genes and the like include genes for apoptosis inducing factors and the like. Examples of the microRNA genes and the like include genes encoding the microRNAs and the like against other bipolar cell-regulating genes.

By expressing these genes under the promoter of a bipolar cell-regulating gene, for example, the induction of bipolar cell-specific cell death or the suppression of expression of a bipolar cell-regulating gene other than the replaced gene may be performed. Thus, the bipolar cells may be lead to cell death by the expression of one of the suicide genes and the like that has been incorporated downstream of the same enhancer and promoter at the time when the bipolar cell-regulating gene is expressed since the bipolar cell-regulating gene is expressed by the process inducing the bipolar cells. Moreover, the function of mRNA of the bipolar cell-regulating gene may be suppressed by expressing one of the microRNA genes and the like.

The special sequence may be substituted for the internal bipolar cell-regulating gene itself in pluripotent stem cells or may be introduced downstream of the enhancer and the promoter of the bipolar cell-regulating gene in the genome of pluripotent stem cells while maintaining the bipolar cell-regulating gene. In this case, the position of the introduction is not particularly limited, as long as it is a position at which the special sequence can function. Preferably, the special sequence is introduced, without substituting the bipolar cell-regulating gene itself.

In one embodiment, examples of the genetic modification include the genome editing that substantially eliminates the function from the gene. Examples of the “genome editing that substantially eliminates the function” include introduction of a mutation that modifies the gene into a mutated form and eliminates the function of the wild type (for example, a nonsense mutation such as frameshift), genome editing that decreases the expression level of the gene, and the like.

In one embodiment, examples of the “genome editing that decreases the expression level of the gene” include modification of an expression level regulatory sequence for a gene in genome, and the like. Examples of the expression level regulatory sequence include an enhancer and/or a promoter. For example, the enhancer and/or promoter is present in the upstream sequence, in the downstream sequence, and/or within the gene (for example, an intronic region) of the bipolar cell-regulating gene.

The terms “gene deletion” and “deletion of gene” in the present invention mean eliminating or attenuating, by deleting a nucleotide(s) from a particular gene, the expression or function of the protein encoded by the particular gene. The number and the positions of the nucleotides (including an enhancer(s), a promoter(s), an intron(s), and the like) and the method of the gene deletion are not limited, as long as the expression or function of the protein encoded by the particular gene is eliminated or attenuated. For example, each of the murine and human ISL1 genes may be deleted by deleting the first and second exons and the murine Bhlhb4 gene may be deleted by deleting a flanking sequence (for example, about 150 nucleotides) including the initiation codon of the protein coding region. For example, such a gene may be deleted by deleting some exons (for example, the first or second exon) or by deleting a plurality of nucleotides (1 to the total number of nucleotides in the protein coding region, for example, 10 to 500 nucleotides, 100 to 300 nucleotides, or about 150 nucleotides) including the initiation codon of the protein coding region.

In the present invention, genetic modification may be performed in a somatic cell before reprograming, a pluripotent stem cell, or a retinal cell. Examples of the method for obtaining a genetically modified retinal cell include a method involving inducing the differentiation of a genetically modified pluripotent stem cell (including a pluripotent stem cell obtained by reprograming a genetically modified somatic cell) and a method involving performing genetic modification in a retinal cell. Preferably, the genetic modification is performed in a pluripotent stem cell.

The “genetically modified pluripotent stem cell” in the present invention means a pluripotent stem cell in which genetic modification has been performed, within the extent that allows maintenance of pluripotency. In one embodiment, the “genetically modified pluripotent stem cell” means a pluripotent stem cell in which genetic modification has been performed within the extent that allows maintenance of pluripotency and proliferative capacity (self-renewal capacity).

In the present invention, the gene to be modified is the bipolar cell-regulating gene, but other genes may be further modified. One embodiment includes, but is not limited to, substitution of a particular cell marker gene with a gene encoding a fluorescence protein for the purpose of confirming the existence of the particular cell, and the like.

The genetically modified pluripotent stem cell or retinal cell may be produced, for example, by using a homologous recombination technique. The modification of a target gene on a chromosome may be performed by using a method described in Manipulating the Mouse Embryo, A Laboratory Manual, Second Edition, Cold Spring Harbor Laboratory Press (1994); Gene Targeting, A Practical Approach, IRL Press at Oxford University Press (1993); Biomanual series 8, Gene targeting, Production of mutant mouse using embryonic stem cells, Yodosha Company, Ltd. (1995), and the like.

In a specific example, genomic DNA containing a target gene to be modified is isolated and a targeting vector for homologously recombining the target gene is prepared using isolated genomic DNA. By introducing the prepared targeting vector into a stem cell and selecting a cell in which homologous recombination between the target gene and the targeting vector has occurred, a stem cell in which the chromosomal gene has been modified may be prepared.

Examples of the method for isolating genomic DNA containing the target gene include known methods described in Molecular Cloning, A Laboratory Manual, Second Edition, Cold Spring Harbor Laboratory Press (1989) and Current Protocols in Molecular Biology, John Wiley & Sons (1987-1997). Genomic DNA containing the target gene may be isolated by using a genomic DNA library screening system (manufactured by Genome Systems), Universal Genome Walker Kits (manufactured by CLONTECH), or the like. Instead of using genomic DNA, a polynucleotide encoding the target protein may be used. Such a polynucleotide may be obtained by amplifying an applicable polynucleotide by PCR.

The preparation of the targeting vector for homologously recombining the target gene and effective selection of homologous recombinants may be performed according to a method described in Gene Targeting, A Practical Approach, IRL Press at Oxford University Press (1993); Biomanual series 8, Gene targeting, Production of mutant mouse using embryonic stem cells, Yodosha Company, Ltd. (1995), and the like. The targeting vector either of a replacement type or an insertion type may be used. Examples of the method for selection include methods such as positive selection, promoter selection, negative selection, or poly A selection.

Examples of the method for selecting homologous recombinants of interest from selected cell lines include Southern hybridization with genomic DNA, PCR, and the like.

Moreover, the genetically modified pluripotent stem cell or retinal cell may be produced by genome editing. The genome editing is a genetic modification technique for gene-specific disruption, knock-in of a reporter gene, or the like by a technique such as the Zinc Finger system, the CRISPR/Cas9 system, and Transcription Activator-Like Effector Nucleases (TALEN).

In the Zinc Finger system, the target gene is usually recognized and cleaved by using artificial chimeric proteins composed of a domain referred to as the zinc finger motif that specifically binds to DNA and the restriction enzyme FokI. When the two artificial chimeric proteins have bound to the closely positioned target sequence, the DNA cleavage domains form a dimer to cleave DNA. The cleaved DNA is repaired by homologous recombination or non-homologous end-joining and the gene of interest is modified at the same time.

In the CRISPR/Cas9 genome editing system, an expression vector or mRNA of Cas9 (a DNA cleavage enzyme) or the Cas9 protein and an expression vector that expresses a guide RNA under the control of the polymerase III promoter or the like or the guide RNA itself are usually introduced into a cell. The guide RNA may be a fusion RNA of an RNA (crRNA) complementary to the target genome sequence and of tracrRNA. When a protospacer adjacent motif (PAM-sequence NGG) is present at the 3′ end of the target genome sequence, Cas9 dissociates the DNA double strand, the target sequence is recognized by the guide RNA, and the both strands are cleaved. A mutation is introduced in the process of repairing the cleaved site.

The transcription activator-like effector nuclease (TALEN) is a system using the TAL effector, which the phytopathogenic bacterium Xanthomonas spp. produces. TALEN is usually an artificial nuclease that is a fusion of a DNA binding domain from the TAL effector and a DNA cleavage domain from the FokI nuclease. The DNA binding domain is composed of repeats of a 34 amino acid residue sequence and one repeat recognizes one nucleotide in the target DNA. The 12th and 13th amino acid residues in the repeat sequence are referred to as the repeat variable di-residues (RVD) and the sequence of them determines the target nucleotide. By designing TALEN such that 2 sets of TALEN molecules face each other on a particular sequence in the genome, the FokI domains dimerize on the target sequence and the TALEN pair exhibits the nuclease activity. The cleaved DNA double-strand is repaired by an intrinsic cellular mechanism and a mutation is introduced in the process. When genome editing is performed by TALEN, an expression vector or mRNA of TALEN is introduced into cells.

### 1-5. Pluripotent Stem Cell

In the present invention, the term “stem cells” means undifferentiated cells having differentiation potency and capacity to proliferate (in particular self-renewal capacity) while maintaining differentiation potency. The stem cells include subpopulations such as pluripotent stem cells, multipotent stem cells, and unipotent stem cells depending on the differentiation potency. The term “pluripotent stem cells” refers to stem cells that can be cultured in vitro and have the ability to differentiate to all the cell lineages belonging to the 3 blastoderms (the ectoderm, the mesoderm, the endoderm) and/or extraembryonic tissue (pluripotency). The term “multipotent stem cells” means stem cells having the ability to differentiate into a plurality, but not all, of tissues or cells. The term “unipotent stem cells” means stem cells having the ability to differentiate into a particular tissue or cell.

The pluripotent stem cells may be induced from fertilized eggs, clonal embryos, germline stem cells, tissue stem cells, somatic cells, and the like. Examples of the pluripotent stem cells include embryonic stem cells (ES cells), EG cells (Embryonic germ cells), induced pluripotent stem cells (iPS cells), and the like. The pluripotent stem cells also encompass Muse cells (Multi-lineage differentiating stress enduring cells) obtained by mesenchymal stem cells (MSCs) and GS cells prepared from germ cells (for example, the testis). The embryonic stem cells are established in 1981 for the first time and have also been applied to the production of knockout mice since 1989. Human embryonic stem cells were established in 1998 and have been used in regenerative medicine. The embryonic stem cells may be produced by culturing the internal cell mass with feeder cells or in a culture medium containing LIF (leukemia inhibitory factor). Methods for producing embryonic stem cells are described in, for example, WO96/22362, WO02/101057, U.S. Pat. Nos. 5,843,780, 6,200,806, 6,280,718, and the like. The embryonic stem cells are available from certain institutions and commercial products may also be purchased. For example, the human embryonic stem cells KhES-1, KhES-2, and KhES-3 are available from the Institute for Frontier Medical Sciences, Kyoto University. The human embryonic stem cell line Rx::Venus (derived from the cell line KhES-1) is available from the Institute of Physical and Chemical Research. The murine embryonic stem cell lines EB5 and D3 are respectively available from the Institute of Physical and Chemical Research and ATCC.

The nuclear transplantation embryonic stem cells (ntES cells), which are one of the embryonic stem cells, may be established from clonal embryos produced by transplanting a somatic nucleus to an ovum from which the nucleus is removed.

The EG cells may be produced by culturing primordial germ cells in a culture medium containing mSCF, LIF, and bFGF (Cell, 70:841-847, 1992).

The “induced pluripotent stem cells” in the present invention are cells in which pluripotency is induced by reprogramming somatic cells by a known method. Specific examples thereof include cells obtained by reprogramming differentiated somatic cells such as a fibroblasts or peripheral blood mononuclear cells by expressing a combination of a plurality of genes selected from reprogramming genes including Oct3/4, Sox2, Klf4, Myc (c-Myc, N-Myc, L-Myc), Glis1, Nanog, Sall4, Lin28, and Esrrb to induce pluripotency. Examples of preferred combinations of reprogramming factors include (1) Oct3/4, Sox2, Klf4, and Myc (c-Myc or L-Myc), and (2) Oct3/4, Sox2, Klf4, Lin28, and L-Myc (Stem Cells, 2013, 31:458-466).

The induced pluripotent stem cells were established from murine cells by Yamanaka et al. in 2006 (Cell, 2006, 126 (4), pp. 663-676). The induced pluripotent stem cells were also established from human fibroblasts in 2007 and have pluripotency and self-renewal capacity like embryonic stem cells (Cell, 2007, 131 (5), pp. 861-872, Science, 2007, 318 (5858), pp. 1917-1920, Nat. Biotechnol., 2008, 26 (1), pp. 101-106).

The induced pluripotent stem cells may be induced from somatic cells by the addition of compounds, other than by a method of production by direct reprogramming by gene expression (Science, 2013, 341, pp. 651-654).

Moreover, established induced pluripotent stem cells are available and, for example, human induced pluripotent cell lines such as 201B7 cells, 201B7-Ff cells, 253G1 cells, 253G4 cells, 1201C1 cells, 1205D1 cells, 1210B2 cells, 1231A3 cells, and the like established in Kyoto University are available from Kyoto University. As Examples of the established induced pluripotent stem cells, Ff-101 cells and Ff-114 cells established at Kyoto University are available from Kyoto University.

Examples of the somatic cells to be used in the production of induced pluripotent stem cells include, but are not particularly limited to, fibroblasts, hematopoietic cells (for example, peripheral blood mononuclear cells (PBMCs), T cells), hepatocytes, pancreatic cells, intestinal epithelium cells, smooth muscle cells derived from tissue, and the like.

When reprogramming is performed by expression of several genes in the production of induced pluripotent stem cells, the means for expressing the genes is not particularly limited. Examples of the means include infection using a virus vector (for example, a retroviral vector, a lentiviral vector, a Sendai virus vector, an adenovirus vector, an adenoassociated virus vector), transfection (for example, the calcium phosphate method, lipofection, the RetroNectin method, electroporation) using a plasmid vector (for example, a plasmid vector, an episomal vector), transfection using an RNA vector (for example, the calcium phosphate method, lipofection, electroporation), direct injection of a protein (for example, a method using a needle, lipofection, electroporation), and the like.

The induced pluripotent stem cells may be produced in the presence of feeder cells or in the absence of feeder cells (feeder-free). In the production of the induced pluripotent stem cells in the presence of feeder cells, the induced pluripotent stem cells may be produced in the presence of an undifferentiated state-maintaining factor by a known method. The culture medium to be used in the production of induced pluripotent stem cells in the absence of feeder cells is not particularly limited, but examples thereof include maintenance culture media for known embryonic stem cells and/or induced pluripotent stem cells or culture media for establishing induced pluripotent stem cells in feeder-free conditions. Examples of the culture media for establishing induced pluripotent stem cells in feeder-free conditions include feeder-free culture medium such as the Essential 8 medium (E8 medium), the Essential 6 medium, the TeSR medium, the mTeSR medium, the mTeSR-E8 medium, the Stabilized Essential 8 medium, and the StemFit medium. In the production of induced pluripotent stem cells, the induced pluripotent stem cells may be produced, for example, by genetically introducing the 4 factors: Oct3/4, Sox2, Klf4, and Myc into somatic cells in feeder-free conditions using a Sendai virus vector.

The pluripotent stem cells to be used in the present invention are preferably embryonic stem cells or induced pluripotent stem cells and more preferably induced pluripotent stem cells.

The pluripotent stem cells to be used in the present invention are mammalian pluripotent stem cells, preferably rodent (for example, mouse or rat) or primate (for example, human or monkey) pluripotent stem cells, more preferably human or murine pluripotent stem cells, and further preferably human iPS cells or human ES cells.

Examples of the multipotent stem cells include tissue stem cells (referred to as tissue stem cells, tissue-specific stem cells, or somatic stem cells) such as hematopoietic stem cells, neural stem cells, retinal stem cells, and mesenchymal stem cells.

### 2. Regarding Culture of Cell Population for Transplant

One embodiment of the present invention provides a culture of a cell population for transplant, comprising: (1) the cell population for transplant and (2) a medium necessary to maintain viability of the cell population for transplant.

The “culture” in the present invention means a liquid that contains a medium necessary for maintaining viability and a cell population and may further contain a biological substance added or produced by the cell population. Examples of the biological substance include, but are not limited to, cytokines, chemokines, and the like.

Examples of the “medium necessary for maintaining viability” in the present invention include a culture medium and a physiological buffer solution, and the like, but the medium is not particularly limited as long as the cell population containing retinal cells such as retinal progenitor cells survives and those skilled in the art may select such a medium as appropriate. Examples thereof include culture media modified from basal media usually used for culture of animal cells. Examples of the basal media include culture media that may be used in culture of animal cells such as BME medium, BGJb medium, 1066 CMRL medium, Glasgow MEM (GMEM) medium, Improved MEM Zinc Option medium, IMDM medium, 199 Medium, Eagle MEM medium, αMEM medium, DMEM medium, F-12 medium, DMEM/F12 medium, IMDM/F12 medium, Ham's medium, RPMI 1640 medium, Fischer's medium, or mixed culture media thereof.

### 3. Regarding Method for Producing Cell Population for Transplant Containing Retinal Cell

One embodiment of the present invention is a method for producing a cell population for transplant comprising retinal cells, comprising the following steps (1) and (2):
(1) modifying a bipolar cell-regulating gene of pluripotent stem cells to obtain in vitro, a cell population comprising the pluripotent stem cells with the modified bipolar cell-regulating gene;
(2) inducing differentiation of the cell population comprising the pluripotent stem cells obtained in step (1) into retinal cells in vitro to obtain a cell population for transplant comprising the retinal cells.

As used herein, “modifying a bipolar cell-regulating gene of a pluripotent stem cells” includes an embodiment involving modifying a bipolar cell-regulating gene in a somatic cell before reprogramming, and then producing a pluripotent stem cell having a modified bipolar cell-regulating gene by reprograming the somatic cell before reprogramming.

Those skilled in the art may store the cell population obtained in step (1) by a well known method. The stored cell population is a raw material or a production intermediate for obtaining retinal cells with a modified bipolar cell-regulating gene. One embodiment of the present invention provides the raw material or production intermediate of retinal cells with a modified bipolar cell-regulating gene.

Examples of the method for storing the cell population obtained in step (1) include cryopreservation. The method for cryopreservation is not particularly limited, as long as it is a method generally known as a method for cryopreserving cells. For example, the cell population obtained in step (1) may be suspended in a culture medium containing a cryoprotective agent such as DMSO or glycerin and cryopreserved. Moreover, a commercially available cell cryopreservation medium ‘StemCellBanker (Nippon Zenyaku Kogyo Co., Ltd., registered trademark)” may also be used. The long-term preservation of cell population is possible by cryopreservation.

One embodiment of the present invention provides, as a raw material or a production intermediate for obtaining retinal cells with a modified bipolar cell-regulating, a master cell bank or a working cell bank of cell population containing a pluripotent stem cell with a modified bipolar cell-regulating gene.

More specifically, it is possible to produce the master cell bank by expanding a cell obtained in step (1) and then cryopreserving the resulting cells. Furthermore, the working cell bank may be produced by expanding a cell thawed from the master cell bank. For producing the master cell bank and the working cell bank, the smaller passage number of cells is preferred.

Here, the “master cell bank” refers to a preparation obtained by expanding a seed cell line that serves as an origin of all cell seeds for production and dispensing the resulting cells into a plurality of ampules. Moreover, the “working cell bank” refers to a preparation obtained by expanding one or a plurality of cells reactivated from the master cell bank and dispensing the resulting cells into a plurality of ampules.

One embodiment of the present invention provides a method for producing a raw material or a production intermediate for retinal cells with a modified bipolar cell-regulating gene (for example, a master cell bank of a cell population(s) containing a pluripotent stem cells with a modified bipolar cell-regulating gene), comprising the following steps (i) to (iii):
(i) modifying a bipolar cell-regulating gene of a pluripotent stem cells to obtain in vitro, a cell population of the pluripotent stem cell with a modified bipolar cell-regulating gene;
(ii) proliferating the cell population of the pluripotent stem cell obtained in step (i) by culture,
(iii) cryopreserving the cell population of the pluripotent stem cell obtained in step (ii).

It is possible to provide the cryopreserved cell population (including the master cell bank or working cell bank) of pluripotent stem cells with a modified bipolar cell-regulating gene to step (2) after thawing. Those skilled in the art may thaw frozen cells by a well known method. An unfrozen or frozen or thawed-after-freezing cell population of pluripotent stem cell with a modified bipolar cell-regulating gene is also one embodiment of the present invention.

Accordingly, another one embodiment of the present invention is a method for producing a cell population comprising retinal cells, comprising the following steps (iv) and (v):
(iv) thawing a cryopreserved cell population of pluripotent stem cell with a modified bipolar cell-regulating gene,
(v) inducing differentiation of the cell population comprising the pluripotent stem cells obtained in step (iv) into the retinal cells in vitro to obtain a cell population for transplant comprising the retinal cells.

Step (1) and (2) will be described in detail below.

### 3-1. Regarding Step (1)

Preferable examples of the pluripotent stem cells in step (1) include iPS cells or ES cells.

Here, the method for producing the iPS cells or ES cells is not particularly limited and the iPS cells or ES cells may be produced by a method well known to those skilled in the art as described above. Preferably, the production is conducted in the absence of feeder cells (feeder-free).

In the present invention, “the production is conducted in the absence of feeder cells (feeder-free)” means culturing in the absence of feeder cells. Examples of being in the absence of feeder cells include conditions in which feeder cells are not added or conditions in which feeder cells are substantially absent (for example, the proportion of the number of feeder cells to the total number of cells is 3% or less and preferably 0.5% or less).

In step (1), the maintenance culture or expansion of pluripotent stem cells may be conducted depending on need. The maintenance culture or expansion may be conducted by a method well known to those skilled in the art, but preferably conducted feeder-free.

The culture medium used in the maintenance culture or expansion of the pluripotent stem cells is not limited as long as the maintenance culture or expansion is possible. Under feeder-free conditions, a culture medium containing an undifferentiated state-maintaining factor for enabling undifferentiated state-maintenance culture is used, and many synthetic culture media have been developed and marketed, which may be used. Examples thereof include Essential 8 (manufactured by Life Technologies) medium, S-medium (manufactured by DS Pharma Biomedical Co., Ltd.), StemPro (manufactured by Life Technologies, registered trademark), hESF9 (Proc Natl Acad Sci USA. 2008 September 9, 105 (36): 13409-14), mTeSR1 (manufactured by STEMCELL Technologies Inc.), mTeSR2 (manufactured by STEMCELL Technologies Inc.), TeSR-E8 (manufactured by STEMCELL Technologies Inc.), and StemFit (manufactured by Ajinomoto Co., Inc., registered trademark).

The genetic modification in step (1) may be conducted by a method well known to those skilled in the art as described above.

Those skilled in the art can determine how to modify the bipolar cell-regulating gene for eliminating or attenuating the expression or function of the bipolar cell-regulating gene.

One embodiment for eliminating or attenuating the expression or function of the bipolar cell-regulating gene includes gene deletion of the bipolar cell-regulating gene. In this embodiment, a part or all of the nucleotide sequence of the bipolar cell-regulating gene is deleted. Examples of the deletion of a part of the nucleotide sequence include partial deletion of a region containing the transcription initiation codon or a region important for the function of the protein. It is possible to substitute a part or all of the nucleotide sequence of the bipolar cell-regulating gene, for example, with a drug-resistant gene or a fluorescence protein, for selecting a pluripotent stem cell having gene deletion.

Those skilled in the art can easily confirm that a gene is modified. One embodiment includes Southern blotting, PCR (Polymerase Chain Reaction), and sequencing.

Those skilled in the art can select genetically modified pluripotent stem cells. One embodiment includes a method involving introducing a drug resistance gene such as the neomycin resistance gene or a gene encoding fluorescence proteins such as GFP in genetic modification and selecting a genetically modified cell with a drug or the fluorescence of GFP.

The genetically modified pluripotent stem cell obtained in step (1) may be subjected to maintenance culture, expansion, storage, or another treatment before shifting to step (2), as long as its viable state and differentiation pluripotency is maintained.

The maintenance culture, expansion, and storage may be conducted by a method well known to those skilled in the art, such as the method described above.

When using cryopreserved pluripotent stem cells with a modified bipolar cell-regulating gene, step (1) may be replaced with a step of thawing the frozen cells.

Those skilled in the art can thaw cryopreserved cells by a well known method and use the thawed cells in step (2). In one embodiment, the cryopreserved cells may be thawed and then subjected to maintenance culture or expansion.

### 3-2. Regarding Step (2)

Specific examples of the method for obtaining a cell population for transplant comprising retinal cells from the cell population obtained in step (1) will be described. Examples of the method for inducing differentiation in the aforementioned step (2) include, but are not limited to, methods disclosed in WO2011/055855, WO2012/173207, WO2013/077425, WO2015/025967, WO2016/063985, WO2016/063986, PLOS One. 2010 Jan. 20; 5 (1): e8763., Stem Cells. 2011 Aug. 29 (8): 1206-18, Proc Natl Acad Sci USA 2014 Jun. 10, 111 (23): 8518-23, Nat Commun. 2014 Jun. 10; 5:4047. The pluripotent stem cell in which the bipolar cell-regulating gene has been modified by a method known to those skilled in the art may be subjected to other methods for inducing differentiation into a cell population for transplant comprising retinal cells or retinal tissue.

In one embodiment of step (2), a cell aggregate containing retinal cells may be obtained by the following steps (A) to (C).
(A): culturing the cell population of genetically modified pluripotent stem cells obtained in step (1) in suspension in a serum-free culture medium containing a Wnt signaling pathway inhibitor to form a cell aggregate of pluripotent stem cells,
(B): culturing the cell aggregate obtained in step (A) in suspension in a serum-free culture medium containing a basement membrane preparation,
(C): culturing the cell aggregate obtained in step (B) in suspension in a serum culture medium.

This method is disclosed, for example, in WO2013/077425, and WO2013/077425 may be referred to for greater detail.

The “serum-free culture medium” in the present invention means a culture medium not containing unadjusted or unpurified serum. In the present invention, a culture medium containing a purified ingredient derived from blood or derived from animal tissue (for example, a growth factor) is also included in the serum-free culture medium unless the culture medium contains unadjusted or unpurified serum.

As such a serum-free culture medium, a serum-free culture medium containing a proper amount (for example, about 0.5% to about 30%, preferably about 1% to about 20% in volume percentage) of commercially available KSR (manufactured by Life Technologies, trade name) may be used.

The Wnt signaling pathway inhibitor is not particularly limited, as long as it can suppress the signal transduction mediated by Wnt. For example, CKI-7 (N-(2-aminoethyl)-5-chloro-isoquinoline-8-sulfonamide), IWR-1-endo (IWR1e) are known Wnt signaling pathway inhibitors and commercially available as appropriate. Preferably, IWR1e is used as a Wnt signaling pathway inhibitor.

The concentration of the Wnt signaling pathway inhibitor to be used may be a concentration at which an aggregate of pluripotent stem cells is formed. For example, IWR1e is added to a culture medium at a concentration of about 0.1 μM to 100 μM, preferably about 1 μM to 10 μM, more preferably about 3 μM.

The Wnt signal pathway inhibitor may be added to a serum-free culture medium before starting the suspension culture. Moreover, it may be added to a serum-free culture medium within several days (for example, within 5 days) after starting the suspension culture. Preferably, the Wnt signal pathway inhibitor is added to a serum-free culture medium within 5 days, more preferably within 3 days after starting the suspension culture, and most preferably at the same time as starting the suspension culture. Moreover, the suspension culture is continued in the presence of the Wnt signal pathway inhibitor, preferably until the 18th day and more preferably until the 12th day after starting the suspension culture.

Examples of the experimental operation for forming the aggregate include a method involving trapping cells in a small space using a plate with small wells (96-well plate), micropores, or the like, a method involving aggregating cells by centrifugation for a short time using a small centrifuging tube, and the like.

When forming cell aggregates, the number of the pluripotent stem cells is not particularly limited as long as it allows formation of homogeneous aggregates of stem cells. For example, when using 96-well microwell plates, a liquid prepared at about 1×10³ to about 5×10⁴ cells, preferably about 3×10³ to about 3×10⁴ cells, more preferably about 5×10³ to about 2×10⁴ cells, most preferably around 9×10³ cells per well is added and the plate is left stand to allow the formation of cell aggregates.

The formation of aggregates of pluripotent stem cells can be determined by a person skilled in the art based on the size and the number of cells of the cell aggregates, the macroscopic morphology, the microscopic morphology and uniformity detected by the tissue staining analysis, the expression of differentiation and undifferentiation marker and the uniformity thereof, the regulation of expression of a differentiation marker and the synchronism, the reproducibility of differentiation efficiency between aggregates, and the like.

The basement membrane preparation refers to a preparation containing a basement membrane component having the function of controlling an epithelial cell-like cellular morphology, differentiation, proliferation, mobility, functional expression, and/or the like, when desired cells having basement membrane-forming ability are seeded and cultured thereon. Here, the “basement membrane component” refers to an extracellular matrix molecule in the form of thin film present between the epithelium cell layer and the stroma cell layer in animal tissue. The basement membrane preparation may be prepared, for example, by removing the cells having basement membrane-forming ability adhering on a support via a basement membrane using a solution that can lyse lipids of the cell or an alkaline solution. Examples of a preferred basement membrane preparation include products commercially available as basement membrane components (for example, Matrigel) or a preparation containing an extracellular matrix molecule known as a basement membrane component (for example, laminin, type IV collagen, heparan sulfate proteoglycan, entactin, or the like).

Examples of the amount of the basement membrane preparation to be used include preferably an amount of 1/20 to 1/200, more preferably an amount of around 1/100, based on the volume of culture medium, when Matigel is used. The basement membrane preparation may have been already added to a culture medium at the time of starting the culture of stem cells or be added to a serum-free culture medium, preferably within 5 days after starting the suspension culture and more preferably within 2 days after starting the suspension culture.

Examples of the serum that may be used include mammalian sera such as bovine serum, calf serum, fetal bovine serum, horse serum, foal serum, fetal horse serum, rabbit serum, leveret serum, fetal rabbit serum, and human serum.

The addition of the serum is conducted on the 7th day or later, more preferably on the 9th day or later, and most preferably on the 12th day after starting the suspension culture. The serum is added so that the concentration will be about 1 to 30%, preferably about 3 to 20%, more preferably around 10% (for example, 5% to 15%), in volume percentage.

In another embodiment of step (2), it is possible to obtain a cell aggregate containing retinal cells by a method comprising the following steps (D), (E) and (F).
(D): culturing the pluripotent stem cells with a modified bipolar cell-regulating gene obtained in step (1) in the absence of feeder cells in a culture medium containing 1) a TGFβ family signaling pathway inhibitor and/or a Sonic Hedgehog signaling pathway agent, and 2) an undifferentiated state-maintaining factor,
(E): culturing the pluripotent stem cells with a modified bipolar cell-regulating gene cultured in step (D) in a serum-free culture medium in suspension to form a cell aggregate,
(F): further culturing the cell aggregate obtained in step (E) in suspension in a culture medium containing a BMP signaling pathway agent.

This method is disclosed, for example, in WO2016/063985, and WO2016/063985 may be referred to for greater detail.

The TGFβ family signaling pathway inhibitor refers to a substance that inhibits the TGFβ family signaling pathway, that is, a signaling pathway transmitted by the Smad family, and specific examples thereof include TGFB signaling pathway inhibitors (for example, SB431542, LY-364947, SB-505124, A-83-01), Nodal/Activin signaling pathway inhibitors (for example, SB431542, A-83-01), and BMP signaling pathway inhibitors (for example, LDN193189, Dorsomorphin). These substances are marketed and available.

The Sonic Hedgehog (hereinafter, referred to as the “Shh”.) signaling pathway agent is a substance that can enhance the signaling transmitted by Shh. Examples of the Shh signaling pathway agent include PMA (Purmorphamine), SAG (Smoothened Agonist), and the like.

The concentration of the TGFβ family signaling pathway inhibitor and the Sonic Hedgehog signaling pathway agent may be a concentration that can induce the differentiation into retinal cells. For example, SB431542 is usually used at a concentration of 0.1 to 200 μM and preferably 2 to 50 μM. A-83-01 is usually used at a concentration of 0.05 to 50 μM and preferably 0.5 to 5 μM. LDN193189 is usually used at a concentration of 1 to 2000 nM and preferably 10 to 300 nM. SAG is usually used at a concentration of 1 to 2000 nM and preferably 10 to 700 nM. PMA is usually used at a concentration of 0.002 to 20 μM and preferably 0.02 to 2 μM.

In culturing the pluripotent stem cell under feeder-free conditions in step (D), the feeder-free culture medium containing an undifferentiated state-maintaining factor may be used as a culture medium.

In culturing the pluripotent stem cell under feeder-free conditions in step (D), an appropriate matrix may be used as a scaffold to provide a scaffold for pluripotent stem cells in the place of feeder cells. Examples of matrices that may be used as a scaffold include Laminin (Nat Biotechnol 28, 611-615, (2010)), Laminin fragments (Nat Commun 3, 1236, (2012)), a basement membrane preparation (Nat Biotechnol 19, 971-974, (2001)), gelatin, collagen, heparan sulfate proteoglycan, entactin, vitronectin, and the like.

The culture time of pluripotent stem cells in step (D) is not particularly limited, as long as it is within the range that allows improvement of the quality of cell aggregates formed in step (E), but it is usually 0.5 to 144 hours. In one embodiment, the culture time is preferably 2 to 96 hours, more preferably 6 to 48 hours, further preferably 12 to 48 hours, and even more preferably 18 to 28 hours (for example, 24 hours).

The preparation of the serum-free culture medium and the formation of the cell aggregate may be performed as described above.

In one embodiment, the culture medium used in step (E) comprises a Sonic Hedgehog signaling pathway agent. As the Sonic Hedgehog signaling pathway agent, those described above may be used at a concentration described above. The Sonic Hedgehog signaling pathway agent is preferably contained in the culture medium from the start of the suspension culture. A ROCK inhibitor (for example, Y-27632) may be added to the culture medium. The culture time is, for example, 12 hours to 6 days.

The BMP signaling pathway agent is a substance that can enhance a signaling pathway transmitted by BMP. Examples of the BMP signaling pathway agent include a BMP protein such as BMP2, BMP4, or BMP7, a GDF protein such as GDF7, an anti-BMP receptor antibody, or a partial BMP peptide. The BMP2 protein, the BMP4 protein, and the BMP7 protein are available, for example, from R&D Systems, Inc. and the GDF7 protein is available, for example, from Wako Pure Chemical Industries, Ltd.

Examples of the culture medium to be used include serum-free culture media or serum culture media (preferably serum-free culture media) in which the BMP signaling pathway agent is added. The serum-free culture media and the serum culture media may be prepared as described above.

The concentration of the BMP signaling pathway agent may be a concentration that can induce the differentiation into retinal cells. For example, the human BMP4 protein is added to a culture medium at a concentration of about 0.01 nM to about 1 μM, preferably about 0.1 nM to about 100 nM, more preferably about 1 nM to about 10 nM, and more preferably about 1.5 nM (55 ng/ml).

The BMP signaling pathway agent may be added to a culture medium about 24 hours or more after starting the suspension culture in step (D), and for example, it may be added to a culture medium within several days (for example, within 15 days) after starting the suspension culture. Preferably, the BMP signaling pathway agent may be added to a culture medium between the 1st day and the 15th day, more preferably between the 1st day and the 9th day, most preferably the 3rd day after starting the suspension culture.

The culture conditions such as the culture temperature and the CO₂ concentration in step (A) to step (F) may be set as appropriate.

The culture temperature is, for example, about 30° C. to about 40° C. and preferably about 37° C. Moreover, the CO₂ concentration is, for example, about 1% to about 10% and preferably about 5%.

It is possible to produce retinal cells at various differentiation stages by changing the culture period in step (C) or step (F) described above. Accordingly, it is possible to produce retinal cells comprising immature retinal cells (for example, retinal progenitor cells or photoreceptor progenitor cells) and mature retinal cells (for example, photoreceptor cells) at various ratios. It is possible to increase the ratio of mature retinal cells by extending the culture period of step (C) or step (F).

It is also possible to produce a ciliary marginal zone-like structure by culturing the cell aggregate of retinal cells obtained in the method described above in the presence or absence of the BMP signaling pathway agent in a serum-free culture medium or a serum culture medium containing the Wnt signaling pathway agent and/or the FGF signaling pathway inhibitor for a time period of from about 3 days to 6 days and then in a serum-free culture medium or a serum culture medium containing neither Wnt signaling pathway agent nor FGF signaling pathway inhibitor for from about 30 days to 60 days.

The ciliary marginal zone-like structure is a structure similar to the ciliary marginal zone. Examples of the “ciliary marginal zone (CMZ)” include a region of a retina that is a tissue present in the border region between the retinal tissue (specifically the neural retina) and the retinal pigment epithelium in the living retina and that contains tissue stem cells (retinal stem cells). The ciliary marginal zone is also called as the ciliary margin or the retinal margin. It is known that the ciliary marginal zone plays an important role in the supply of retinal progenitor cells or differentiated cells to retinal tissue and the maintenance of retinal tissue structure. Examples of marker genes for the ciliary marginal zone include the Rdh10 gene (positive), the Otx1 gene (positive), and the Zic1 (positive).

The Wnt signaling pathway agent is not particularly limited, as long as it can enhance the signal transduction transmitted by Wnt. Specific examples of the Wnt signaling pathway agent include a GSK3β inhibitor (for example, 6-Bromoindirubin-3′-oxime (BIO), CHIR99021, Kenpaullone). For example, CHIR99021 is added at a concentration of about 0.1 μM to about 100 μM and preferably about 1 μM to about 30 μM.

The FGF signaling pathway inhibitor is not particularly limited, as long as it can inhibit the signal transduction transmitted by FGF. Examples of the FGF signaling pathway inhibitor include SU-5402, AZD4547, BGJ398, and the like. For example, SU-5402 is added at a concentration of about 0.1 μM to about 100 μM, preferably about 1 μM to about 30 μM, and more preferably about 5 μM.

The retinal cells with a modified bipolar cell-regulating gene may be produced by a method described above, but it is not limited to these methods.

In the cell population for transplant comprising the retinal cell obtained in step (2), the sum of the number of retinal progenitor cells, photoreceptor progenitor cells, and photoreceptor cells is 10% or more (preferably 20% or more, 30% or more, 40% or more, 50% or more, 60% or more, 70% or more, 80% or more, or 90% or more) of the total number of cells. Moreover, when continuing the culture of the cell population for transplant comprising the retinal cell obtained in step (2) (for example, after 120 days from the start of step (2)), or when transplanting the cell population for transplant comprising the retinal cell obtained in step (2) into the living body, the neural retinal layer is formed in a retina in vitro or in a transplanted recipient retina and cells that constitute the neural retinal layer increase. At this stage, the sum of the number of retinal progenitor cells, photoreceptor progenitor cells, and photoreceptor cells may be 30% or more (preferably 40% or more or 50% or more) of the total number of cells, and the sum of the numbers of bipolar cells progenitor cells, cone bipolar cells, and rod bipolar cells may be 30% or less (preferably 20% or less, 10% or less, or 5% or less) of the total number of cells. Moreover, the ratio of the number of bipolar cells to the number of amacrine cells decreases (for example, 70% or less, 60% or less, or 50% or less).

In one embodiment of the present invention, the retinal progenitor cells are Chx10-positive cells, the photoreceptor progenitor cells are Crx-positive cells, and the photoreceptor cells are recoverin-positive cells.

Those skilled in the art can measure the ratio of retinal progenitor cells, photoreceptor progenitor cells, and photoreceptor cells in the cell population obtained in step (2) by a well known method. In one embodiment, it is possible to measure the ratio of Chx10-positive cells, Crx-positive cells, recoverin-positive cells contained by a method such as immunostaining or flow cytometry.

Those skilled in the art can confirm that the modification of the bipolar cell-regulating gene is maintained by a well known method, for example, the method described above.

The undifferentiation into bipolar cells or degenerative death of bipolar cells may be confirmed, for example, based on the presence or absence of the expression of the bipolar cell marker PKCα, Chx10, or the like determined by the method such as immunostaining or flow cytometry.

The dysfunction of bipolar cells may be confirmed by a method such as immunohistological analysis or electrophysiological analysis. For example, if the synaptic connection with photoreceptor cells or neuronal firing is not observed, it is possible to determine that bipolar cells are dysfunctional.

Moreover, it is possible to confirm that normal photoreceptor cells are induced, for example, based on the presence or absence of the expression of the photoreceptor cell marker recoverin or the like determined by a method such as immunostaining or flow cytometry.

The presence or absence of the aforementioned cells and the ratio thereof may also be confirmed from tissue after the transplant.

### 4. Regarding Transplant of Cell Population for Transplant Comprising Retinal Cell

The cell population for transplant comprising a retinal cell produced by the method described above may be transplanted into a subject (for example, a mammal) in need of transplant and it is possible to improve the visual function of the subject by the transplant. Examples of a mammalian animal that may be a subject include a human, a mouse, a rat, a guinea pig, a hamster, a rabbit, a cat, a dog, a sheep, a pig, a cow, a horse, a goat, a monkey, and the like.

The cell population for transplant comprising retinal cells may be transplanted in the form of a cell aggregate. Moreover, it is possible to cut the retinal tissue that is obtained in the method of production according to the present invention into appropriate size using tweezers, a knife, scissors, or the like to make a sheet agent (a cell sheet) and then transplant the agent. Examples thereof include transplanting one or a plurality of pieces of the cell sheet (for example, 300 μm in diameter, 50 μm in height) cut out from one cell aggregate depending on the area of the region in which photoreceptor cells are degenerated. Those skilled in the art can select the number of pieces of the cell sheet depending on the area of the region of degenerative death. Furthermore, it is possible to subject the produced retinal cells to an operation such as an enzymatic treatment or pipetting and transplant the cells in a form of cell suspension.

The cell population for transplant comprising retinal cells is transplanted preferably after being made into a pharmaceutical composition.

The transplant of the cell suspension is carried out, for example, by a method involving transplanting the cell suspension under retina using a needle. The transplantation of the cell sheet is carried out, for example, by incising a part of the eyeball and transplanting the cell sheet to the damaged site or lesion site through the incision.

After transplantation, at least a part of taken immature retinal cells is induced to differentiate into mature retinal cells in the in vivo (intraocular) environment of the subject. Here, the “photoreceptor cells induced after transplantation” means photoreceptor cells induced and differentiated from taken retinal progenitor cells or photoreceptor progenitor cells after transplantation in the eye of the subject.

Here, the “take” in the present invention means that the transplanted cells survive in the living body for a long period of time (for example, 30 days or more, 60 days or more, or 90 days or more) and are adhered to and remain in the organ.

The “functional take” in the present invention means a state in which the transplanted cells are taken and perform their original function in the body.

The original function of the photoreceptor cells is to come in contact with bipolar cells to form the synapse in the body and to transmit electrical signals converted in the photoreceptor cells to bipolar cells. Therefore, the “functional take of photoreceptor cells” in the present invention means that transplanted photoreceptor cells (including photoreceptor cells induced after transplantation) is taken in a state in which the photoreceptor cells come in contact with host bipolar cells to form the synapse and electrical signals converted in the photoreceptor cells are transmitted to bipolar cells through the synapse. It is possible to confirm that photoreceptor cells and bipolar cells have formed the synapse by staining a photoreceptor cell synapse marker (for example, Ctbp2, Basoon, Cacnalf, ELFN1) or a bipolar cell synapse marker (for example, mGluR6, Cacnals, TRPM1). Specifically, it is possible to confirm the synapse formation by staining the aforementioned synapse marker on the contact surface of recoverin-positive cells (photoreceptor cells) and PKCα-positive cells (bipolar cells) by a technique such as immunostaining. In the present invention, the state in which the photoreceptor cell layer in the transplant come in contact with the retinal layer containing host bipolar cells may be considered as the functional take of photoreceptor cells (Stem Cells, 31, 1149-1159, (2013)).

The “contact” in the present invention refers to the state in which cells are physically in close proximity and the synaptic connection is suggested.

The “ratio of contact” in the present invention refers to the ratio of length of contact between the photoreceptor cell layer in the transplanted retinal tissue and the retinal layer containing host bipolar cells to the length of a longer side of the transplanted retinal tissue.

The “functional take rate” in the present invention means the ratio of the cells that have achieved functional take to the cells transplanted. The functional take rate of transplanted photoreceptor cells may be determined from the aforementioned contact ratio.

The functional take rate of transplanted photoreceptor cells (including photoreceptor cells induced after transplantation) by transplanting the cell population for transplant comprising retinal cells produced by the method according to the present invention is 10% or more, preferably 20% or more, more preferably 40% or more, further preferably 50% or more, and particularly preferably 60% or more.

The higher the take rate and/or the functional take rate of transplanted photoreceptor cells (including photoreceptor cells induced after transplantation) is, the greater the improvement of the visual function after the transplantation.

Accordingly, the present invention provides a method for improving the take rate and/or the functional take rate of transplanted photoreceptor cells (including photoreceptor cells induced after transplantation).

### 5. Pharmaceutical Composition

The present invention provides a pharmaceutical composition comprising an effective amount of a cell population for transplant comprising retinal cells produced by the aforementioned method of production. The effective amount of the cell population for transplant varies depending on the purpose of administration, the mode of administration, and the state (sex, age, body weight, conditions) of the subject of administration, but may be, for example, 1×10⁵ to 1×10⁷ cells (for example, 1×10⁵ cells, 1×10⁶ cells, or 1×10⁷ cells) in the number of cells.

The pharmaceutical composition comprises an effective amount of the aforementioned cell population for transplant produced by the method of production according to the present invention and a pharmaceutically acceptable carrier.

As the pharmaceutically acceptable carrier, a physiological aqueous solvent (physiological saline, buffer solution, a serum-free culture medium, or the like) may be used. A preservative, a stabilizer, a reducing agent, an isotonizing agent, or the like that is usually used in medicaments in transplant medicine containing tissue or cells to be transplanted may be added to the pharmaceutical composition as needed.

The pharmaceutical composition according to the present invention may be produced as a cell suspension by suspending the aforementioned cell population for transplant produced by the aforementioned method of production with an appropriate physiological aqueous solvent. If needed, it is also possible to add a cryoprotective agent to the aforementioned cell population for transplant to cryopreserve the cell population, thaw the cryopreserved cell population before using, wash the thawed cell population with a buffer solution, and use it for transplant medicine.

The retinal tissue comprising the cell population for transplant that is obtained by the method of production according to the present invention may be cut into appropriate size using tweezers, a knife, scissors, or the like to make it a sheet agent.

Moreover, the cell population for transplant that is obtained by the method of production according to the present invention may also be formed into a sheet to make it a sheet agent that is a cell sheet, by conducting the adherent culture in step (2) of inducing differentiation.

The pharmaceutical composition according to the present invention is useful as a therapeutic agent for diseases based on disorders of retinal cells.

### 6. Therapeutic Agent and Method of Treatment

The cell population for transplant comprising retinal cells produced by the method of production according to the present invention is useful in transplant medicine for diseases based on disorders of retinal tissue (including disorders of retinal progenitor cells or retinal cells). Accordingly, the present invention provides a therapeutic agent for a disease based on a disorder of retinal tissue, comprising a cell population for transplant comprising retinal cells produced by the method of production according to the present invention. Moreover, the present invention provides a method of treatment, comprising administering (transplanting) the therapeutic agent in the form of a suspension or a sheet agent to a patient. The cell population for transplant comprising retinal cells produced by the method of production according to the present invention may be used as a therapeutic agent for a disease based on a disorder of retinal tissue or for supplementing the damaged site in a condition of retinal tissue damage. It is possible to treat diseases based on a disorder of retinal tissue or a condition of retinal tissue damage by transplanting a cell population for transplant comprising retinal cells produced by the method of production according to the present invention to a patient in need of transplantation with a disease based on a disorder of retinal tissue or a condition of retinal tissue damage and supplementing the retinal cells or damaged retinal tissue itself. Examples of the disease based on a disorder of retinal tissue include eye diseases such as macular degeneration, age-related macular degeneration, retinitis pigmentosa, glaucoma, corneal disease, retinal detachment, central serous chorioretinopathy, cone dystrophy, cone-rod dystrophy, and the like. Examples of the condition of retinal tissue damage include the condition in which photoreceptor cells are dead in degeneration, and the like.

While the rejection due to the difference in histocompatible antigens often becomes the problem in transplant medicine, it is possible to overcome the problem by using pluripotent stem cells (for example, induced pluripotent stem cells) established from somatic cells of the recipient of the transplantation. Accordingly, in a preferred embodiment of the present invention, immunological self retinal tissue or retinal cells for the recipient are produced by using pluripotent stem cells (for example, induced pluripotent stem cells) established from somatic cells of the recipient as pluripotent stem cells, and are transplanted into the recipient.

Moreover, allo (nonself) retinal tissue or retinal cells may be prepared from pluripotent stem cells (for example, induced pluripotent stem cells) established from somatic cells of a nonself who is immunologically compatible (for example, having a compatible HLA or MHC type) with the recipient and transplanted to the recipient.

### EXAMPLES

Hereinafter, the present invention is described in detail in reference with examples but is not limited thereto.

### Example 1: Example of Transplanting the Retinal Tissue Derived from Mouse iPS Cells Wherein the Function of a Bhlhb4 Gene is Deleted to Reduce Rod Bipolar Cells in the Transplanted Tissue

Mouse iPS cells (Stem Cells, 31, 1149-1159, (2013), Proc. Natl. Acad. Sci. USA, 103, pp. 3890-3895, (2006)) having an Nrl::eGFP reporter established from an Nrl::eGFP mouse were cultured while maintaining undifferentiated state in accordance with the method described in Nature biotechnology, 26 (2), 215-24, (2008).

A CRISPR/Cas9 system was used as a method for causing loss of function of a Bhlhb4 gene (mouse ortholog of BHLHE23 gene). The target sequence sites can be cleaved by intracellular expression of SpCas9 and target sequences sgRNA (SEQ ID NO 16: ccgagctcaagtcgctgtcg, SEQ ID NO: 17 cgcgccttggtgagaaggcg). A plasmid into which SpCas9 and the target sequences were incorporated was introduced to mouse iPS cells by electroporation (product name: Nucreofector, manufactured by Lonza). CRISPR/Cas9, gRNA designed so as to delete a region comprising an initiation codon of the Bhlhb4 gene, and a plasmid into which a puromycin-resistance gene was incorporated were introduced to the mouse iPS cells. The introduced cells were selected using puromycin and colonies were picked up, whereby a Bhlhb4 gene-deleted line was established. The deletion at the sites of interest in the Bhlhb4 gene was confirmed by PCR and agarose gel electrophoresis, or by reading the nucleotide sequence information.

SFEBq method described in Non Patent Literature 5, which is the modified method described in Non Patent Literature 2, was used as the method for inducing differentiation into the retina. The induction of differentiation was operated as follows. Mouse iPS cells were enzymatically treated and dispersed into single cells, and subsequently suspension cultured in a 96-well plate at 3000 cells/well. From day 0 to day 1 after the initiation of inducing differentiation, the above cells were cultured in a culture medium to which AGN193109 (0.1 μM, manufactured by Toronto Research Chemicals) was added, and subsequently up to day 8 the above cells were cultured in a culture medium to which AGN193109 and Growth Factor Reduced Matrigel (2% v/v, manufactured by BD Biosciences) were added. Note that the “day N after the initiation of inducing differentiation” refers to a period from N days after (N x 24 hours after) the initiation of inducing differentiation to immediately before N+1 day ((N+1)×24 hours) have passed (the same applies hereinafter). For the culture medium, G-MEM (5% KSR, 0.1 mM non-essential amino acid, 1 mM pyruvate, 0.1 mM 2-mercaptoethanol) was used. On day 8 after the initiation of inducing differentiation, an optic vesicle wherein the epithelial structure of retinal progenitor tissue is projected like a pouch was cut off from the cell aggregate using a No. 11 Blade and suspension cultured in a culture medium of DMEM/F12 (N2 Supplement, 10% FBS, 0.5 μM all-trans-retinoic acid, 1 mM L-taurine) under environment of 40% O₂ and 5% CO₂. Three-dimensional retina was cut off on day 15 after the initiation of inducing differentiation and subretinally transplanted using a syringe into a rd1 mouse, a photoreceptor cell degeneration model. On the age equivalent to 30- to 50-day differentiation after transplant, the ocular tissue was fixed in paraformaldehyde (PFA fixation) and replaced with sucrose. Tissue sections were prepared using a cryostat. Rod bipolar cells, amacrine cells, and horizontal cells in the tissue section were stained by immunostaining using respectively an anti-PKCα antibody (product name: Rabbit Anti-Protein Kinase Co antibody, manufactured by Sigma), an anti-calretinin antibody (product name: Rabbit Anti-Calretinin Antibody, manufactured by Millipore), and an anti-calbindin antibody (product name: Monoclonal Anti Calbindin-D-28K antibody, manufactured by Sigma) and the grafts after transplant were evaluated.

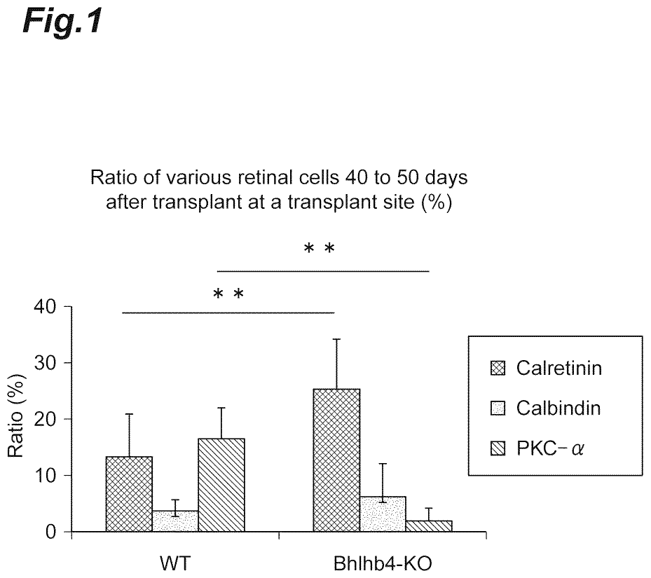

Fluorescence observation was performed on the stained tissue using a confocal microscope (product name: TCS SP8, manufactured by Leica) to investigate a ratio of each cell in the graft. As a result, the ratio of rod bipolar cells was found to have been significantly reduced when the retinal tissue wherein the Bhlhb4 gene was deleted was transplanted compared with the case where the retinal tissue wherein the Bhlhb4 gene was not deleted was transplanted (FIG. 1).

### Example 2: Example of Transplanting the Retinal Tissue Derived from Mouse iPS Cells Wherein the Function of a Bhlhb4 Gene is Deleted to Enhance a Contact Ratio with Host Rod Bipolar Cells

Mouse iPS cells having an Nrl::eGFP reporter established from an Nrl::eGFP mouse were cultured while maintaining undifferentiated state in accordance with the method described in Nature biotechnology, 26(2), 215-24, (2008).

The method for inducing differentiation into the retina and the transplant method used were the same methods as in Example 1. On the age equivalent to about 40- to 90-day differentiation after transplant, the ocular tissue was fixed in PFA and replaced with sucrose. Tissue sections were prepared using a cryostat. Rod bipolar cells in the tissue section were stained by immunostaining using an anti-PKCo antibody (product name: Rabbit Anti-Protein Kinase Co antibody, manufactured by Sigma).

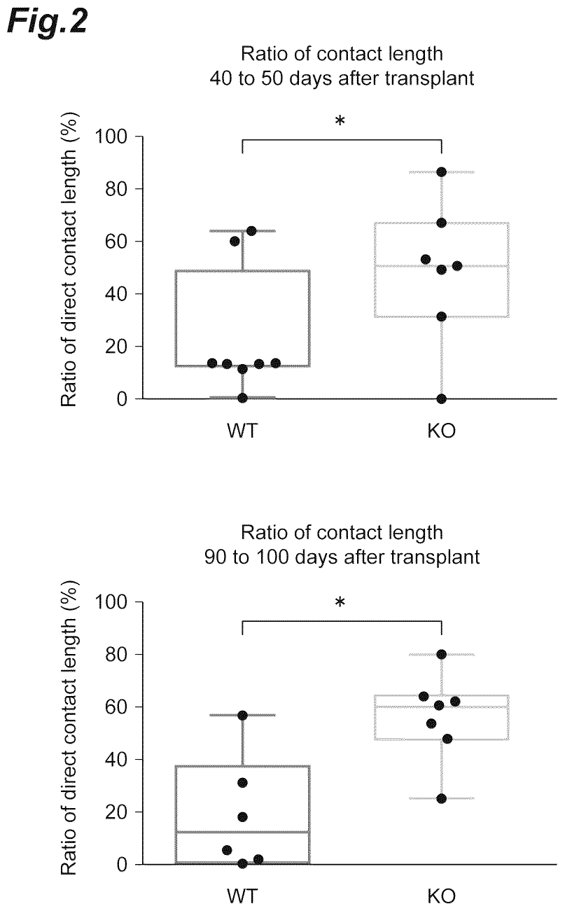

Fluorescence observation was performed on the stained tissue using a confocal microscope (product name: TCS SP8, manufactured by Leica) to investigate a ratio of the length of the part along which the host rod bipolar cells and the eGFP-positive transplanted photoreceptor cells were in contact with each other to the length of the entire part wherein the graft was present. As a result, the ratio of the photoreceptor cells derived from the graft in contact with the host rod bipolar cells was found to have been significantly increased when the retinal tissue wherein the Bhlhb4 gene was deleted was transplanted compared with the case where the retinal tissue wherein the Bhlhb4 gene was not deleted was transplanted (FIG. 2).

### Example 3: Example of Transplanting the Retinal Tissue Derived from Mouse iPS Cells Wherein the Function of an ISL1 Gene is Deleted to Reduce Rod Bipolar Cells, Amacrine Cells, and Horizontal Cells in the Transplanted Tissue

Mouse iPS cells having an Nrl::eGFP reporter established from an Nrl::eGFP mouse were cultured while maintaining undifferentiated state in accordance with the method described in Nature biotechnology, 26(2), 215-24, (2008).

A CRISPR/Cas9 system was used as a method for deleting the function of an ISL1 gene. CRISPR/Cas9, target sequences designed so as to delete the first and second exons of the ILS1 gene (SEQ ID NO 18: tcttcaatagcacgcgggaa, SEQ ID NO 19: tcctaagccataaagcgctt), and a plasmid into which a puromycin-resistance gene was incorporated were introduced to the mouse iPS cells by electroporation (Nucreofector, manufactured by Lonza). The introduced cells were selected using puromycin and colonies were picked up, whereby an ISL1 gene-deleted line was established. The deletion at the sites of interest in the ISL1 gene was confirmed by PCR and agarose gel electrophoresis, or by reading the nucleotide sequence information.

SFEBq method described in Non Patent Literature 5, which is the modified method described in Non Patent Literature 2, was used as the method for inducing differentiation into the retina. The induction of differentiation was operated as follows. Mouse iPS cells were enzymatically treated and dispersed into single cells, and subsequently suspension cultured in a 96-well plate at 3000 cells/well. From day 0 to day 1 after the initiation of inducing differentiation, the above cells were cultured in a culture medium to which AGN193109 (0.1 μM) was added, and subsequently up to day 8 the above cells were cultured in a culture medium to which AGN193109 (0.1 μM) and Growth Factor Reduced Matrigel (2% v/v) were added. For the culture medium, G-MEM (5% KSR, 0.1 mM non-essential amino acid, 1 mM pyruvate, 0.1 mM 2-mercaptoethanol) was used. On day 8 after the initiation of inducing differentiation, an optic vesicle was cut off using a No. 11 Blade and suspension cultured in a culture medium of DMEM/F12 (N2 Supplement, 10% FBS, 0.5 μM all-trans-retinoic acid, 1 mM L-taurine) under environment of 40% O₂ and 5% CO₂. Three-dimensional retina was cut off on day 15 after the initiation of inducing differentiation and subretinally transplanted using a syringe into a rdl mouse, a photoreceptor cell degeneration model. On days 30 to 50 after transplant, the ocular tissue was fixed in PFA and replaced with sucrose.

Sections were prepared using a cryostat. Rod bipolar cells, amacrine cells, and horizontal cells were stained by immunostaining using respectively an anti-PKCo antibody, an anti-calretinin antibody, and an anti-calbindin antibody and the grafts after transplant were evaluated.

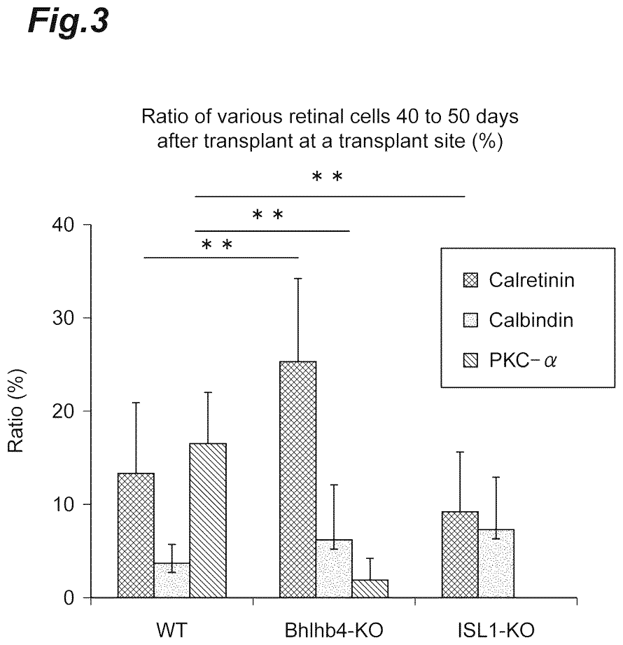

Fluorescence observation was performed on the stained tissue using a confocal microscope (manufactured by Leica) to investigate a ratio of each cell in the graft. As a result, the ratio of rod bipolar cells was found to have been significantly reduced when the retinal tissue wherein the ISL1 gene was deleted was transplanted compared with the case where the retinal tissue wherein the ISL1 gene was not deleted was transplanted (FIG. 3).

### Example 4: Example of Transplanting the Retinal Tissue Derived from Mouse iPS Cells Wherein the Function of a Bipolar Cell-Regulating Gene is Deleted and Suggesting the Synaptic Connection Between the Host Bipolar Cells and the Transplanted Photoreceptor Cells in the Transplanted Tissue

Mouse iPS cells having an Nrl::eGFP reporter established from an Nrl::eGFP mouse were cultured while maintaining undifferentiated state in accordance with the method described in Nature biotechnology, 26 (2), 215-24, (2008).

The method for deleting the Bhlhb4 gene or the ISL1 gene used was the same method as in Examples 1 and 3.

SFEBq method described in Non Patent Literature 5, which is the modified method described in Non Patent Literature 2, was used as the method for inducing differentiation into the retina. The induction of differentiation was operated as follows. Mouse iPS cells were enzymatically treated and dispersed into single cells, and subsequently suspension cultured in a 96-well plate at 3000 cells/well. From day 0 to day 1 after the initiation of inducing differentiation, the above cells were cultured in a culture medium to which AGN193109 (0.1 μM) was added, and subsequently up to day 8 the above cells were cultured in a culture medium to which AGN193109 (0.1 μM) and Growth Factor Reduced Matrigel (2% v/v) were added. For the culture medium, G-MEM (5% KSR, 0.1 mM non-essential amino acid, 1 mM pyruvate, 0.1 mM 2-mercaptoethanol) was used. On day 8 after the initiation of inducing differentiation, an optic vesicle was cut off using a No. 11 Blade and suspension cultured in a culture medium of DMEM/F12 (N2 Supplement, 10% FBS, 0.5 μM all-trans-retinoic acid, 1 mM L-taurine) under environment of 40% O₂ and 5% CO₂. Three-dimensional retina was cut off on day 15 after the initiation of inducing differentiation and subretinally transplanted using a syringe into a rdl mouse, a photoreceptor cell degeneration model. On days 30 to 50 after transplant, the ocular tissue was fixed in PFA and replaced with sucrose. Sections were prepared using a cryostat. Synaptic terminals of the rod bipolar cells and the photoreceptor cells in the section were stained by immunostaining using respectively an anti-PKCα antibody and an anti-Ctbp2 antibody and the grafts after transplant were evaluated.

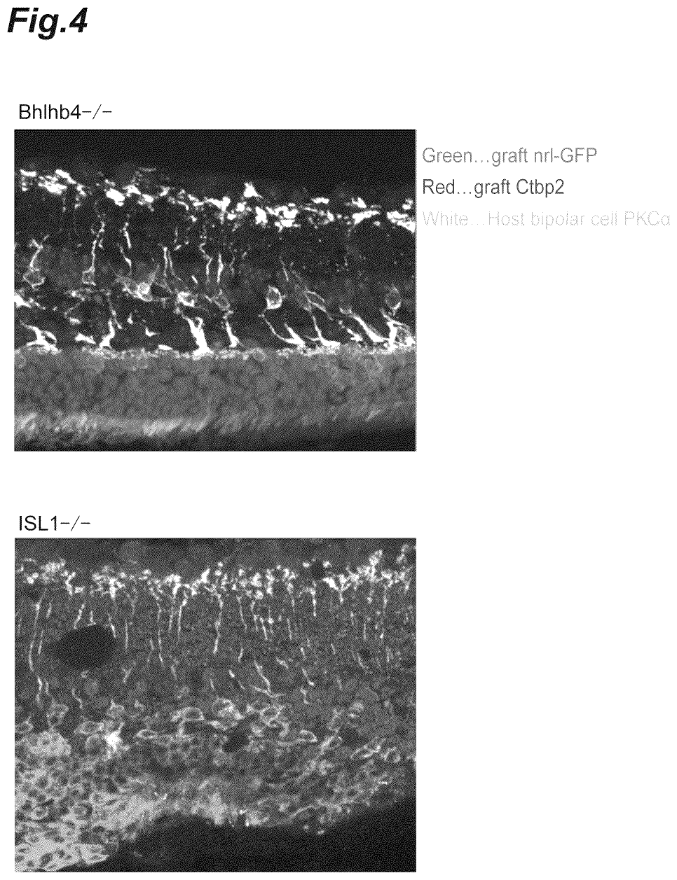

Fluorescence observation was performed on the stained tissue using a confocal microscope (manufactured by Leica) to evaluate the synaptic connection between the host bipolar cells and the transplanted photoreceptor cells in the transplanted tissue. As a result, the retinal tissue wherein the BhlhB4 and ISL1 genes were deleted, when transplanted, was suggested to have been synaptically connected with the host bipolar cells (FIG. 4).

### Example 5: Example of Evaluating the Reduction of Rod Bipolar Cells, Amacrine Cells, and Horizontal Cells Due to Loss of Function of a Bipolar Cell-Regulating Gene

Mouse iPS cells having an Nrl::eGFP reporter established from an Nrl::eGFP mouse were cultured while maintaining undifferentiated state in accordance with the method described in Nature biotechnology, 26(2), 215-24, (2008).

The method for deleting the Bhlhb4 gene or the ISL1 gene used was the same method as in Examples 1 and 3.

SFEBq method described in Non Patent Literature 5, which is the modified method described in Non Patent Literature 2, was used as the method for inducing differentiation into the retina. The induction of differentiation was operated as follows. Mouse iPS cells were enzymatically treated and dispersed into single cells, and subsequently suspension cultured in a 96-well plate at 3000 cells/well. From day 0 to day 1 after the initiation of inducing differentiation, the above cells were cultured in a culture medium to which AGN193109 (0.1 μM) was added, and subsequently up to day 8 the above cells were cultured in a culture medium to which AGN193109 (0.1 μM) and Growth Factor Reduced Matrigel (2% v/v) were added. For the culture medium, G-MEM (5% KSR, 0.1 mM non-essential amino acid, 1 mM pyruvate, 0.1 mM 2-mercaptoethanol) was used. On day 8 after the initiation of inducing differentiation, an optic vesicle was cut off using a No. 11 Blade and suspension cultured in a culture medium of DMEM/F12 (N2 Supplement, 10% FBS, 0.5 μM all-trans-retinoic acid, 1 mM L-taurine) under environment of 40% O₂ and 5% CO₂. On day 29 after the initiation of inducing differentiation, the tissue was fixed in PFA and replaced with sucrose to prepare a section. Mature photoreceptor cells using an anti-rhodopsin antibody (manufactured by Sigma), rod bipolar cells using an anti-PKCo antibody (manufactured by Sigma), amacrine cells, horizontal cells, and bipolar cells using an anti-ISL1 antibody (product name: Anti Islet 1 Antibody, manufactured by DSHB), photoreceptor cells using an anti-recoverin antibody (product name: Anti Recoverin Antibody, manufactured by Millipore), bipolar cells using an anti-Chx10 antibody (product name: Anti Chx10 Antibody, manufactured by Exalpha), and Muller glial cells using an anti-GS antibody (product name: Anti GS Antibody, manufactured by Millipore) and an anti-GFAP antibody (product name: Anti GFAP antibody, manufactured by dako) were stained by immunostaining, and fluorescence observation was performed using a confocal microscope (TCS, SP8, manufactured by Leica).

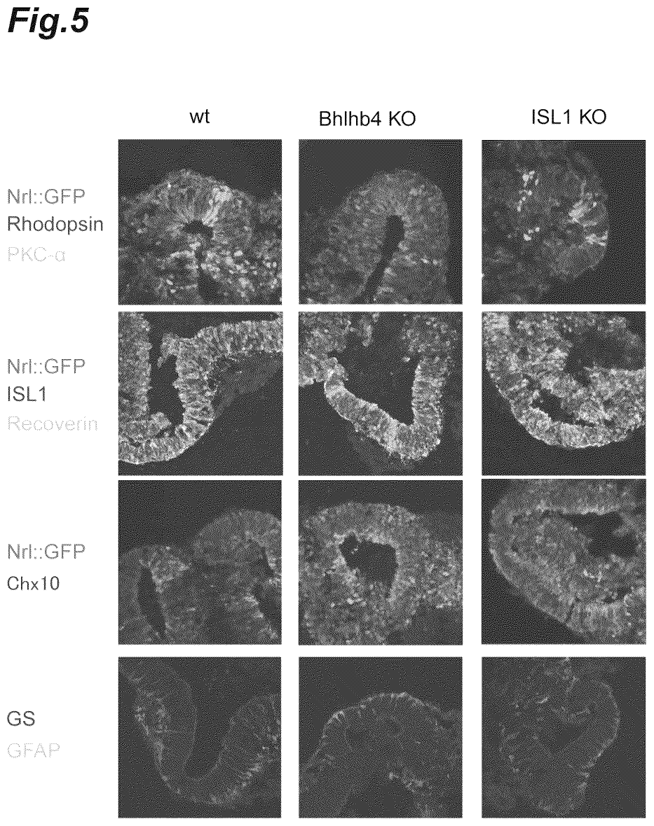

Fluorescence observation was performed on the stained tissue using a confocal microscope (manufactured by Leica) and a ratio of each cell in the differentiation-induced retinal tissue was investigated. As a result, the aggregate (aggregate cluster) derived from iPS cells wherein the Bhlhb4 gene or the ISL1 gene was deleted had Nrl-positive rod photoreceptor cells, rhodopsin-positive mature photoreceptor cells, recoverin-positive photoreceptor cells, and GS-positive and GFAP-positive Muller glial cells were observed as in the aggregate wherein the Bhlhb4 gene and the ISL1 gene were not deleted. On the other hand, PKCα-positive rod bipolar cells, Chx10-positive bipolar cells, and ISL1-positive cells were reduced (FIG. 5). Thus, when iPS cells wherein the BhlhB4 gene or the ISL1 gene was deleted were long-term cultured under conditions of differentiating into the retinal cells, bipolar cells and ISL1-positive cells were found to be significantly reduced without affecting photoreceptor cells and Muller glia cells (FIG. 5).

### Example 6: Example of Establishing a Human ES Cell Line Wherein the Function of an ISL1 Gene is Deleted

Human ES cells (Kh-ES1 line, Non Patent Literature 3) genetically modified so as to have a Crx::Venus reporter gene were cultured under feeder free conditions in accordance with the method described in “Scientific Reports, 4, 3594 (2014)”. StemFit medium (product name: AK03N, manufactured by Ajinomoto Co., Inc.) was used as the feeder free culture medium and Laminin511-E8 (product name, manufactured by Nippi Inc.) was used as a scaffold in place of the feeder cells.

Maintenance culture of the human ES cells was operated specifically as follows. Human ES cells (KhES-1 line) reached subconfluence (about 60% of a culture area is covered with cells) was washed with PBS and dispersed into single cells using TrypLE Select (product name, manufactured by Life Technologies). Subsequently, the human ES cells dispersed into single cells were inoculated in a plastic culture dish coated with Laminin511-E8 and cultured in the presence of Y27632 (ROCK inhibitor, 10 μM) under feeder free conditions in StemFit medium. When a 6-well plate (manufactured by IWAKI & CO., LTD., for cell culture, culture area 9.4 cm²) was used as the plastic culture dish, the inoculated number of the above human ES cells dispersed into single cells was 1.2×10⁴ cells/well. One day after inoculation, the medium was replaced with Y27632 free StemFit medium. Thereafter, the medium was replaced with Y27632 free StemFit medium once every 1 to 2 days. Subsequently, 6 days after inoculation, the cells were cultured until subconfluency was reached.

The following operation was performed as an introduction example of a CRISPR/Cas9 system in step (1) of a method for producing a cell population for transplant comprising retinal cells. Human ES cells which reached subconfluence were washed with PBS, dispersed into single cells using TrypLE Select, and collected. Subsequently, CRISPR/Cas9, gRNA designed so as to delete the first and second exons of the ISL1 gene (SEQ ID NO 20: CCAACTCCGCCGGCTTAAAT, SEQ ID NO 21: GGGAGGTTAATACTTCGGAG), and a plasmid into which a puromycin-resistance gene was incorporated were introduced to the human ES cells using electroporation (product name: Nucreofector, manufactured by Lonza).

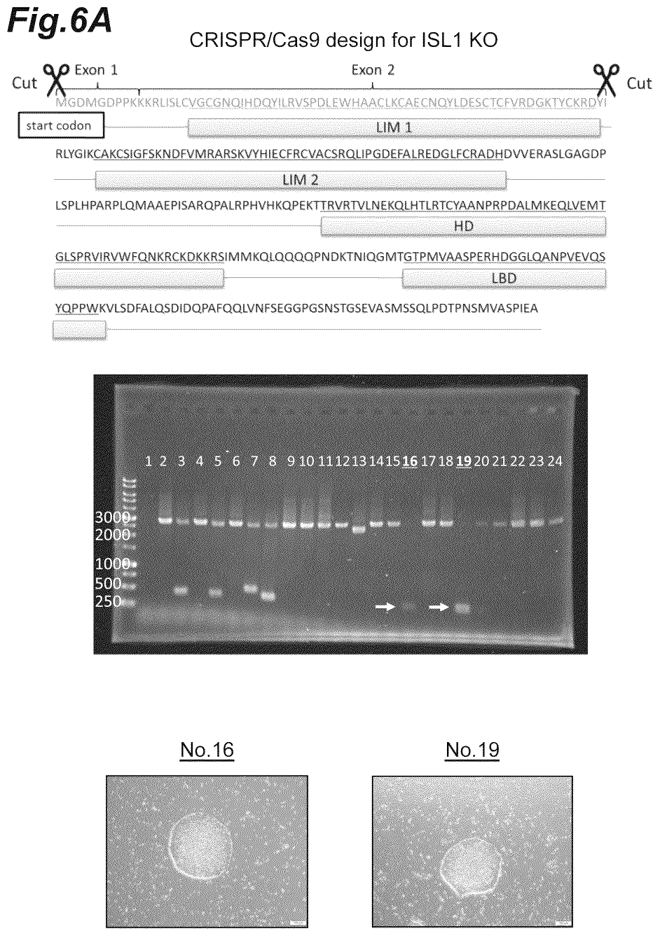

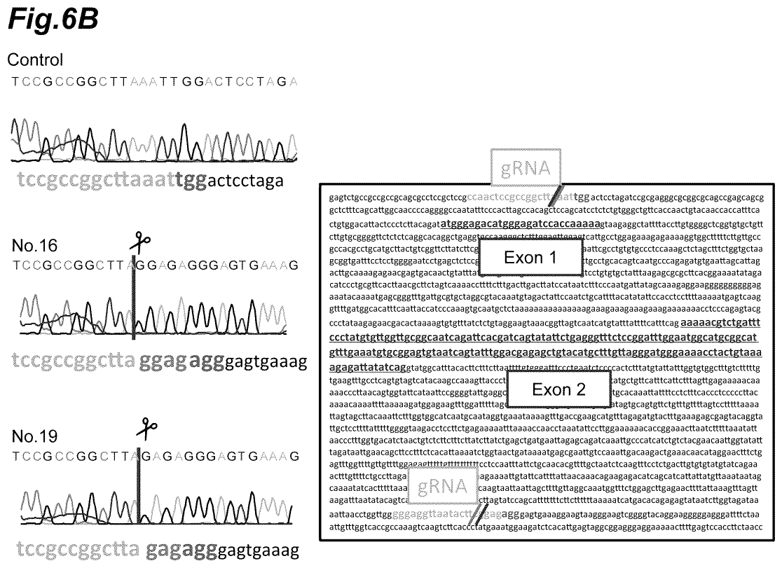

The human ES cells into which the plasmid was introduced by electroporation were inoculated in a plastic culture dish (manufactured by IWAKI & CO., LTD.) coated with Laminin511-E8 and cultured in the presence of Y27632 (ROCK inhibitor, 10 UM) under feeder free conditions in StemFit medium. When a 6-well plate (manufactured by IWAKI & CO., LTD., culture area 9.4 cm²) was used as the plastic culture dish, the inoculated number of the above human ES cells dispersed into single cells was 1×10³ cells/well. One day after inoculation, the medium was replaced with Y27632 free StemFit medium. Thereafter, the medium was replaced with Y27632 free StemFit medium once every 1 to 2 days. Thereafter, the cells were cultured until 6 days after inoculation or until 1 day before reaching subconfluence. 0.5 ng/ml or 0.4 ng/ml of puromycin was added to the human ES cells of 1 day before reaching subconfluence cultured under the above feeder free conditions, and the plasmid-introduced cells were selected. The colonies survived by selection were picked up and genetically analyzed to establish ISL1 gene-deleted lines (No. 16 line and No. 19 line, lower row in FIG. 6A). At the time of picking up the colonies, the cells were dispersed into single cells using TrypLE Select and subsequently a half thereof was inoculated in a plastic culture dish (manufactured by IWAKI & CO., LTD.,) coated with Laminin511-E8 and cultured in the presence of Y27632 (10 μM) under feeder free conditions in StemFit medium. The other half was collected as a genetic analysis sample.

The deletion at the sites of interest in the ISL1 gene of the human ES cells in the colonies picked up was confirmed by PCR and agarose gel electrophoresis (middle row in FIG. 6A) and by investigating the nucleotide sequence information (FIG. 6B).

### Example 7: Example of Differentiating the Human ES Cell Line Wherein the Function of an ISL1 Gene is Deleted into the Retina

The ISL1 gene-deleted human ES cells (KhES-1 line derived, No. 16 line and No. 19 line) prepared in Example 6 were cultured in StemFit medium under feeder free conditions until 1 day before reaching subconfluence. The human ES cells of 1 day before reaching subconfluence were cultured for 1 day in the presence of SB431542 (TGFβ signaling pathway agent, 5 μM) and SAG (Shh signaling pathway agent, 300 nM) (Precondition treatment) under feeder free conditions.

The ISL1 gene-deleted human ES cells prepared in Example 6 were washed with PBS, subsequently treated with a cell dispersion using TrypLE Select, further dispersed into single cells by pipetting procedure, and subsequently the human ES cells dispersed into single cells were suspended in 100 μL of a serum-free culture medium so as to be 1.2×10⁴ cells per well of a non-cell adhesive 96-well culture plate (product name: PrimeSurface 96-well V-bottom plate, manufactured by Sumitomo Bakelite Co., Ltd.) and suspension cultured at 27° C., 5% CO₂. For the serum-free culture medium (gfCDM+KSR) at this operation, a serum-free culture medium wherein 10% KSR, 450 μM of 1-monothioglycerol, and 1×Chemically defined lipid concentrate were added to a 1:1 mixed solution of F-12 medium and IMDM medium was used.

At the initiation of suspension culture (day 0 after the initiation of suspension culture), Y27632 (ROCK inhibitor, final concentration 20 μM) and SAG (Shh signaling pathway agent, 300 nM or 30 nM) were added to the above serum-free culture medium. On day 3 after the initiation of suspension culture, 50 μL of a culture medium comprising exogenous recombinant human BMP4 (product name: Recombinant

Human BMP-4, manufactured by R&D) at a final concentration of 1.5 nM and not comprising Y27632 and SAG was added.

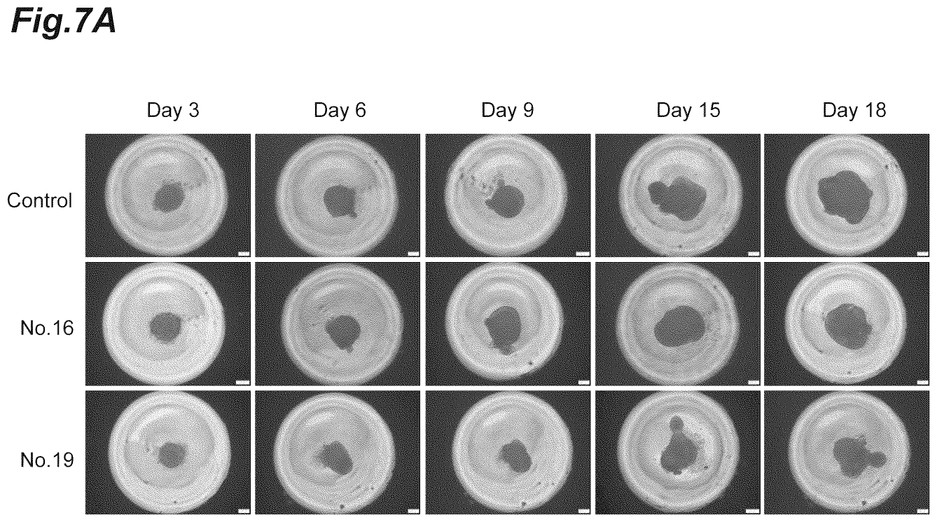

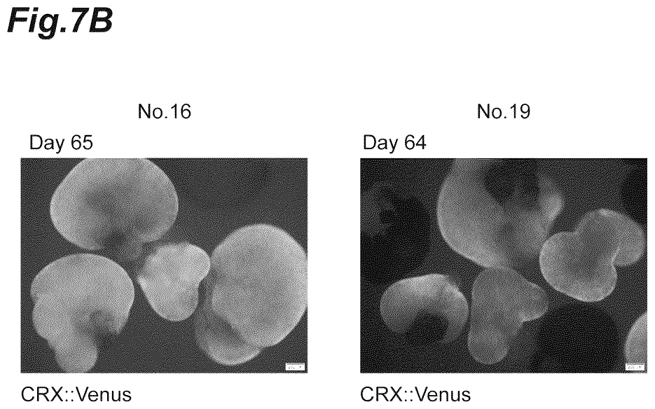

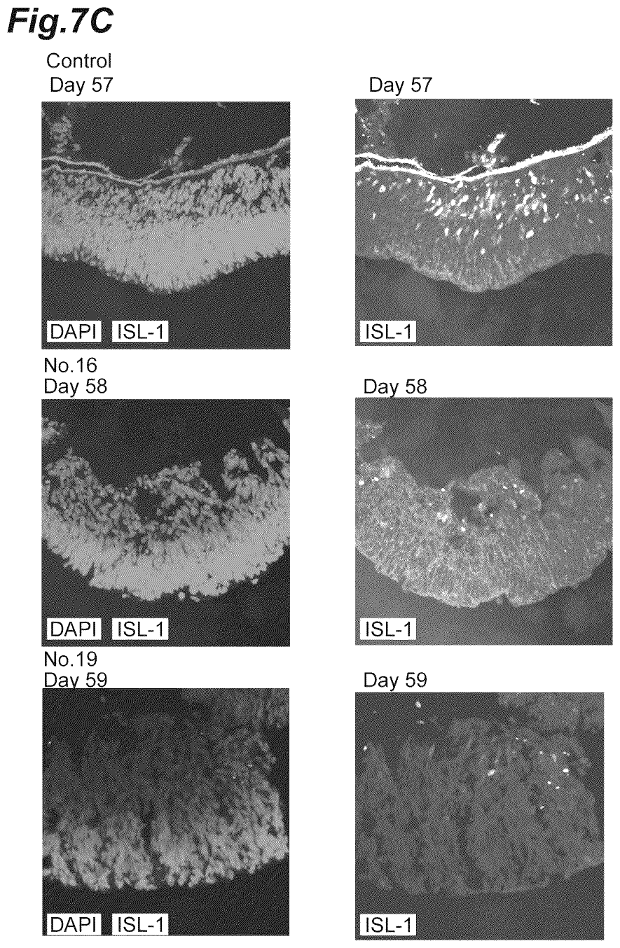

Bright-field observation was performed on the thus prepared cells on days 3, 6, 9, 15, and 18 after the initiation of suspension culture using an inverted microscope (FIG. 7A). As a result, it was revealed that cell aggregates having a stratified structure were formed also in the human ES cell lines wherein the function of the ISL1 gene was deleted (FIG. 7A).

The aggregate on day 18 after the initiation of suspension culture was moved to a 90 mm low-adhesion culture dish (manufactured by Sumitomo Bakelite Co., Ltd.) and cultured in a serum-free culture medium comprising Wnt signaling pathway agent (CHIR99021, 3 μM) and FGF signaling pathway agent (SU5402, 5 μM) (a culture medium wherein 1% N2 Supplement was added to DMEM/F12 medium) at 37° C., 5% CO₂ for 3 to 4 days. Subsequently, the aggregate was long-term cultured in a 90 mm low-adhesion culture dish (manufactured by Sumitomo Bakelite Co., Ltd.) in a serum culture medium not comprising Wnt signaling pathway agent and FGF signaling pathway agent (a culture medium wherein 10% fetal bovine serum, 1% N2 Supplement, 0.5 μM retinoic acid, and 100 μM taurine were added to DMEM/F12 medium). Half medium replacement was performed once in 2 to 4 days with the above serum culture medium. The cells were observed on days 64 to 65 after the initiation of suspension culture using a fluorescence microscope (FIG. 7B). As a result, it was revealed that three-dimensional retina having CRX-positive photoreceptor progenitor cells was formed in the human ES cell line wherein the function of the ISL1 gene was deleted (FIG. 7B).

Cell aggregates on days 57, 58, and 59 after the initiation of suspension culture prepared using the human ES cells wherein the ISL1 gene was deleted as a starting material was fixed in 4% PFA to prepare frozen sections. These frozen sections were immunostained using an anti-ISL1 antibody (product name: Anti Islet-1 Antibody, manufactured by DSHB). These immunostained sections were observed using a confocal fluorescence microscope. As a result, it was revealed that ISL1 protein was not expressed in the cell aggregates prepared from the human ES cells wherein the ISL1 gene was deleted (FIG. 7C).

These results revealed that retinal tissue can be formed from the human ES cell lines wherein the ISL1 gene has the failed function and that ISL1 protein is not expressed in the human ES cell line wherein the ISL1 gene has the failed function.

### Example 8: Example of Transplanting the Retina into which the Human ES Cell Line Wherein the Function of an ISL1 Gene is Deleted is Differentiated and Confirming the Take Thereof

The ISL1 gene-deleted human ES cells (KhES-1 line derived) prepared in Example 6 were cultured in StemFit medium under feeder free conditions until 1 day before reaching subconfluence. The human ES cells of 1 day before reaching subconfluence were cultured for 1 day in the presence of SB431542 (TGFβ signaling pathway agent, 5 μM) and SAG (Shh signaling pathway agent, 300 nM) (Precondition treatment) under feeder free conditions.

The ISL1 gene-deleted human ES cells prepared in Example 6 were washed with PBS, subsequently treated for dispersion using TrypLE Select, further dispersed into single cells by pipetting procedure, and subsequently the human ES cells dispersed into single cells were suspended in 100 μL of a serum-free culture medium so as to be 1.2×10⁴ cells per well of a non-cell adhesive 96-well culture plate (product name: PrimeSurface 96-well V-bottom plate, manufactured by Sumitomo Bakelite Co., Ltd.) and suspension cultured at 37° C., 5% CO₂. For the serum-free culture medium (gfCDM+KSR) at this operation, a serum-free culture medium wherein 10% KSR, 450 UM of 1-monothioglycerol, and 1×Chemically defined lipid concentrate were added to a 1:1 mixed solution of F-12 medium and IMDM medium was used.

At the initiation of suspension culture (day 0 after the initiation of suspension culture), Y27632 (final concentration 20 μM) and SAG (Shh signaling pathway agent, 300 nM or 30 nM) were added to the above serum-free culture medium. On day 3 after the initiation of suspension culture, 50 μL of a culture medium comprising recombinant human BMP4 (product name: Recombinant Human BMP-4, manufactured by R&D, final concentration 1.5 nM) and not comprising Y27632 and SAG was added.

On day 6 after the initiation of suspension culture of the thus prepared cells, 60 μL of the culture medium was removed and 90 μL of a fresh culture medium was added. Further, on days 9, 12 and 15 after the initiation of suspension culture, 85 μL of the culture medium was removed and 90 μL of a fresh culture medium was added.

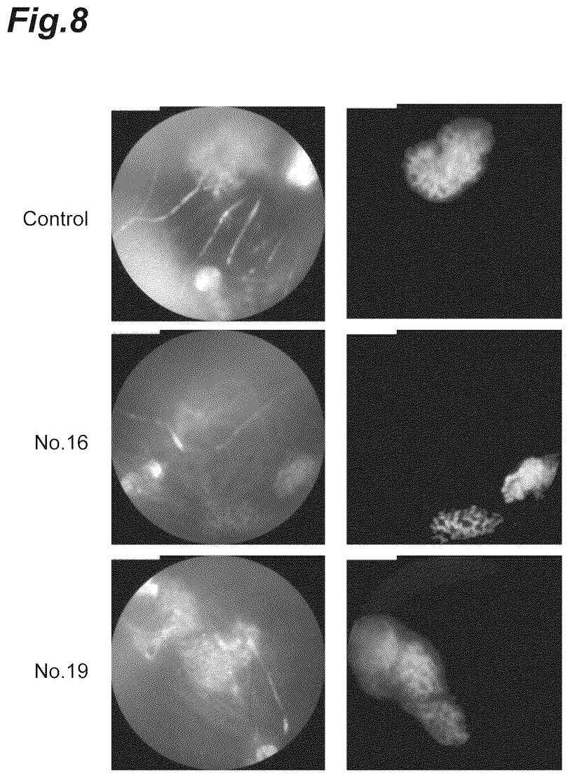

The aggregate on day 18 after the initiation of suspension culture was moved to a 90 mm low-adhesion culture dish (manufactured by Sumitomo Bakelite Co., Ltd.) and cultured in a serum-free culture medium comprising Wnt signaling pathway agent (CHIR99021, 3 μM) and FGF signaling pathway agent (SU5402, 5 μM) (a culture medium wherein 1% N2 Supplement was added to DMEM/F12 medium) at 37° C., 5% CO₂ for 3 to 4 days. Subsequently, the aggregate was long-term cultured in a 90 mm low-adhesion culture dish (manufactured by Sumitomo Bakelite Co., Ltd.) in a serum culture medium not comprising Wnt signaling pathway agent and FGF signaling pathway agent (a culture medium wherein 10% fetal bovine serum, 1% N2 Supplement, 0.5 μM retinoic acid, and 100 μM taurine were added to DMEM/F12 medium). Half medium replacement was performed once in 2 to 4 days with the above serum culture medium. Three-dimensional retina was cut off on days 58 to 65 after the initiation of suspension culture and subretinally transplanted using a syringe into a retinal degeneration rat, a photoreceptor cell degeneration model. On months 1 to 2 after transplant, the take of a graft was observed based on the fluorescence of CRX::Venus using a fluorescent funduscopy (product name: MicronIV, manufactured by Phoenix research). As a result, the retinal tissue wherein the ISL1 gene was deleted, when transplanted, was subretinally taken into the retinal degeneration rat as comparable as the retinal tissue wherein the ISL1 gene was not deleted (FIG. 8). Thus, it was revealed that the retinal tissue into which the human ES cells wherein the ISL1 gene is deleted are differentiated is taken after transplant.

### Example 9: Example of Transplanting the Retina into which the Human ES Cell Line Wherein the Function of the ISL1 Gene is Deleted is Differentiated and Confirming the Reduction in Ratio of Bipolar Cells

The ISL1 gene-deleted human ES cells (KhES-1 line derived) prepared in Example 6 were cultured in StemFit medium under feeder free conditions until 1 day before reaching subconfluence. The human ES cells of 1 day before reaching subconfluence were cultured for 1 day in the presence of SB431542 (TGFβ signaling pathway agent, 5 μM) and SAG (Shh signaling pathway agent, 300 nM) (Precondition treatment) under feeder free conditions.

The ISL1 gene-deleted human ES cells cultured above were washed with PBS, subsequently treated for dispersion using TrypLE Select, further dispersed into single cells by pipetting procedure, and subsequently the human ES cells dispersed into single cells were suspended in 100 μL of a serum-free culture medium so as to be 1.2×10⁴ cells per well of a non-cell adhesive 96-well culture plate (product name: PrimeSurface 96-well V-bottom plate, manufactured by Sumitomo Bakelite Co., Ltd.) and suspension cultured at 37° C., 5% CO₂. For the serum-free culture medium (gfCDM+KSR) at this operation, a serum-free culture medium wherein 10% KSR, 450 UM of 1-monothioglycerol, and 1× Chemically defined lipid concentrate were added to a 1:1 mixed solution of F-12 medium and IMDM medium was used.

At the initiation of suspension culture (day 0 after the initiation of suspension culture), Y27632 (final concentration 20 μM) and SAG (Shh signaling pathway agent, 300 nM or 30 nM) were added to the above serum-free culture medium. On day 3 after the initiation of suspension culture, 50 μL of a culture medium comprising recombinant human BMP4 (product name: Recombinant Human BMP-4, manufactured by R&D, final concentration 1.5 nM) and not comprising Y27632 and SAG was added.

On day 6 after the initiation of suspension culture of the thus prepared cells, 60 μL of the culture medium was removed and 90 μL of a fresh culture medium was added. Further, on days 9, 12 and 15 after the initiation of suspension culture, 85 μL of the culture medium was removed and 90 μL of a fresh culture medium was added.

The aggregate on day 18 after the initiation of suspension culture was moved to a 90 mm low-adhesion culture dish (manufactured by Sumitomo Bakelite Co., Ltd.) and cultured in a serum-free culture medium comprising Wnt signaling pathway agent (CHIR99021, 3 μM) and FGF signaling pathway agent (SU5402, 5 μM) (a culture medium wherein 1% N2 Supplement was added to DMEM/F12 medium) at 37° C., 5% CO₂ for 3 to 4 days. Subsequently, the aggregate was long-term cultured in a 90 mm low-adhesion culture dish (manufactured by Sumitomo Bakelite Co., Ltd.) in a serum culture medium not comprising Wnt signaling pathway agent and FGF signaling pathway agent (a culture medium wherein 10% fetal bovine serum, 1% N2 Supplement, 0.5 μM retinoic acid, and 100 μM taurine were added to DMEM/F12 medium). Half medium replacement was performed once in 2 to 4 days with the above serum culture medium. The obtained three-dimensional retina was cut off on days 58 to 65 after the initiation of suspension culture and subretinally transplanted using a syringe into a retinal degeneration rat, a photoreceptor cell degeneration model. On the age equivalent to 240- to 270-day differentiation after transplant, the ocular tissue was fixed in PFA and replaced with sucrose. Tissue sections were prepared using a cryostat. Human cells, bipolar cells, and amacrine cells in the tissue section were stained by immunostaining using respectively an anti-Nuclei antibody (product name: Anti-Nuclei, manufactured by Millipore), an anti-Chx10 antibody (product name: Chx10 Anti Antibody, manufactured by EX alpha), and an anti-Pax6 antibody (product name: Anti Pax6 antibody, manufactured by Covance Inc.) and the grafts after transplant were evaluated. Three slides each of the retinal tissue wherein the Islet-1 gene was not deleted (wild type) and the retinal tissue wherein the Islet-1 gene was deleted were stained and 2 areas were photographed per slide.

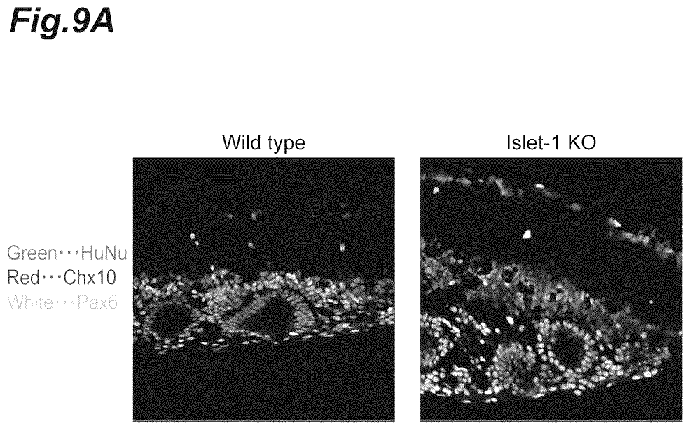

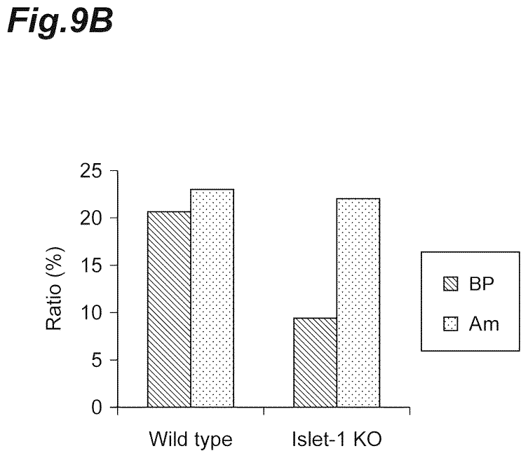

Fluorescence observation was performed on the stained tissue using a confocal microscope (product name: TCS SP8, manufactured by Leica) and ratio of the bipolar cells and the amacrine cells derived from the human cells in the grafts were investigated in 3 sections at 2 areas. Representative photographs thereof are shown in FIG. 9A. From FIG. 9A, it was confirmed that the ratio of the Chx10-positive cells (i.e., bipolar cells) was reduced when the retinal tissue wherein the Islet-1 gene was deleted was transplanted compared with the case wherein the wild type retinal tissue was transplanted. Additionally, graphs of the numericalized ratio are shown in FIG. 9B. The same results were also confirmed by FIG. 9B. More specifically, it was revealed that the ratio of the amacrine cells (Am) to the all human cells remained substantially unchanged, whereas the ratio of bipolar cells (Bp) was reduced when the retinal tissue wherein the Islet-1 gene was deleted was transplanted compared with the case where the retinal tissue wherein the Islet-1 gene was not deleted was transplanted. The reduction in ratio of the bipolar cells derived from the graft (human derived) enhanced the contact ratio of the photoreceptor cells derived from the graft to the bipolar cells on the host side (mouse side), suggesting that the functional take of the retinal tissue after transplant was improved.

### INDUSTRIAL APPLICABILITY

According to the present invention, the retinal tissue suitable for transplant can be obtained.

1: A cell population for transplant, comprising retinal cells with a modified bipolar cell-regulating gene, wherein the bipolar cell-regulating gene is a BHLHE23 gene.

2: The cell population for transplant according to claim 1, wherein the cell population is in a form of cell suspension or cell aggregate.

3-5. (canceled)

6: The cell population for transplant according to claim 1, wherein the BHLHE23 gene has a nucleotide sequence set forth in the following (1) or (2):
(1) a nucleotide sequence set forth in SEQ ID NO: 10 or 13,
(2) a nucleotide sequence having a nucleotide sequence modified from the nucleotide sequence set forth in SEQ ID NO: 10 or 13 by deletion, addition, insertion, or substitution of one or a plurality of nucleotides, and encoding a protein having 80% or more sequence identity with an amino acid sequence set forth in SEQ ID NO: 12 or 15, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 12 or 15.

7: The cell population for transplant according to claim 1, wherein the modification of a bipolar cell-regulating gene comprises deletion of the gene.

8: The cell population for transplant according to claim 1, wherein the retinal cells are derived from a pluripotent stem cell.

9: The cell population for transplant according to claim 8, wherein the pluripotent stem cell is an induced pluripotent stem cell or an embryonic stem cell.

10: The cell population for transplant according to claim 1, wherein the retinal cells comprise one or a plurality of cells selected from the group consisting of a retinal progenitor cell, a photoreceptor progenitor cell, and a photoreceptor cell.

11: The cell population for transplant according to claim 10, wherein the retinal cells comprise one or a plurality of cells selected from a Chx10-positive cell, a Crx-positive cell, and a recoverin-positive cell.

12: The cell population for transplant according to claim 10, wherein the sum of the number of retinal progenitor cells, photoreceptor progenitor cells, and photoreceptor cells is 10% or more of the total number of cells in the cell population for transplant.

13: The cell population for transplant according to claim 10, wherein the functional take rate after the transplant of the photoreceptor cells or photoreceptor cells induced from the retinal progenitor cell or photoreceptor progenitor cell after the transplant is improved.

14: A culture of a cell population for transplant, comprising:
(1) a cell population for transplant according claim 1; and
(2) a medium necessary to maintain viability of the cell population for transplant.

15: A method for producing a cell population for transplant comprising retinal cells, comprising the following steps (1) and (2):
(1) modifying a bipolar cell-regulating gene of pluripotent stem cells to obtain in vitro, a cell population comprising the pluripotent stem cells with the modified bipolar cell-regulating gene, wherein the bipolar cell-regulating gene is a BHLHE23 gene;
(2) inducing differentiation of the cell population comprising the pluripotent stem cells obtained in step (1) into retinal cells in vitro to obtain a cell population for transplant comprising the retinal cells.

16: The method of production according to claim 15, wherein the cell population for transplant comprising the retinal cells is in a form of cell suspension or cell aggregate.

17-19. (canceled)

20: The method of production according to claim 15, wherein the BHLHE23 gene has a nucleotide sequence set forth in the following (1) or (2):
(1) a nucleotide sequence set forth in SEQ ID NO: 10 or 13;
(2) a nucleotide sequence having a nucleotide sequence modified from the nucleotide sequence set forth in SEQ ID NO: 10 or 13 by deletion, addition, insertion, or substitution of one or a plurality of nucleotides, and encoding a protein having 80% or more sequence identity with an amino acid sequence set forth in SEQ ID NO: 12 or 15, wherein the nucleotide sequence encodes a protein satisfying at least one of:
(a) the protein has DNA binding ability,
(b) the protein has a function to regulate genetic transcription, and
(c) the protein can be recognized by an antibody that specifically recognizes a protein consisting of the amino acid sequence set forth in SEQ ID NO: 12 or 15.

21: The method of production according to claim 15, wherein the modification of a bipolar cell-regulating gene comprises deletion of the gene.

22: The method of production according to claim 15, wherein the pluripotent stem cells are induced pluripotent stem cells or embryonic stem cells.

23: The method of production according to claim 15, wherein the retinal cells comprise one or a plurality of cells selected from the group consisting of a retinal progenitor cell, a photoreceptor progenitor cell and a photoreceptor cell.

24: The method of production according to claim 23, wherein the retinal cells comprise one or a plurality of cells selected from a Chx10-positive cell, a Crx-positive cell, and a recoverin-positive cell.

25: The method of production according to claim 23, wherein the sum of the number of retinal progenitor cells, photoreceptor progenitor cells, and photoreceptor cells is 10% or more of the total number of cells in the cell population for transplant comprising the retinal cells.

26: The method of production according to claim 23, wherein the functional take rate after the transplant of the photoreceptor cells or photoreceptor cells induced from the retinal progenitor cell or photoreceptor progenitor cell after the transplant is improved.

27: A method for treating a disease based on a disorder of retinal tissue or a condition of retinal tissue damage, comprising transplanting an effective amount of the cell population for transplant according to claim 1 into a subject in need of transplant.

28: A pharmaceutical composition for treating a disease based on a disorder of retinal tissue or a condition of retinal tissue damage, comprising the cell population for transplant according to claim 1 as an active ingredient.

29: The pharmaceutical composition according to claim 28, wherein the pharmaceutical composition is in a form of cell sheet.

30: A therapeutic agent for a disease based on a disorder of retinal tissue or a condition of retinal tissue damage, comprising the cell population for transplant according to claim 1.

31: The therapeutic agent according to claim 30, wherein the therapeutic agent is in a form of cell sheet.


SEQUENCE LISTING (24 sequences)

SEQ ID NO: 1 - DNA, 11607 bp, Homo sapiens
  source 1..11607: mol_type = other DNA
gaaggaagaggaagaggaggagagggaggccagagccagaacagcccggcagcccgagcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaagtaagaggctattttaccttgtggggctcggtgtgctgttcttgtgcggggttctctctcaggcacaggctgaggtgccaagggctctttggagttggagtcattgcctggagaaagagaaaaggtggctttttcttgttgccgccacgcctgcatgcttactgtcggttcttatcttcgggaaactgattgtaccttgtgtgtgaattcgcctgtgtgccctccaaagctctagctttctggtgctaagcggtgatttcctcctggggaatcctgagctctccgagaaggttattatgttgcaaaggtctgcctgcacagtcaatgcccagagatgtgaattagcattagacttgcaaaagagaacgagtgacaactgtatttatgcctgctcttgctaacaatatccagtcctgtgtgctatttaagagcgcgcttcacggaaaatatagacatccctgcgttcacttaacgcttctagtcaaaaccttttctttgacttgacttatccataatctttcccaatgattatagcaaagaggaaggggggggggagaaatacaaaatgagcgggtttgattgcgtgctaggcgtacaaatgtagactattccaatctgcattttacatatattccacctccttttaaaaatgagtcaaggttttgatggcacatttcaattaccatcccaaagtgcaatgctctaaaaaaaaaaaaaaagaaagaaagaaagaaagaaaaaaacctcccagagtacgccctataagagaacgacactaaaagtgtgtttatctctgtaggaagtaaacggttagtcaatcatgtatttattttcatttcagaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggtatggcatttacacttctttcttaattttgtgggatttccctgaatctccccactctttatgtattatttggtgtggctttgtctttttgtgaagtttgcctcagtgtagtcatacaagccaaagttaccctgtacatgtgttaaaaaaatcaagctatgctgttcatttcattctttagttgagaaaaacaaaaacccttaacagtggtattcataattccggggtattgaggcttgtttaattactcttggagtttatgatgcacaaattattttcctctttcaccctcccccttacaaaacaaaattttaaaaagatggagaagtttggatttttagctttaaaatagggttgatttttgttgtatagtgcagtgttctgtttgttttagtcctttttaaaattagtagcttacaaattctttggtggcatcaatgcaataggtgaaataaaagtttgaccgaagcatgtttagagatgtactttgaaagagcgagtacaggtattgctccttttatttttggggtaagacctccttctgagaaaaatttaaaaccaacctaaatattccttggaaaaaacaccggaaacttaatctttttaaatattaaccctttggtgacatctaactgtctcttctttcttatcttatctgagctgatgaattagagcagatcaaattgcccatcatctgtctacgaacaattggtatatttagataattgaacagcttcctttctcacattaaaatctggtaactgataaaatgagcgaattgtccaaattgacaagactgaaacaacataggaactttctgagtttggttttgttgttttggagagtttttgttttttttttcctccaatttattctgcaacacgttttgctaatctcaagtttcctctgacttgtgtgtatgtatcagaaactttgttttctgccttagaaagccagtagtctctaaagaaaattgtattcattttattaacaaacagaagagacatcagcatcattattatgttaaataatagcaaaatatcactttttaaatgtccggtggctattaacaagtaattaattagcttttgttaggcaaatggtttctggagcttgagaacttttattaaagtttagttaagatttaatatacagtcacagtttgctcctgctcacttagtatccagcatttttttcttcttttttaaaaatcatgacacagagagtataatcttggtagataaaattaacctggttgggggaggttaatacttcggagagggagtgaaaggaagtaagggaagtcggggtacaggaagggggagggattttctaaattgtttggtcaccgccaaagtcaagtcttcaccctatgaaatggaagatctcacattgagtaggcggagggaggaaaaacttttgagtccaccttctaacctctgacaaatgagcgttttcattgtttactagattggtgtgtaaacgcaagattctagagaaggagagcccacttcaggagtatctttactgctatggaaatagtattttgctcaattgcacacaggcttgcatgtgcctaattctggatacacacatgtgtagaaggaactaatcatttttaccttctcttcactctctctcaactctgtgtgtgtgtgtgtgtgtgtgtgtgtgtgtgtgtgtaatcttgtagttgtaaaagcagaacagactggacagttagatttccacatctctccttggagaagcaggatgcctcctcctgttatgtggatcttttcctctctcttccattctttctgttcgtaggaatgccccagcttctgttattcctgaaagatggagaaggggccagggaagtgcagcctagatggaacctataaagattgtcccttggtaaggaaaggccaggagtgagaaagaccttagaagcgggtctttgcatttttttcattctggtcatggttttcaagaaaattgaaatgaggtagatgattcagcaacttgaaaaagattgagggaacagacgcagatttttttaaaaaaataataatacaaggaagaatggagaggaaattttctgttaacattgctgcctgaagaaaatctttagttggagaaagactggaaagtacttgtgcaaaaggagatgtggaaactctcagaggtttcattttgttattctgcttgtttatttgtgagtgtttgcaaaccgagtggggtgacaatccccttctcctacctccttttttcttggaaggaggactttttgttgcagttttagacatttctagcagcagaaattgtgggatagggaagtgaaagtgttggtgtcggtggccaccagagtctttctggattccttcctgccaagatctgcaagatcaacactgggattgattgctagagcagcagcccgagtttggaacccatcaatacattttctgtggtacaagctaggtgttttgagctaagagttaccaactaagacagaggttcatcggaaaggaaacgggagtaaaagaaagggaggagggagggaggggaaaagagagatgggggaaggaagagagacagggaaggagagagcagggtttcatttctgtccttctgtttccaacttctgtttggaaatgctgtttacttggggcgtcttgcccgggatcttgggccagggaagtgccggcctgaagtgaccccctcttcctgtacttctctccccgctctgggccgcctccgctcccccctcccccgcacaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcaggtactcctctgcccggctcgggtaggcaggcgccaggttaagccagcctgtgtgccagcggccacaacaactatggtagctacaggggtggtcgtagtgtttgcctgcagttaaatgaagtgttctgtatgcaatttgcgctgtgctctgctcctttgcagcaaggttcaatgcactcactgtctcccttgattccccgagcacacctacaccgtctgtgtgtctctatatggttacacataaatgtacaccacttgtgtacacgtgtatacacacgcccaaacattacttccagttcgctctggcctccaaaccttggcttgctgaaaacgggcttcagctcccagccaggtattctcctgctgcctaattaaaggggcggagccccgggtccctggagcttcatcctttaacccaatgaaggaagcttaggtggcctgaagtcatttagtctcccaaatcctttttccttgtgagttgcttcacactcgaaattttttttttaatttttttatctttctgtgagagaacaggactgaaaagatacagttttaaaaactgcaggccattgcacagagttgtaatataaaactgtcaacaagcttatctgcagtaattgccttttaaagggagcctgcttctttaaatcattcattctatatgatttggtgagaatttcatcttcaggcccatggttgtagctctaaattgaccccataggtgttggcctgaccctagggggttgtagaaggtgcaggatttgtatcatgtagataagaggactcattcccaaggaagaggagtggaaacacagcaaggttggccgggaccaaagcagtgggttagaaggtggacagtgtttccaaacctgacttcctgccatgaatagatctacccctttgcagttttaaagtatcaattcccactaaacactgaaggtgaggaaactatagccctcccttacccttctgccttctggcagctctaagaattctgttcagggggatttgtgactagtttgcaccggggcacggctggggtggtgctcctgttcagtggagcctgcactctgcttgtggggaagcacagaggaagctaaaataccgagagggaggcgggggacatctcccagccaccgtttatctagagcctaggcagctcaacagagtttccgttttccactgcttgggatcagcccatctcaggaacatccatgtattaccttagatttaatactaagagcagggattggagatatggcagaaatagcgaatctcttcagccccttcacatgactgtcctctcggactgaagttcaaggcgttctggcagagttctcgaccttccccttgcagaagtccctgctggtgtagtatttatggctgtcactgaagtgctctgcgttcctttccctggtaccctctgtggccttggcccaagagaaaattctgatcctggagagggtggtaatcaatgtaactggggcccagtctgggcacaaggaaaggtgagaatggaggagaaacagtgctgaaaaatgccacccctgctgtgaacagggggacagactttgagacctgcttcccttggctaacactttgttgacacgaggaggggcgagtgctgcgtttcaggccgggattactcagcaaagacctctgcagattagagaggaagattttattctccctttcaccctcttcgcccccacctctgccgccccctgctttgtgtgctgaggctgcaaaccctagccattgtcctgagtatctcgggcgggcgagcaagtaagcgggcgggcgggcgggcaagcgagcgagcgagcgagcgcgcgaccgcgggcgggccggcaagcgagcctccagcccagcgctcacggcgctccttgccccgcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactgtgagtggctctggggccgggcagggaatgcgagggggaaggagacgcagcgtgcgaggtgcgttcctggtacgcaggatcgcacggttttcaatcctgctcctgggcaggagtttggccggggctgcccctcatccttacccccctacccatgccccgggggacaggctacccggcgccggccgccagctgagggcggggaagctgggaggctccgtgcgccgggggagcagcatccaggtcccaacctcgtgggtgggctcatgcccttccacctcgcctgtacctgtgaaccggagaaacgccgtcctcccctctgagggcaggcggcaacgaggtttggcccgggttttgccaacattcagatcgtcagttcctcacgtacacaagaagagggagggataataccttggattcctgcctacatccaggggttccgtgggcaggtcaccctgtgagcccccagggcgcaccgcacttctaagtaaggtcggccgctgcgccttcaggctggcgagttcccccaaggtgacccgcatgcccagatcaccctctgctccaggtgaagcccaggcctccacagaggcatcaggcccctcgcaccagtatccactgttatcttggtcccacggaagcacccactctgcaggcctcctggtgaagttaagctagagtttcttttcttcctttttttcttttctttctttttcttttttttttttttttttttttttttttttttttttttactgctttggacctatttttaaatgccataaaatctgctgtcattaaacttggcaggctggccaagattgggccagggcactttctgagttggttagtgcataatagcacaataggaaccagacccaaatgctttggggggatggagtggggggctggctcttccttgaggagaacggcttggaaaaaatctgcagctaactgaaactgctcagaaaaccaccctgtctagaggctgaagggaagccctgcttacctcagctttttagttctgggaagctatggtctgagaaggcagaggggaggaattgggctgagctgtgaaggtaagggggaagaagaaaatcaaagtagaatttggtttaataaggtccatgcagacctaatagtccagcccacagaggcagaaaaacaaaacaataaaacaaattgaattctaactaatatccgtaggtacggcggattaactgagtcaataaagaccactatatagataagataataccagggtatatttgcttagcctgtgcagacaacggagggagggaatttgctcattaacatgttgggattggttggggggcctattcacagaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtaagtgtcagctcccagatggaagaggctgaattcccaacaggagactctggtttaactgtcacacattgaaagattcagtggggagggtgccttcttgggctcagggttggggagaaaccaaggaggtgggtaatgaagagaagggagacaaatgcagggaaaacgaacctcttggcatctttttttttttaatgagactgcataatttgaccatataggttgaattttctatcaatcaggccttctttgaaggattaatttcaaggtacctaactctaggtagcatgtgccagaagatgtacagtgttggagaatcatacatcttagaattttagagttgtcaaggacttcaggaaatcgtcttggcatttcaatcagcaattagtaagtttatccttcctgagcatctagagaatgggatatataggacccaaatcaaggcgattgtagtatataatcaatgctataatacacaatcagtgttgttatcaataaacagcaggcatgtgtctgggtacaattttcaaatatattaataaagattattcaaatagatgaatactttttgttacagtatcccttgctgggaatgtcttaatctaaaatgtaggaccgtttaaatgttttcaagtgtatgagttcaaatgtcatagagacacacagtgtgtaccatgtatagcaaaagggacatgagctctaccaatcagaagtaaagtgtttaatctgtgaaaaccttaacatgttttccacatccagagaggagaaaattaattcactttttgcctacaaaaggcttaaggggtcaagataaataagaacaataaatatatgtcctttgtaatatgctatatttatatagatgatttttttttcttaaagagtaatcagccttatagaatcttgttttataaaatgtaaagatctatcctgaaaccttgttcccttttttggaaatgaagctttagttgaggttagctttttaccctcatatttacctggagggcatttgctttctcaatgtcaacagttaggtaattggccagaggcaagtggttaaaagggcttggccccaggcttgtgtttgcaaatgctaagtgggtgcagaggctagaagtcccttaatctcatattggaaaaatttactgtagaaagaaatgtaggctctagaactaggaaaaaaaaattattctaagctcattaatctgttgagttatttgagcgaatcctgaatcacaggaggaaggtaaggggaggcttcagggcagccaaatgtttgcactttctgaaactttagtgtcagatgagagcagtggaagggaagctgaggcaggagtgggcatagttagagaaggtttacaacagcagtacaatgcgtttagggttaaaagaaggagtcagatatttaagaaggagtcagatattagggttaaaagaaggagtcagaatgggatgatgtcataatatatgggtctcattttggaaggaagagcctgatttaaagagagagagagagaaaggccaagaggcagcaggaccaacaaggaagaatgcccaagctgtgagcctgctgaggagttaatctttgttctgtggagcctcctctcaatctcctgtcaaaggatctgagcctgttacggattttccaactgaagaagagagtctttgatgcctagagactgagagctcacctactcccagggcaacatgtagccagcaggataattttatttcgagcatgcatagtagagttgtgatgccattttacagtgggaaacacatttgttcttaaataatttaatgcaacataatgttgggaattcagtttcagttaaaacagagatcttttggaagatgggaaagtgagaggatttcttcccaagtttttctcctctaggctttctctaagcctgttaaaattcagttatctatgtgaatatctttacatatctatctacacaaacatttctacatatacaatatgatgagtttataatctttttatgaatactattccagtgtcctttatttatttctcaaccttctatgcaggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattatatgtctaattctatgtgttttgtctttttcttaaatattatgtgaaatcaaagcgccatatgtagaattatatcttcaggactatttcactaataaacatttggcatagataaataaataaa

SEQ ID NO: 2 - DNA, 2729 bp, Homo sapiens
  source 1..2729: mol_type = genomic DNA
  CDS 549..1598
gaaggaagaggaagaggaggagagggaggccagagccagaacagcccggcagcccgagcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattatatgtctaattctatgtgttttgtctttttcttaaatattatgtgaaatcaaagcgccatatgtagaattatatcttcaggactatttcactaataaacatttggcatagataaataaataaaaaaaaaaaaaa

SEQ ID NO: 3 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 4 - DNA, 11465 bp, unidentified
  source 1..11465: mol_type = genomic DNA
caatggcgatggagctgagttggagctgagaagtttgagtaagagataaggaagagaggtgccccgagccgtgcgagtccgccgctgctgctgcgcctccgctctgccaactccgccggcttaaatcggactcccagatctgcgagggcgcggcgcagccgggcagctgtttcccccagttttggcaaccccgggggccactatttgccacctagccacagcaccagcatcctctctgtgggctattcaccaattgtccaaccaccatttcactgtggacattactccctcttacagatatgggagacatgggcgatccaccaaaaagtaagaagcggtttaaccttgtggggcttggtgtgctgttgtggtgtgggggggtctctctcaggcaccgatgacagggctctttggatttgtagtcactgcctggagagacagaagagaaggtggtccttttgttgttgttgttgctgctgctgttgtgtgtgtgtgtgtgtgtgtgtgtgtgtgtgtgtgtgtgtgtgtgtgtgtgtgtgttttcttcttgtcacgcttgcagcttgcatgtttggtgtgaattccgatctttgagaaactgatcataccttgtgtttgaatgctctgtgccctcccaaactctagccgcctgcccggtgcttagcggtgatttcctcctccggattccggagctcttggagaagttattatgtagcaaaggtctgcctgcacaaaccacaccggcagatatgaattagcattagacttgcaaaagagagcgagtgacaactgtattgatgtctactcttctaacaatatccattcccgcgtgctattgaagaacgtgcttcacggaaaatatagacatcccagcgttgctttaacgcttctagtcaaaaccttttctttgacatgacttatccataatcttgcctaatgattatagcaaagagggaggagggagaaaacacaaaatgagcgggtttgattgcgtgctaagcttacaatgtagactattcaaatctgcgttttacatatattccacctccctttttaaaaaagcgagtcaaggttttgatggcacactccaattaccatcccaaagtgcaatactctttaagggaagaacaagaaaaaaaaaaaaagggaaagaaaaaaattcttccagaatacaccctataagagaacgacactaaaagtgtgtttatctctgtaggaagtaaacggttagtcaatcatgcatttattttcatttcagaaaaacgtctgatttccctgtgtgttggttgcggcaatcaaattcacgaccagtatattctgagggtttctccggatttggagtggcatgcagcatgtttgaaatgtgcggagtgtaatcagtatttggacgaaagctgtacgtgctttgttagggatgggaaaacctactgtaaaagagattatatcaggtatgccatttttacagggttattttctgagatttcttggctctccttgtttattatttgatgtgcctttgttttgttttgttgtttttagagggagggagaacgtttactttagagtgcccagtgtacaagaaatgtttttaaaattaaactgttgttcatttcatatctttggggggtggggtgtcgctgtgtttcataaatttttgttgataaggtttgttaatgactctttgaatttttcatacaaaagattatttccctctttaaacttccctaaggacaacaacaaaattaaggcaaataagtttagggtttcaaactttaaaaattctattttcactgtatagtttatcatcttatttatttttctcctgttttatcaatgacttacattattttgggggctccaaataaacagacaaaagtttcaccaaaggatttttgaagaacaagtacaaacattgctccctttacttttggggtgactcttcctttgataaaaaattttaaaaccaaaataaaatattccttaacgccccccccccaaaaaaaaccaccggaaatttaatctttttaaatattaaccctttggtgatatctaactgccttttctttcttatcttatctgggctgatgaattagacagagcagatcaaattgcccatcatctgtctgcgaacaattggtatatttaggtaattgaacagcttcctttctcacattaaaatctagtaactgataaaatgagggaatcagctgacggacaagaccaaaaccagggaggaggatctgagctggcttttgttgcacttttttttttttcctaatttcaagtttctctgacctaccagtttatatcagaaacttgactttctaccttagaaagcctggtagaatttctaaaggaaaatgtgtttgttctaatacaaagcaagatactgttccccttcactgttaggctggcgatggcagaaatgtagccgattttagatttcagaggtggccattggcaagtgatcggcttttcctaagccataaagcgctttggagcttcagaactttcattaaagtttagttaagatttaaaatacagccccaccagtttttctcctgctggaataatgcccctccctcggttttgttttgttttgctttaaaaaaaacatagattggaggatatattcatgatagctaaaattaaacaatatactagggagggtttgcttgtttgtttgtttggtttggttttaatgacaaggagaatgggaggaagtgggatacaggaaaggggagggatttcgtcaatggtttgggaacccggcaaagtaagtcttccttctataaaatggatgatcttgcattggagaggcagaaggtaggaaaaaaaatttcaagtctgtcttccaacctctgagaaataatcccttttcatcttttacttggctggtgtgggctctcgggacactggagagggagaagggagaacctactttgaaactccaaaagcatctttacgcctctgaagatactgtttccctcccttgtatacagacttgcttcattccggatctgtcaatttgcagatggagtcactcagttacttttccttttctcttctcttctcttccgcctgggagcatgtttgtaagtgtgtgtgtgtgtgtgtgtgtgtgtgtgtgtgtgtgtgtaacacccatgcacaacccttttggaggatctatagttgtaaaagcagggtagagtgaccatttttattttaaggtctctcattggggaagcaacatgccacctcctttgtgtggatcttgtcctatcttttttcactctctatgtagaaattactgatagctattgagggatatccaggcaatgaagcagtggatggactataaggactataaggactgagcggtggtaaggaaaggctgacaggggaggaagcttttagcagtgcgggttggttttgttgtttttttgtccatttgtcttgctagccttgtcccccaggcaaactgaagtagaggtacatgctttaggaaccgaggaaagactggttggaggaacagaagctgggcattaaaataaaagaggaagaatggagaaagaactttccattaacaagagccccgtggaaaatctttagttggataaatactggaaggccttcactggagaggacctggaaactctctagtgcttcatcttgttattgcgttctgggattttgtgtgttcagatggagtggggtgaccctcagtccccttccccacctttgttgttgatggtgatgatggtggtggtggtggtggtgtgctttttcagaaatacttttagtagcagaaattgagggatagagaatggaaagcatagggctactggccactaatgtcccctgaatccctccctgcaagtatttgcaagaataatcctgagactggtaccagaggctggttcgaggtttagaatccatcaataaattttccttggtttgggctgagtgcctgaactaagaggtacaaacttagacagacacagaggttcaccataaaggttccccttgttaaaggggaggttaaggaagaaggagggagaggaagagaagggggaggagggaggaagggggtggggttgtttctatattctgcccaactttgctattgctatttttttttccaggcaggtcaggtttatggcccgggtctctggccatcttgggacagaacctgcctgaaatcacccatttttgtgtgtgtgtgtgtgcttttcttcccatgccggggccggttcattcaggttgtacgggatcaaatgcgccaagtgcagcataggcttcagcaagaacgacttcgtgatgcgtgcccgctctaaggtgtaccacatcgagtgtttccgctgtgtagcctgcagccgacagctcatcccgggagacgaattcgccctgcgggaggatgggcttttctgccgtgcagaccacgatgtggtggagagagccagcctgggagctggagaccctctcagtcccttgcatccagcgcggcctctgcaaatggcaggtactcctccacccagaggctgagaaaaggcaggcggggctaaatcataacctttctttcctgcaacctaggggtcacacagtggtgcagaaagtggcctgcagtaaaccacacgtgctctttgcctttctagcaaatttccatacactcgtcgtctgccttgacggcacaaccacacatctaaatgtctgtctatatacaagtacacagcataccccgcttgtgcacacatgtatacacaagcccaaactgtactgtcagctttactttggcctctaacccatagcttactgaaaatgggcttcagctctctgccaggttttctcctagctgccgcctaattaaaggggtggagccctaggtctcgaggagtttcctccctagccctgcgaaggaagcttaagcagccccaaggcatttagcttcccacctctctctctctctctctctctctctctctctctctctctctctctctctctctctccctctccctctccctctccctctccctctccctctctctctctctctctctctctctcccccccccttgggatttactttccaatccaaaagctttttaaaatttatttttatgcctttgtcaaaaaacaaaacaaacaaacaaacaaaaacccccatgtccctaaaagatacagtgtcagtaactgcaggcactgcacaggactgtaacttaaaactgtcaacagcctaatctgcagtaattgccttttaaaaaggagcctgcctctttaagccattcattctacgtgatttggtgagaatttcatctccaagcctgtagctgtagctctaaattgaccccataggtgtttggcctgaccccaggggggcgtggaaggtgtgggatttgtaccgtggagatcagaggaccagttgcccataaaggaaagtggaaacacaaaggttggctaaaaccaagaccggcagacagcagaacattgtttcctccctgccttccagtcagaagcaggtctccatccctccatccccgttagtataaagccatcagctcctactaaataccagaggtgagaaaatgacagcttttccccaacccccaactcacctcttctagctgctctgcaattcttccctgagtgatgtctaactagcagacaagggataaagccaagggtgagaaacggcagaagaggcagaaatcttgggagggaagggaaggaccgtctccactcctcagtcatctcaagctcagacagttcaacagagtttcagttttcttttgcttggggtcagcgggtctctggaacatcccacattgttgatttagtactaagaaagacaggcggaccattcgggtagttggaatccagcatttctttagcttctaaatatgattgtcccctagaactgaagttcaaggctttctggcataattatctgacttttggtcgcagacttcccagccctgtcgtgtctctagctgtcactggggtgttctgcgttttattttcaggtaccctctatgactttaatccagaagggaaaggcaagcctggaggtgggggtgtaatctgtgaccttcttctggttatgagggaaggggggagggagagattttactgttgaaaaaaaaatgtctctggtgcgggtggagacagacttctttcggctaattgtgtagtgaatggggagtaggaacaggggttgcatctcttatctcccccattactctctgcattgtcttctattgactaaggagcgagagatcttcttcctcctcatccctaccctctgctgtttctgcgaggtgtgctgagccagggctagatagggaatggagtgggctgatggtggcgggagtggccttgcatgctcaccgagtccctttgccccgtagccgaacccatctcggctaggcagccagctctgcggccgcacgtccacaagcagccggagaagaccacccgagtgcggactgtgctcaacgagaagcagctgcacaccttgcggacctgctatgccgccaaccctcggccagatgcgctcatgaaggagcaactagtggagatgacgggcctcagtcccagagtcatccgagtgtggtttcaaaacaagcggtgcaaggacaagaaacgcagcatcatgatgaagcagctccagcagcagcaacccaacgacaaaactgtgagtagccccgaagctaagcaaggaagggatgctaggagaccttggaagaccgggctagcggggcatccctgtgatctgatggattggtactctgtggagcaccagcaggagcccaaaccaaggtggacctctcacttcacctaaacccaggtgtatagtgtctactctctacaagaaggccgagatttggctggttagaaactgcagacgtctgagtggtgcccatcctcccccatccattgctgattccaaaaggggtcatggtcttcatcaccatgggcaccctggaacctaaaggacctagtggctccctccctcccagggcagatggtgcgaggtcttgaactcaggttctgcaagcattggcaccattagagttgctcccgttgtgaagggcaacatcttgagtgctgatctagaacatgattcaatatactttccatctatccagcattctggtctcaccccaactcttggcaagacttgccactgtttttgcatttgggagatcccttcaaggtgcccatgcagacactcccctcttccaagcttccttccagcctttggtatcattatgttaggcaacaacacactccccttccacccccctcccccacctcttgcttaagttaaggtaaaggttttttgttttttttttttaaactacttcagccctatttttaaatgccattttttaaaaaaaatccattgtcattaaacttggcaggcagaccgagactgggtaagggtactttctaagttagagcataatagcacagaagtagccagacccagaaaccttggggctagggtggggtaagtatgattctggaaggaagagtgcttaggggcagggggatgggttcagctaactcaaactgcctagaaaaccagtatcagaaggctgaagggagacagaccctggctggacctcagttttctagttctgggagactgtggcctcttaacgcaggtgggaggggttggggtagggcataagctgtgaaggtgaagcaaaaggtgatagatgcagcagaattgttttaataagatctcagccagtctcataatccactgcacagaagtagaaaaacaaaactgaactcaaactcatatccataggtacagcagattaactgagtcaatacagggccactgtgtagataagataaatacttgggtatatttacttagcacatgcagaccaggaagggagggagtttgctcattaacatgttgggatgggggggggcgttattcacagaatatccaggggatgacaggaactcccatggtggctgctagtccggagagacatgatggtggtttacaggctaacccagtagaggtgcaaagttaccagccgccctggaaagtactgagtgacttcgccttgcaaagcgacatagatcagcctgcttttcagcaactggtaagtgtctggctcccaaatgggaaaagactggatccctaacaaaaaaaggagactcgttttaactgttaggcattgaaaggtcccctggggtgagggagggtaccgggattagagagaaagcaggatgtggtggtgaagaggagggaggcagatggaaggaaaggagatctcttgtgaatctttgtttaaaactgagatgccacaatttaactatgtagcctaagtgtgtagtgtgatcttcctacaaaggaggcatatcttatgggttgagcttctcagtgcctaagtagttctgtgtacagaggtacagacagtggcaatgtcacatattttaggctgttctaaattacaaggacttcagagaccaccgtggcatttggatcagcaatcagtaagttctttgctttacctgtgtcttgtgactgattcaagtaaaaatgactgggatgcacgcccagtgtgatacccagttagaatgcagttgtgcataagcagcagggagctgtctgaacacaattctcttctatgtcaaccacgaggatagttatttaaatagtttggttgatatttattattgttatgtttctttcaaagcacagattgatctaaagcagaggattttaaaaatatttttaaacaaatgagttcagatatgacacaattacattcagtgtggcatgtatgccaaaaggaacatgagcattgtcagaggcaatgcatatttaacttgtgaaaacctgtcttagcactaaagggtctaacacattcatccatagcagagaattaattcattttacctataaaaggttaagataaagaaaaatttaaagatagatcggtgtggtgcatatgcttaaggttttttttcttcttccttaaaggatgatcagccccatagatactgttttattaaacacgaagctgagtcctaaaaaacacccctttctttagaagcacagttaatgcatcaattgagaataggtttccccccattctatcctgtgccctaagcatatttgctttctcttgtcaacagttataagggtgattggccagaggcaagtgcttaaaagggcttggtaccatgctctctctctctctctctctctctctctttctctctctctcctccccacccccgtgtgtgtgtgtgtgtgtgtgtgtgtgtgtgtgtgtgtgtgtgtgtgtgtgtgtatgtgtatgtgtaaatgggccaatctagtgagcaggcaaatttgagaagtcccctgacctcgcaaggagaaggtttaatagatagtagataagggattctggagccaaagagaaagtttttccttaaataaaacaaattgtccctaggaagcattctgtaggtaaactttgaataatagaaaaggaggtgagagggggcttggttcctcctgaaacttgagtgtcaggtgagaacggtgaagtggaacccgaggaaggggtaggagggtatggttatgggtgggggtgacagtttacgtcacactgaatttataatgagatgatgaatgtgtaggtctccttgtggaaaggggaggaggggtcaggcaatgagagcacactgtgaatccactaaagagttaagcattgttcctcaggagcctgccttgaatttctggcctagagaactttcctactccccgggctagtaggcagctggataattttatttcaagccttagagggagagttatgatgccactttacagtgggaaacacatttgctcttaaataatttaataggacacaatgttgggaattcagcttgggttaaaagagagctctttaggaagatggagaaatggagagcatttctttgcaagtgcttctcctgggcttcctttaaagcctgtttaaattctatgacaggcataggtatctttgttggtataccagttctacttacaggaacttataggcgtagactcaatgtaatacttttgtatagctcgggacactactgaagccccttttgctgacttctcaaccttccatacaggtcaatttttcagaaggaggaccaggctctaattctactggcagtgaagtagcatcgatgtcctcgcagctcccagatacacccaacagcatggtagccagtcctattgaggcatgaggaacattcattcagatgttttgttttgttttgttttgtttttttcccctgttggagaaagtgggaaatgacgttgaactccgaaataaaaagtatttaacgacccagtcaatggaaactgaatcaagaaatgaacgctccaggaagcgcatgaagtctgttctaatgacaaagtgatatggtagcaacactgtgaagacaatcatgggattttactagaataaaaacaaacaaacaaacaaaaccctaagcccaacatatgctattcaatgaccttaggagtacttaaaaaagaaaaagaaaaaaaaaagagagagagaccgtttttaaaacgtagaggatttatattcaaggatctcaaaaaatgcgcgttttcatttcactgcacatctagaggaagagcagaaacagagaatttcctagtccatcctattctgaatggtgctgtttctatattggtcactgccttgccaaacaggagctccggcacagagcggaagaaaccagcctcagtgacttgaaagtgtcctttcaggaaggcggagctgcgttggtttgcaatgtttttagttgactttgacaaggggttacgtgaaattctgggtctcttaagcatgccctgtagctggtttctcttttacgtttgcctctcctcccatccttttctttccttttctttatttctctttacaatttttttgagatccatcctctatcaagaagtctgaagcgactttaaaggtttttaaatttgtatttaaaaaccaacttataaagcattgcaacaaggttacctctattttgccacaagcgtctcgggattgtgtttgactcctgtctgtccaagaacttttcccccaaagatgtgtatagttattggttaaaatgactgttttcgctctttctggaaataaagaggaaaaaggaaactttttttgtttgctcttgcattgcaaaaattataaaagtaatttattatttattgtcaggagacttgccacttttcatgtcatttgactttttttttgtttgctgaagtaaaaagaagataaaggttgtaccgtggtctttgaattatatgtctaagtttatgtgttttgtcttttttttttctttaaatattatgtgaaatcaaagcgccatatgtagaattatatcttcaggactatttcactaataaacgtttggcatagataattaaataaacgcaatgattcagggtc

SEQ ID NO: 5 - DNA, 2526 bp, unidentified
  source 1..2526: mol_type = genomic DNA
  CDS 267..1316
tttgagtaagagataaggaagagaggtgccccgagccgtgcgagtccgccgctgctgctgcgcctccgctctgccaactccgccggcttaaatcggactcccagatctgcgagggcgcggcgcagccgggcagctgtttcccccagttttggcaaccccgggggccactatttgccacctagccacagcaccagcatcctctctgtgggctattcaccaattgtccaaccaccatttcactgtggacattactccctcttacagatatgggagacatgggcgatccaccaaaaaaaaaacgtctgatttccctgtgtgttggttgcggcaatcaaattcacgaccagtatattctgagggtttctccggatttggagtggcatgcagcatgtttgaaatgtgcggagtgtaatcagtatttggacgaaagctgtacgtgctttgttagggatgggaaaacctactgtaaaagagattatatcaggttgtacgggatcaaatgcgccaagtgcagcataggcttcagcaagaacgacttcgtgatgcgtgcccgctctaaggtgtaccacatcgagtgtttccgctgtgtagcctgcagccgacagctcatcccgggagacgaattcgccctgcgggaggatgggcttttctgccgtgcagaccacgatgtggtggagagagccagcctgggagctggagaccctctcagtcccttgcatccagcgcggcctctgcaaatggcagccgaacccatctcggctaggcagccagctctgcggccgcacgtccacaagcagccggagaagaccacccgagtgcggactgtgctcaacgagaagcagctgcacaccttgcggacctgctatgccgccaaccctcggccagatgcgctcatgaaggagcaactagtggagatgacgggcctcagtcccagagtcatccgagtgtggtttcaaaacaagcggtgcaaggacaagaaacgcagcatcatgatgaagcagctccagcagcagcaacccaacgacaaaactaatatccaggggatgacaggaactcccatggtggctgctagtccggagagacatgatggtggtttacaggctaacccagtagaggtgcaaagttaccagccgccctggaaagtactgagtgacttcgccttgcaaagcgacatagatcagcctgcttttcagcaactggtcaatttttcagaaggaggaccaggctctaattctactggcagtgaagtagcatcgatgtcctcgcagctcccagatacacccaacagcatggtagccagtcctattgaggcatgaggaacattcattcagatgttttgttttgttttgttttgtttttttcccctgttggagaaagtgggaaatgacgttgaactccgaaataaaaagtatttaacgacccagtcaatggaaactgaatcaagaaatgaacgctccaggaagcgcatgaagtctgttctaatgacaaagtgatatggtagcaacactgtgaagacaatcatgggattttactagaataaaaacaaacaaacaaacaaaaccctaagcccaacatatgctattcaatgaccttaggagtacttaaaaaagaaaaagaaaaaaaaaagagagagagaccgtttttaaaacgtagaggatttatattcaaggatctcaaaaaatgcgcgttttcatttcactgcacatctagaggaagagcagaaacagagaatttcctagtccatcctattctgaatggtgctgtttctatattggtcactgccttgccaaacaggagctccggcacagagcggaagaaaccagcctcagtgacttgaaagtgtcctttcaggaaggcggagctgcgttggtttgcaatgtttttagttgactttgacaaggggttacgtgaaattctgggtctcttaagcatgccctgtagctggtttctcttttacgtttgcctctcctcccatccttttctttccttttctttatttctctttacaatttttttgagatccatcctctatcaagaagtctgaagcgactttaaaggtttttaaatttgtatttaaaaaccaacttataaagcattgcaacaaggttacctctattttgccacaagcgtctcgggattgtgtttgactcctgtctgtccaagaacttttcccccaaagatgtgtatagttattggttaaaatgactgttttcgctctttctggaaataaagaggaaaaaggaaactttttttgtttgctcttgcattgcaaaaattataaaagtaatttattatttattgtcaggagacttgccacttttcatgtcatttgactttttttttgtttgctgaagtaaaaagaagataaaggttgtaccgtggtctttgaattatatgtctaagtttatgtgttttgtcttttttttttctttaaatattatgtgaaatcaaagcgccatatgtagaattatatcttcaggactatttcactaataaacgtttggcatagataattaaataaacgca

SEQ ID NO: 6 - protein, 349 aa, unidentified; identical to SEQ ID NO: 3

SEQ ID NO: 7 - DNA, 5753 bp, Homo sapiens
  source 1..5753: mol_type = genomic DNA
gctagtgcgtgttgacgtcatgctgcgtgcgggccggtgcggaatcgctccttcaactccgcggggcagtaggagttagttagcaaagagccgaggccgggcgcgcgaccctcgtccttctgcccctggccgcacactttgcgcacatctctttttctgcatggtggatattatttttcattatccttttctgggtgctatgggtgatcattccaagagtaagtatttctgtgtgtgtgtggggtggggtgtgtgtgtatgcttaatatgcaaaatttctaatgcagaaaaatgtttagtggattctacaagtggcttctatttgtcagaatagtttaggagaggaaaaacgaatgcatgtctcctgcaggactgcttatctttgggaatcctgttgttttattttataagtaaacactatttctcaagccggctaggatgacttcgggctgcgaaatctgcttctgttttgcgcaaaggcaattaggtttgatttagggatgtgggtttttttttttttggttgttgtttgtttgcctttcggtttttcaaagctaggaagctttctttcgcttgctctgggatatttaaactaagaaatgtctgagaggatgagcggggactgccgaaagacgatgtgtctgtgttgggataggaggcagccgctgtgagtgggagtgtgtgagcggaaatgccaggacagcaccgttaggtgttgatatccaggcgtccgcagtgcctgtgtatttactctcttcttagtttcgtgtttgcgtttagtttcaagacacgtcgacaaaacgactgtcattaatgaatgtaataacgaaatatttggggtgggggacagaagaaaccttcagcaatccacaagatactggtttttcgtttccaatgccctggacctttagagagctcatcatctttccttatggtcgcaagtcttgaaagaaagagcaagagagcgagcgggttgggttggggctggagtagccgaggccggcctgggtccgggcagtcaggcctgacgcggccccgcgcccttccccggcagagaagcccgggacggccatgtgcgtgggctgcgggagtcagatccacgaccagtttatcctgcgggtgtcgcccgacctcgagtggcacgcggcctgcctcaagtgtgccgagtgcagccagtacctggacgagacgtgcacgtgcttcgtgagagacgggaagacctactgcaagcgggactatgtcaggtgaggccggcgggaacgcgggctgggccaccgcgcgcaggggccggggccgggactggggatggcggcctgcccgcgcgccccggccctgcccagggagaaggccatccgcatcccgcacgggtgccgcagcgctcccccgggcccgggaatgcccgcaggcgggtcaccgcagtctcctggtggccacaaagtgtcctgggcgcgcgccggagccgtagcagccagggagatgagggcggccgcaggcccagcgctgacaccgccgcaccttgggcccgcaggctgttcggcatcaagtgcgccaagtgccaggtgggcttcagcagcagcgacctggtgatgagggcgcgggacagcgtgtaccacatcgagtgcttccgctgctccgtgtgcagccgccagctgctgcctggggacgagttctcgctgcgggagcacgagctgctctgccgcgccgaccacggcctcctgctcgagcgcgccgcggccggcagcccgcgcagccccggcccgcttcccggcgcccgcggcctgcatctgcccggtaagcgcgccggggcctgtgccggggtgagcggggtgtatgtgcgtgcgtgtgtgtaggggtacgcttgtgtccctaacgagaagttgtcagttgtgtgtggttccgtctgccgggatagtgtttttctgtatcagtgcggaactgtgtgctgggaacaaggctgtgtgtattctcgtgtgctcgggagaaactgcgtgtgtatgagtgtgtgagcacggagaattgtgcagaatcgtgttctccatgaccaggaacatgcagggcacagtgctaggagcagaagagtggatgacaacagggaatcttaagtggtgtccgtcttggggcttgtattaggaatttcttggaggaatatgtgttattgtcagagggtaaggtttgcccaggggtatgtgtgtaagtaacacagctccaaactgggagctgggcaggagcttggtcagccaggagccaagaacccaacaaagaggggtttttgccatcttgttttgaggcccagtttgatgaacaatttagccaggttcttctgggggagggggagtatcaatgcatagtggactgtcactggaatattctgggtcctaatcagcagctgcccagagatggggtctgagtgtataatgggcaggtacggctgaacagaaacagaggtgcctccaccccaggcaaggcagtttcccccaggtagccataggcctgcagcacagatccgtagaccaggctgggcctctgggtgctgtgtggccttgccccgctcctcccctctctggatcttcacttcctccctatagaaagaggaaactgggctggatgggctatttgtaaatatttgtccagacctagaaattcgtagaggggaacataaaccgaaatggtgcagtacagatccacggtatatatgggtcgggatcaaatggagatgtgaaatgggagcaagcgggtattcagtgcacccccatctgggatctcggagaggctcccctttgtttgtttcgggcttcagggtgcaggctctggaatcagtcactgtttccccgggatggagagaacctggggccaagcgaggagagtgccagcggctgctcagcaccttggtcggtgggaggccactgggctggggaacctggagccttgaggaaggagacaggggccgaagcccaagggcgggcaggaaggaactgttgcaagccctggaggcccgtccataaccgtggatgagtcttctacacatgtggcctggccctgtccaaatgggctctgaaggaggggaagggccctcgtaggcccccggcatattcgcacctcccacccagtcccctgggcagcgcctcgccctgggtccacgtcggtccctttccctctccagagtcctgggcaagcctctttcctcttcctctgaaactccgacttcttccggagggcttcgctcgttccgagggtcctgcgactggaggaggcacatccctgagcagccactccctccccgcagcggcctgtcgccgagctcccgcgccctgcttgagctcgggagagatcgctgcggggcagtccggcccgggagcgagagaaagaacgaaaatgcacagctctctccgagtaaggcgaacagatggagaggaagacgaacaattaaacgaaacaaacagaataaaacagaaacgaaatcgggccccggggggctgggccacccggaggttgggctcggggcgggtgggtggcggcccctcgctaacctctggcctcaccctgtcctggcagacgctgggtcgggccggcagcccgcgttgcgcccgcacgtgcacaagcagacggagaagacgacccgcgtgcggactgtgctgaacgagaagcagctgcacactctgcggacctgctacgccgccaacccgcggcccgacgctctcatgaaggagcagctggtggagatgaccggcctgagcccgcgggtcatccgcgtctggttccagaacaagcgctgcaaggacaagaagaaatccattctcatgaagcagctgcagcagcagcagcacagcgacaagacggtgagcagccgctgggccggaggctcgagtcgggtgggggctgcgcttccgccgcggtatctgcgtgcccttttctgggcgagccctgggagatccagggagaactgggcgctccagatggtgtatgtctgtaccttcacagcaaggcttcccttggatttgaggcttcctattttgtctgggatcggggtttctccttgtcccagtggcagccccgcgttgcgggttccgggcgctgcgcggagcccaaggctgcatggcagtgtgcagcgcccgccagtcgggctggtgggttgtgcactccgtcggcagctgcagaaaggtgggagtgcaggtcttgcctttcctcaccgggcggttggcttccagcaccgaggctgacctatcgtggcaagtttgcggcccccgcagatccccagtggagaaagagggctcttccgatgcgatcgagtgtgcgcctccccgcaaagcaatgcagaccctaaatcactcaaggcctggagctccagtctcaaaggtggcagaaaaggccagacctaactcgagcacctactgccttctgcttgccccgcagagccttcagggactgactgggacgcccctggtggcgggcagtcccatccgccatgagaacgccgtgcagggcagcgcagtggaggtgcagacgtaccagccgccgtggaaggcgctcagcgagtttgccctccagagcgacctggaccaacccgccttccaacagctggtgaggccctgccctacccgccccgacctcgggactctgcgggttggggatttagccacttagcctggcagagaggggagggggtggccttgggctgaggggctgggtacagccctaggcggtgggggagggggaacagtggcgggctctgaaacctcacctcggcccattacgcgccctaaaccaggtctccctggattaaagtgctcacaagagaggtcgcaggattaaccaacccgctcccccgccctaatccccccctcgtgcgcctggggacctggcctccttctccgcagggcttgctctcagctggcggccggtccccaagggacactttccgactcggagcacgcggccctggagcaccagctcgcgtgcctcttcacctgcctcttcccggtgtttccgccgccccaggtctccttctccgagtccggctccctaggcaactcctccggcagcgacgtgacctccctgtcctcgcagctcccggacacccccaacagtatggtgccgagtcccgtggagacgtgagggggacccctccctgccagcccgcggacctcgcatgctccctgcatgagactcacccatgctcaggccattccagttccgaaagctctctcgccttcgtaattattctattgttatttatgagagagtaccgagagacacggtctggacagcccaaggcgccaggatgcaacctgctttcaccagactgcagacccctgctccgaggactcttagtttttcaaaaccagaatctgggacttaccagggttagctctgccctctcctctcctctctacgtggccgccgctctgtctctccacgccccacctgtgtccccatctcggccggcccggagctcgcccacgcggacccccgccctgccccagctcagcgctccctggcggcttcgcccgggctcctagcggggaaaaggaaggggataactcagaggaacagacactcaaactcccaaagcgcatgattgctgggaaacagtagaaaccagacttgccttgaaagtgtttaagttattcgacggaggacagagtatgtgagcctttgccgaacaaacaaacgtaagttattgttatttattgtgagaacagccagttcatagtgggacttgtattttgatcttaataaaaaataataacccgggg

SEQ ID NO: 8 - DNA, 1918 bp, Homo sapiens
  source 1..1918: mol_type = genomic DNA
  CDS 161..1240
gctagtgcgtgttgacgtcatgctgcgtgcgggccggtgcggaatcgctccttcaactccgcggggcagtaggagttagttagcaaagagccgaggccgggcgcgcgaccctcgtccttctgcccctggccgcacactttgcgcacatctctttttctgcatggtggatattatttttcattatccttttctgggtgctatgggtgatcattccaagaagaagcccgggacggccatgtgcgtgggctgcgggagtcagatccacgaccagtttatcctgcgggtgtcgcccgacctcgagtggcacgcggcctgcctcaagtgtgccgagtgcagccagtacctggacgagacgtgcacgtgcttcgtgagagacgggaagacctactgcaagcgggactatgtcaggctgttcggcatcaagtgcgccaagtgccaggtgggcttcagcagcagcgacctggtgatgagggcgcgggacagcgtgtaccacatcgagtgcttccgctgctccgtgtgcagccgccagctgctgcctggggacgagttctcgctgcgggagcacgagctgctctgccgcgccgaccacggcctcctgctcgagcgcgccgcggccggcagcccgcgcagccccggcccgcttcccggcgcccgcggcctgcatctgcccgacgctgggtcgggccggcagcccgcgttgcgcccgcacgtgcacaagcagacggagaagacgacccgcgtgcggactgtgctgaacgagaagcagctgcacactctgcggacctgctacgccgccaacccgcggcccgacgctctcatgaaggagcagctggtggagatgaccggcctgagcccgcgggtcatccgcgtctggttccagaacaagcgctgcaaggacaagaagaaatccattctcatgaagcagctgcagcagcagcagcacagcgacaagacgagccttcagggactgactgggacgcccctggtggcgggcagtcccatccgccatgagaacgccgtgcagggcagcgcagtggaggtgcagacgtaccagccgccgtggaaggcgctcagcgagtttgccctccagagcgacctggaccaacccgccttccaacagctggtctccttctccgagtccggctccctaggcaactcctccggcagcgacgtgacctccctgtcctcgcagctcccggacacccccaacagtatggtgccgagtcccgtggagacgtgagggggacccctccctgccagcccgcggacctcgcatgctccctgcatgagactcacccatgctcaggccattccagttccgaaagctctctcgccttcgtaattattctattgttatttatgagagagtaccgagagacacggtctggacagcccaaggcgccaggatgcaacctgctttcaccagactgcagacccctgctccgaggactcttagtttttcaaaaccagaatctgggacttaccagggttagctctgccctctcctctcctctctacgtggccgccgctctgtctctccacgccccacctgtgtccccatctcggccggcccggagctcgcccacgcggacccccgccctgccccagctcagcgctccctggcggcttcgcccgggctcctagcggggaaaaggaaggggataactcagaggaacagacactcaaactcccaaagcgcatgattgctgggaaacagtagaaaccagacttgccttgaaagtgtttaagttattcgacggaggacagagtatgtgagcctttgccgaacaaacaaacgtaagttattgttatttattgtgagaacagccagttcatagtgggacttgtattttgatcttaataaaaaataataacccgggaaaaaaaaaaaaaaaaaaaaaaaaaaaaaaaaaaaaaa

SEQ ID NO: 9 - protein, 359 aa, Homo sapiens
MVDIIFHYPFLGAMGDHSKKKPGTAMCVGCGSQIHDQFILRVSPDLEWHAACLKCAECSQYLDETCTCFVRDGKTYCKRDYVRLFGIKCAKCQVGFSSSDLVMRARDSVYHIECFRCSVCSRQLLPGDEFSLREHELLCRADHGLLLERAAAGSPRSPGPLPGARGLHLPDAGSGRQPALRPHVHKQTEKTTRVRTVLNEKQLHTLRTCYAANPRPDALMKEQLVEMTGLSPRVIRVWFQNKRCKDKKKSILMKQLQQQQHSDKTSLQGLTGTPLVAGSPIRHENAVQGSAVEVQTYQPPWKALSEFALQSDLDQPAFQQLVSFSESGSLGNSSGSDVTSLSSQLPDTPNSMVPSPVET

SEQ ID NO: 10 - DNA, 1057 bp, Homo sapiens
  source 1..1057: mol_type = genomic DNA
tgattggacagcagcgaggtcccaccgggcgcctccgcggcttaaaaccgaagcgggggcgagggggaggtagagcccgccccctgcgcgtcgcggctgggagggagaggaggtgggagagaaggaagaggcggaggaggcagcgggcgccgaggctctgggacccgcaggcaagccagaccgacgcgcagagccgggagcctcatcgcagcggcagcggcagcggcgggctcgggccggcctcgggggctgctcacccacatgagcatccgcccacccggcgagcccccgagcccaggcggcgcggccatggccgagctcaagtcgctgtcgggggacgcgtacctggcactaagccacggctacgcggcggcggctgcgggtctcgcctacggggcggcgcgagaacccgaagcggcccgcggctacggcactccgggcccgggcggcgacctccccgcggcgcctgcacctcgcgccccagctcaggcggcggagagcagcggcgaacagagcggggacgaggacgacgccttcgagcagcggcggcggcggcgcgggccagggagcgcggcggacgggcggcggcggccgcgagagcagcggtctctgcggctcagcatcaacgcgcgcgagcggcggcgcatgcacgacctaaacgacgcgctggacgggctgcgagccgtcatcccctacgcgcacagcccgtcggtgcgcaagctctccaagatcgccacgctgctgctcgccaagaactatatcctcatgcaggcgcaggccctggacgagatgcggcgcctggtggccttcctcaaccagggccagggcctggccgcgcccgtaaacgccgcgcccttgacgcccttcggccaggccactgtgtgccccttctccgcaggcgccgccctggggccctgccctgacaagtgcgccgccttctccgggacgccctccgcgctttgcaaacactgtcacgagaagccgtgaccgcgcgggcccccggccctcccgcacgcgtcctccgatcgcccctgtgactgtctctctgcccggacag

SEQ ID NO: 11 - DNA, 1057 bp, Homo sapiens; identical to SEQ ID NO: 10
  source 1..1057: mol_type = genomic DNA
  CDS 262..987

SEQ ID NO: 12 - protein, 241 aa, Homo sapiens
MSIRPPGEPPSPGGAAMAELKSLSGDAYLALSHGYAAAAAGLAYGAAREPEAARGYGTPGPGGDLPAAPAPRAPAQAAESSGEQSGDEDDAFEQRRRRRGPGSAADGRRRPREQRSLRLSINARERRRMHDLNDALDGLRAVIPYAHSPSVRKLSKIATLLLAKNYILMQAQALDEMRRLVAFLNQGQGLAAPVNAAPLTPFGQATVCPFSAGAALGPCPDKCAAFSGTPSALCKHCHEKP

SEQ ID NO: 13 - DNA, 2520 bp, unidentified
  source 1..2520: mol_type = genomic DNA
agagcggccccggccggccacgcgccttggtgagaaggcgcaggaagggtggccggccggagaggccacgcgcaggcccggggactgacagacagctggacaggtggacgcgcccgcagggcaagaagccatactgccgtagcgggctccagaggtcatggccgagctcaagtcgctgtcgggggactcgtacctggcgctgagccacagctacacggcgaccggccacgcctacgctgcagcgcgcggaccggagaccacacgcggcttcggtgcatcgggtccgggtggcgacctcccggcggctcccgcttctcgtgtccccgccgcgacggtggagagcagcggcgagcagagcggcgacgaggacgaagccttcgagaggcggcggcggcggcgagggtccggggttgcggttgacgcacggcggcggccccgggagcagcgctcgctgaggctcagcatcaacgcgcgcgaacggcgacgcatgcatgacctgaacgacgcgctggacggactgcgcgcagtcatcccctacgcgcacagcccgtcggtgcgcaagctctccaagatagccacacttctgctggccaaaaactacatcctcatgcaggcgcaggccctggaggagatgcggcgcttggtagcatacctcaaccaaggccagggcctcgcggcgccggtggccgccgcgcctctaacgccctttggccaggcagcgatttacccgttctcggcgggcaccgcgctcgggccctgcccagacaagtgtgccaccttctcaggatcgccctcggcgctttgcaaacactgcggcgagaagccctgagggctccgatcccctctgcagccgcctttctatttagactgttccctgtcctcgctggcgggactctatgagaaagcccagtactccgcttacagaactgttgggtgcagaatgagaagtccgtctgagaaagccagagagttggcgctggactctagaccctgccgcatccactgaactatctcagctaaaaggctacgggagtgagcagttagtgaatggtcccacatctgacaggtgactgcccaaggctcaggagcgcaggcacagagcatcgcgcttgaccacatcctagcgttggatggggaacccagccccttctaagccaacttgcagggccttgcttgcttcaactagacctgttcctcaggacgggtgcggcccctcacctcgggcctctgttggctccaagactccagacccggcctaaaagcaaaattacgccgaggtcctttggagaggtcgtggaggcagacgcaggaagatcccaggaggtgagctttacaacgttttccaagtctgagcttgttctgttaacattcttgcctgcccagaggctcaggaaattcaagccagcctcaaaagtctgtggaaacatctctaaataagcctggagcgccaccgccggcctcctgtgggctgagctgggtttcacttctttctacttgtcccaaggggactggtggagccctcacaacctgctcagggctacttgaccctggagggtcagccgaagagtctagtctagctccaggaccaaccaagtcccctctgcgccagttcgggaacttctggaggaagccgggctaccggtgagtacccggtgaggtgttggactgaatgggaactggtcgctccccaaatcggaccacagcccagaatcctgccactgtacctagtctgagccttagcccgcgcgtctcagatgttgcctggttttcctctgtggccatcagcagcttccgcctccatttacccagttttcttggctttcattgatgcaaagcaaatcgggagagggccactgctcagcaaagcgcctgtctatagcgcttaacttgaattaacgcattggaaaaggcccttttgtatttgcaaaatgacgtgtgtgattaagtcttattcataaagacgtatgcttaatactggtttctgctaatttctattttttatttcttaagtgtctttcccctcccctaaaataatctattttgcacatttcaaaacagactgtttttcaagttcgcttaagatgaacagggaaaactggaaactcaagtgttgttagctctggggtcattacccactattatttttcaaagtcttttatttatatttagacgaggggtagtgtgcagaataaagtacttaaaacttatttttttaaaaatatgattaaaatttaatatcaaattaagtgtacatctatgatttgattcataatttcaattgtcagacagtgatactggtttggaatccttctacggccccaagaaatgctgagattttacaatggatccatactccattaagagatgtgtgagttcatacgaaaaatgtgtaggagtgttgggtggagacacaggggtgatcccaacctgcaggaggcagagacaggggcatggggattctgagaccagcctgagcaacacagggaggctctcttaggacaagaaaacaattaactttga

SEQ ID NO: 14 - DNA, 2520 bp, unidentified; identical to SEQ ID NO: 13
  source 1..2520: mol_type = genomic DNA
  CDS 158..829

SEQ ID NO: 15 - protein, 223 aa, unidentified
MAELKSLSGDSYLALSHSYTATGHAYAAARGPETTRGFGASGPGGDLPAAPASRVPAATVESSGEQSGDEDEAFERRRRRRGSGVAVDARRRPREQRSLRLSINARERRRMHDLNDALDGLRAVIPYAHSPSVRKLSKIATLLLAKNYILMQAQALEEMRRLVAYLNQGQGLAAPVAAAPLTPFGQAAIYPFSAGTALGPCPDKCATFSGSPSALCKHCGEKP

SEQ ID NO: 16 - DNA, 20 bp, synthetic construct
  misc_feature 1..20: note = gRNA for Bhlhb4
  source 1..20: mol_type = other DNA
ccgagctcaagtcgctgtcg

SEQ ID NO: 17 - DNA, 20 bp, synthetic construct
  misc_feature 1..20: note = gRNA for Bhlhb4
  source 1..20: mol_type = other DNA
cgcgccttggtgagaaggcg

SEQ ID NO: 18 - DNA, 20 bp, synthetic construct
  misc_feature 1..20: note = gRNA for mouse ISL1
  source 1..20: mol_type = other DNA
tcttcaatagcacgcgggaa

SEQ ID NO: 19 - DNA, 20 bp, synthetic construct
  misc_feature 1..20: note = gRNA for mouse ISL1
  source 1..20: mol_type = other DNA
tcctaagccataaagcgctt

SEQ ID NO: 20 - DNA, 20 bp, synthetic construct
  misc_feature 1..20: note = gRNA for Human ISL1
  source 1..20: mol_type = other DNA
ccaactccgccggcttaaat

SEQ ID NO: 21 - DNA, 20 bp, synthetic construct
  misc_feature 1..20: note = gRNA for Human ISL1
  source 1..20: mol_type = other DNA
gggaggttaatacttcggag

SEQ ID NO: 22 - DNA, 27 bp, unidentified
  source 1..27: mol_type = other DNA
tccgccggcttaaattggactcctaga

SEQ ID NO: 23 - DNA, 28 bp, unidentified
  source 1..28: mol_type = other DNA
tccgccggcttaggagagggagtgaaag

SEQ ID NO: 24 - DNA, 27 bp, unidentified
  source 1..27: mol_type = other DNA
tccgccggcttagagagggagtgaaag